(19) United States
(12) Patent Application Publication
(10) Pub. No.: US 2025/0250579 A1
(43) Pub. Date: Aug. 7, 2025

(54) REGULATORY SEQUENCES FOR MODULATING TRANSGENE EXPRESSION IN PLANTS

(71) Applicants: CORTEVA AGRISCIENCE LLC, INDIANAPOLIS, IN (US); PIONEER HI-BRED INTERNATIONAL, INC., JOHNSTON, IA (US)

(72) Inventors: SCOTT DIEHN, WEST DES MOINES, IA (US); AJIT NOTT, JOHNSTON, IA (US); DAVID SELINGER, HOCKESSIN, DE (US); CARL SIMMONS, DES MOINES, IA (US); PRIYANKA BHYRI, JOHNSTON, IA (US); VENKATA S TAVVA, HYDERABAD (IN)

(73) Assignees: CORTEVA AGRISCIENCE LLC, INDIANAPOLIS, IN (US); PIONEER HI-BRED INTERNATIONAL, INC., JOHNSTON, IA (US)

(21) Appl. No.: 19/092,579

(22) Filed: Mar. 27, 2025

Related U.S. Application Data
(63) Continuation of application No. 17/645,631, filed on Dec. 22, 2021, now Pat. No. 12,286,634.
Application No. 17/645,631 is a continuation of application No. 16/429,846, filed on Jun. 3, 2019, now Pat. No. 11,242,535.

(51) Int. Cl. C12N 15/82

(52) U.S. Cl. CPC C12N 15/8216

(57) ABSTRACT

The invention relates to gene expression regulatory sequences, specifically introns that act as enhancers of gene expression, the promoter and terminator sequences endogenously associated with these introns. Presence of these intronic enhancer sequences in proximity to promoter sequences leads to enhancement of gene expression. Methods of finding such new intronic enhancer sequences and using them to generate transgenic plants are also described.

### CROSS REFERENCE TO RELATED APPLICATIONS

This application claims the benefit of Indian Provisional Application No. 1340/DELNP/2010, filed Jun. 9, 2010, and U.S. Provisional Application No. 61/372,515, filed Aug. 11, 2010, the entire contents of each is herein incorporated by reference.

### FIELD OF INVENTION

The present invention relates to the generation of transgenic plants, particularly to the use of promoter and intron sequences to regulate gene expression in plants.

### BACKGROUND

Recent advances in plant genetic engineering have opened new doors to engineer plants to have improved characteristics or traits. These transgenic plants characteristically have recombinant DNA constructs in their genome that have a protein-coding region operably linked to at least one regulatory region that is the promoter. The promoter can be a strong or weak promoter, or a constitutive or tissue-specific promoter. Besides the promoter, the expression level of the gene product can be modulated by other regulatory elements such as introns. Introns are intervening, non-coding sequences that are present in most eukaryotic genes. Introns have been reported to affect the levels of gene expression. This effect is known as Intron Mediated Enhancement (IME) of gene expression (Lu et al., Mol Genet Genomics (2008) 279:563-572). Callis et al. (Genes Dev. 1987 1:1183-1200) showed that the presence of the first intron from maize alcohol dehydrogenase-1 (Adh1) gene increased the expression levels of transgenes in cultured maize cells up to 100-fold when compared to intronless constructs. Mascarenkas et al. (Plant Mol. Biol., 1990, 15:913-920) showed that other introns from the maize Adh1 gene could also increase heterologous gene expression in maize protoplasts. Vasil et al. (Plant Physiol., 1989, 91:1575-15790) reported that the constructs containing Shrunken-1 (Sh-1) first intron had much higher expression levels of the reporter gene in plant protoplasts, when compared to the constructs with promoter alone, or to constructs with promoter and Adh-1 first intron. Identifying novel regulatory sequences can lead to finer modulation of gene expression in transgenic plants.

Plant genetic engineering has advanced to introducing multiple traits into commercially important plants, also known as gene stacking. This is accomplished by multigene transformation, where multiple genes are transferred to create a transgenic plant that might express a complex phenotype, or multiple phenotypes. But it is important to modulate or control the expression of each transgene optimally. The regulatory elements such as the promoter and the terminator sequences need to be diverse, to avoid introducing into the same transgenic plant repetitive sequences, which has been correlated with undesirable negative effects on transgene expression and stability (Peremarti et al (2010) Plant Mol Biol 73:363-378; Mette et al (1999) EMBO J 18:241-248; Mette et al (2000) EMBO J 19:5194-5201; Mourrain et al (2007) Planta 225:365-379, U.S. Pat. Nos. 7,632,982, 7,491,813, 7,674,950, PCT Application No. PCT/US2009/046968). Therefore it is important to discover and characterize novel regulatory elements that can be used to express heterologous nucleic acids in important crop species. Diverse regulatory regions can be used to control the expression of each transgene optimally.

### SUMMARY

The present invention relates to regulatory sequences for modulating gene expression in plants. Recombinant DNA constructs comprising regulatory sequences are provided. Recombinant DNA constructs comprising intron sequences acting as enhancers of gene expression and endogenous promoter and terminator sequences corresponding to these intron sequences are provided.

Another embodiment of the invention is a recombinant DNA construct comprising an intron operably linked to a promoter and a terminator wherein the intron comprises a nucleotide sequence that has at least 95% sequence identity to SEQ ID NO: 4, 8, 13, 19, 52, 53, 56, 57, 58, 101, 102, 103, 104, 118, 137 or 138. In another embodiment, the intron comprises the nucleotide sequence of SEQ ID NO: 4, 8, 13, 19, 52, 53, 56, 57, 58, 101, 102, 103, 104, 118, 137 or 138.

One embodiment of the invention is a recombinant DNA construct comprising an intron operably linked to a promoter and a terminator wherein the promoter comprises a nucleotide sequence that has at least 95% sequence identity to SEQ ID NO: 105-117, 119, 136 or 139. In another embodiment, the promoter comprises the nucleotide sequence of SEQ ID NO: 105-117, 119, 136 or 139.

One embodiment of the invention is a recombinant DNA construct comprising an intron operably linked to a promoter and a terminator wherein the terminator comprises a nucleotide sequence that has at least 95% sequence identity to SEQ ID NOS: 140, 141, 142 or 143. In another embodiment, the terminator comprises the nucleotide sequence of SEQ ID NO: 140, 141, 142 or 143.

One embodiment of the invention is a recombinant DNA construct comprising an intron operably linked to a promoter and a terminator wherein the intron comprises a nucleotide sequence that has at least 95% identity to SEQ ID NOS: 4, 8, 13, 19, 52, 53, 56, 57, 58, 101, 102, 103, 104, 118, 137 or 138; and the promoter comprises a nucleotide sequence that has at least 95% identity to SEQ ID NOS: 105, 106, 107, 108, 109, 110, 111, 112, 113, 114, 115, 116, 117, 119, 136 or 139.

One embodiment of the invention is a recombinant DNA construct comprising an intron operably linked to a promoter and a terminator wherein the intron comprises a nucleotide sequence that has at least 95% identity to SEQ ID NO: 4, 8, 13, 19, 52, 53, 56, 57, 58, 101, 102, 103, 104, 118, 137 or 138; the promoter sequence has at least 95% identity to SEQ ID NO: 105, 106, 107, 108, 109, 110, 111, 112, 113, 114, 115, 116, 117, 119, 136 or 139; and the terminator has at least 95% sequence identity to SEQ ID NO: 140, 141, 142 or 143.

In one embodiment of the current invention, the intron is operably linked to the promoter, and is present downstream of the promoter, in the recombinant DNA constructs described herein. One embodiment of the present invention includes a recombinant DNA construct comprising an intron described in the present invention, operably linked to a promoter and a heterologous polynucleotide, wherein the intron can act as enhancer of expression of the heterologous polynucleotide.

Another embodiment of the invention encompasses a recombinant DNA construct comprising an intron wherein the intron sequence comprises at least one copy of the 8-bp sequence motif of SEQ ID NO: 99; or contains at least one copy of the 8-bp sequence motif of SEQ ID NO: 99 and at least one copy of the 5-bp sequence motif of SEQ ID NO: 100, wherein the intron is capable of enhancing expression of a heterologous polynucleotide in a transgenic plant. The intron sequence can also comprise more than one copy of SEQ ID NO: 99, or can comprise one or more than one copy of SEQ ID NO: 99 and more than one copy of SEQ ID NO: 100.

Another embodiment of this invention is a method to identify novel introns that are useful for enhancing expression of a heterologous polynucleotide in a plant cell, the method comprising the steps of scanning a plurality of introns from plants for presence of SEQ ID NO: 99, selecting a sequence that contains at least one copy of SEQ ID NO: 99, measuring the efficacy of the identified intron to enhance expression of a heterologous polynucleotide in a plant.

Another embodiment of the invention is a method for identifying novel intronic sequences for enhancing transgene expression in monocotyledenous plants by identifying sequences orthologous to SEQ ID NO: 4, 8, 13, 19, 52, 53, 56, 57, 58, 101, 102, 103, 104, 118, 137 or 138; and measuring the enhancing effect of the identified intron on the expression of an operably linked heterologous polynucleotide.

Another embodiment of the current invention includes the promoter and the terminator sequences that are endogenously linked to the introns identified using the methods described in the current invention.

Another embodiment of the current invention is a method for modulating expression of a heterologous polynucleotide in a monocotyledonous plant comprising the steps of: (a) introducing into a regenerable plant cell a recombinant DNA construct comprising a promoter and a heterologous polynucleotide wherein each is operably linked to an intron, wherein the intron comprises either (i) a nucleotide sequence that is orthologous to SEQ ID NO: 4, 8, 13, 19, 52, 53, 56, 57, 58, 101, 102, 103, 104, 118, 137 or 138; or (ii) a nucleotide sequence that contains least one copy of a sequence motif identical to SEQ ID NO: 99; and (b) regenerating a transgenic plant from a regenerable monocotyledonous plant cell after step (a) wherein the transgenic plant comprises the recombinant DNA construct; and (c) obtaining a progeny plant derived from the transgenic plant of step (b), wherein said progeny plant comprises the recombinant DNA construct and exhibits enhanced expression of the heterologous polynucleotide when compared to a plant comprising a corresponding recombinant DNA construct without the intron sequence.

In another embodiment, this invention concerns a vector, cell, plant, or seed comprising a recombinant DNA construct comprising the regulatory sequences described in the present invention.

The invention encompasses regenerated, mature and fertile transgenic plants comprising the recombinant DNA constructs described above, transgenic seeds produced therefrom, T1 and subsequent generations. The transgenic plant cells, tissues, plants, and seeds may comprise at least one recombinant DNA construct of interest.

In one embodiment, the plant comprising the regulatory sequences described in the present invention is a monocotyledenous plant. In another embodiment, the plant comprising the regulatory sequences described in the present invention is a maize plant.

### BRIEF DESCRIPTION OF DRAWINGS AND SEQUENCE LISTINGS

The invention can be more fully understood from the following detailed description and the accompanying drawings and Sequence Listing which form a part of this application. The Sequence Listing contains the one letter code for nucleotide sequence characters and the three letter codes for amino acids as defined in conformity with the IUPAC-IUBMB standards described in Nucleic Acids Research 13:3021-3030 (1985) and in the Biochemical Journal 219 (No. 2): 345-373 (1984), which are herein incorporated by reference in their entirety. The symbols and format used for nucleotide and amino acid sequence data comply with the rules set forth in 37 C.F.R. § 1.822.

FIG. 1 is a schematic representation of the vector used for testing introns showing the location of restriction sites used to clone introns relative to the maize ubiquitin promoter, as described in Example 2.

FIG. 2 shows the map of PHP 41353, the ITVUR-2 vector used for testing intron-mediated enhancement of gene expression.

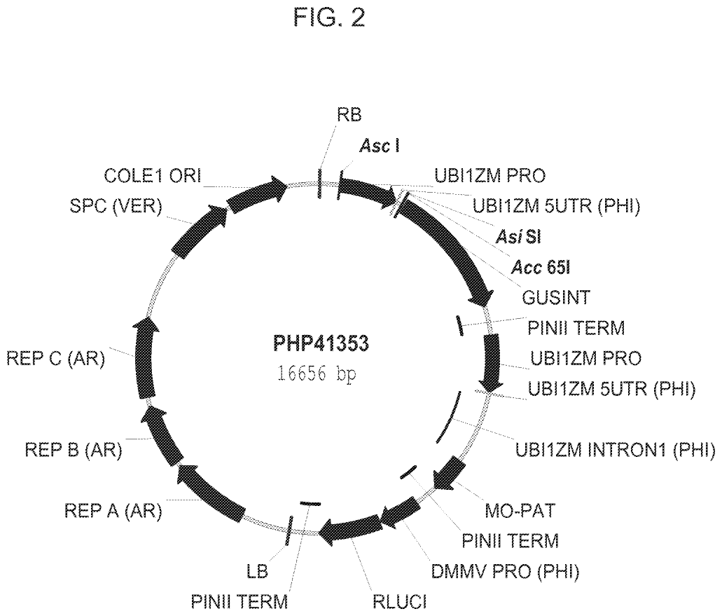

FIG. 3 shows quantitative analysis of GUS reporter gene expression in Maize Embryos infected with the respective constructs.

FIG. 4 shows the fold enhancement of GUS reporter gene expression in rice calli infected with intron constructs when compared with the control vector ITVUR-2.

FIG. 5 shows the map of PHP38808, the vector with CYMV promoter and ADH1 intron, used for testing intron-mediated enhancement of gene expression, as described in Example 7.

FIG. 6 shows the results of Northern blot of RNA extracted from infiltrated maize tissue culture material and probed with a digoxigenin-labeled DNA probe for the insecticidal gene used. Samples were loaded based on ELISA data to contain equal amounts of PAT.

FIG. 7 shows the map of PHP34651, vector containing GATEWAY® attR recombination sites and a PAT expression cassette used for LR reactions to generate the final expression vectors for introns, as described in Example 7.

FIG. 8 shows the map of PHP42365, vector containing ZmUbi promoter and ZmUbi intron, for testing in stable transgenic rice plants, as described in Example 11.

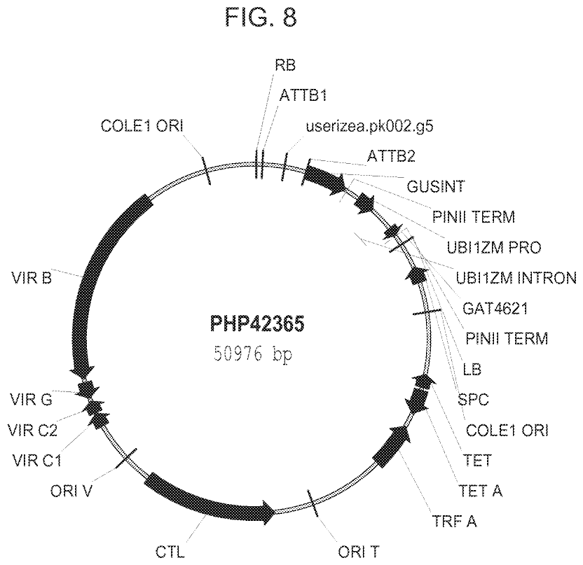

FIG. 9 shows MUG data from stable transgenic lines transformed with different constructs. Data represents the average of 5-8 independent single copy events+SE.

FIG. 10 shows MUG data from stable transgenic lines transformed with different constructs. Data represents the average of 5-8 independent single copy events+SE.

FIG. 11 shows histochemical data from leaves and panicles collected from stable transgenic lines transformed with different constructs. Representative images are shown for each construct analyzed.

FIG. 12 shows histochemical data from leaves and panicles collected from stable transgenic lines transformed with different constructs. Representative images are shown for each construct analyzed.

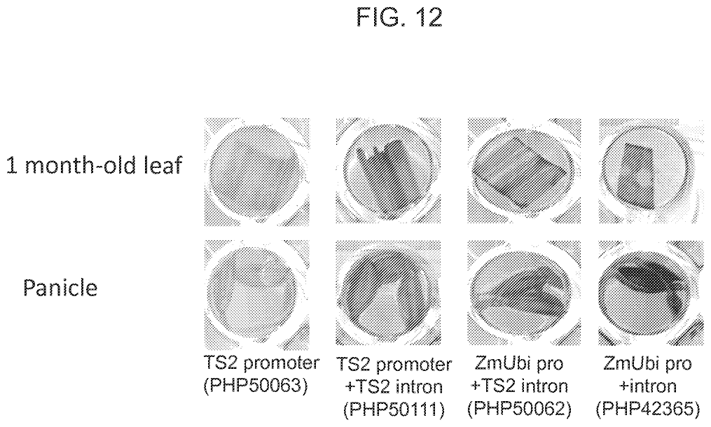

FIG. 13 is the schematic representation of the PHP49597 vector (terminator test vector).

SEQ ID NO: 1 is the sequence of the maize ubiquitin promoter.

SEQ ID NO: 2 is the sequence of the first intron from maize ubiquitin gene.

SEQ ID NO: 3 is the nucleotide sequence of PHP41353, ITVUR-2 vector.

SEQ ID NOS: 4-19 and SEQ ID NOS: 52-58, SEQ ID NO: 118, SEQ ID NOS: 137 and 138 are sequences of introns that were tested to identify expression-enhancing introns, and are described in Table 1 below.

SEQ ID NOS: 105-113, SEQ ID NO: 119 and SEQ ID NOS: 136 and 139 are the sequences of promoters identified for the enhancing introns as described in Example 10 and Example 11, and are described in Table 1 below.

SEQ ID NOS: 140-143 given in Table 1 are the sequences of the endogenous terminators for the introns TS1, TS2, TS13 and TS27, identified as explained in Example 13.

TABLE 1

| SEQ ID | | Intron/ | Enhancing/Non-Enhancing |
|---|---|---|---|
| NO | Name | Promoter | Intron |
| 4 | TS1 | Intron | Enhancing |
| 5 | TS4 | Intron | Non-Enhancing |
| 6 | TS5 | Intron | Non-Enhancing |
| 7 | TS6 | Intron | Non-Enhancing |
| 8 | TS7 | Intron | Enhancing* |
| 9 | TS8 | Intron | Non-Enhancing |
| 10 | TS10 | Intron | Non-Enhancing |
| 11 | TS11 | Intron | Non-Enhancing |
| 12 | TS12 | Intron | Non-Enhancing |
| 13 | TS13 | Intron | Enhancing |
| 14 | TS14 | Intron | Non-Enhancing |
| 15 | TS15 | Intron | Non-Enhancing |
| 16 | TS16 | Intron | Non-Enhancing |
| 17 | TS17 | Intron | Non-Enhancing |
| 18 | TS24 | Intron | Non-Enhancing |
| 19 | TS27 | Intron | Enhancing* |
| 52 | i1 | Intron | Enhancing |
| 53 | i2 | Intron | Enhancing |
| 54 | i3 | Intron | Non-Enhancing |
| 55 | i4 | Intron | Non-Enhancing |
| 56 | i5 | Intron | Enhancing |
| 57 | i6 | Intron | Enhancing |
| 58 | i7 | Intron | Enhancing |
| 105 | pTS1 | Promoter | Promoter identified for |
| | | | SEQ ID NO: 4 |
| 106 | pTS7 | Promoter | Promoter identified for |
| | | | SEQ ID NO: 8 |
| 107 | pTS13 | Promoter | Promoter identified for |
| | | | SEQ ID NO: 13 |
| 108 | pTS27 | Promoter | Promoter identified for |
| | | | SEQ ID NO: 19 |
| 109 | pi1 | Promoter | Promoter identified for |
| | | | SEQ ID NO: 52 |
| 110 | pi2 | Promoter | Promoter identified for |
| | | | SEQ ID NO: 53 |
| 111 | pi5 | Promoter | Promoter identified for |
| | | | SEQ ID NO: 56 |
| 112 | pi6 | Promoter | Promoter identified for |
| | | | SEQ ID NO: 57 |
| 113 | pi7 | Promoter | Promoter identified for |
| | | | SEQ ID NO: 58 |
| 118 | TS2 | Intron | Enhancing |
| 119 | pTS2 | Promoter | Promoter identified for |
| | | | SEQ ID NO: 118 |
| 136 | pTS1v | Promoter | Promoter sequence cloned |
| | | | for SEQ ID NO: 4 |
| 137 | TS7v | Intron | Enhancing |
| 138 | TS27v | Intron | Enhancing |
| 139 | pTS27v | Promoter | Promoter sequence cloned |
| | | | for SEQ ID NO: 19 |
| 140 | tTS1 | Terminator | Terminator identified for |
| | | | SEQ ID NO: 4 |
| 141 | tTS2 | Terminator | Terminator identified for |
| | | | SEQ ID NO: 118 |
| 142 | tTS13 | Terminator | Terminator identified for |
| | | | SEQ ID NO: 13 |
| 143 | tTS27 | Terminator | Terminator identified for |
| | | | SEQ ID NO: 19 |

*based on results from variants

SEQ ID NOS: 20-51 are the primers used for cloning introns as described in Table 2 in Example 3.

SEQ ID NO: 59 is the sequence of the vector PHP38808, used for testing intron-mediated enhancement of gene expression as described in Example 7.

SEQ ID NO: 60 is the sequence of PHP34651, the vector containing GATEWAY® attR recombination sites and a PAT expression cassette used for LR reactions to generate the final expression vectors for introns, as described in Example 7.

SEQ ID NOS: 61-94 are the oligonucleotides used for generating introns by oligonucleotide stacking as described in Table 4 in Example 7.

SEQ ID NO: 95 is the sequence for first intron of adh1 gene.

SEQ ID NO: 96 is the sequence for intron 6 for adh1 gene.

SEQ ID NO: 97 is the sequence for intron 1 for shrunken1 (Sh-1) gene

SEQ ID NO: 98 is the sequence for ubi intron 1 used for computational analyses as described in Example 8.

SEQ ID NO: 99 is the sequence of the 8-bp motif identified as described in Example 8.

SEQ ID NO: 100 is the sequence of the 5-bp motif identified as described in Example 8.

SEQ ID NOS: 101-104 are the intron sequences containing the 8-bp motif (SEQ ID NO: 99), as described in Example 9.

SEQ ID NOS: 114-117 are the promoter sequences identified from the introns of SEQ ID NOS: 101-104 respectively, as described in Examples 9 and 10.

SEQ ID NOS: 120-128 are the sequences of the primers used for cloning the promoters and introns, as described in Table 7.

SEQ ID NOS: 129-134 are the primer and probe sequences for qPCR, as described in Table 9 and Table 10.

SEQ ID NO: 135 is the sequence of the PHP42365 vector that contains ZmUbi promoter and ZmUbi intron.

SEQ ID NO: 144 is the sequence of the PHP49597 vector (terminator test vector or TTV).

SEQ ID NO: 145 corresponds to the nucleotide sequence

GATCAAAAAAAAAAAAA of a ‘promiscuous’ MPSS tags.

SEQ ID NO: 146 corresponds to the nucleotide sequence of a consensus motif sequence, which encompasses variations of the motif sequence given in SEQ ID NO: 99.

The sequence descriptions and Sequence Listing attached hereto comply with the rules governing nucleotide and/or amino acid sequence disclosures in patent applications as set forth in 37 C.F.R. § 1.821-1.825. The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (2): 345-373 (1984) which are herein incorporated by reference. The symbols and format used for nucleotide and amino acid sequence data comply with the rules set forth in 37 C.F.R. § 1.822.

### DETAILED DESCRIPTION OF THE INVENTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

The terms “monocot” and “monocotyledonous plant” are used interchangeably herein. A monocot of the current invention includes the Gramineae.

The terms “dicot” and “dicotyledonous plant” are used interchangeably herein. A dicot of the current invention includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably to refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from an mRNA template using the enzyme reverse transcriptase. The cDNA can be single-stranded or converted into the double-stranded form using the Klenow fragment of DNA polymerase I.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

“Mature” protein refers to a post-translationally processed polypeptide; i.e., one from which any pre- or pro-peptides present in the primary translation product has been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques.

“Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

The terms “entry clone” and “entry vector” are used interchangeably herein.

The term “insecticidal gene” and “insect resistance gene” are used interchangeably herein.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Overexpression” refers to the production of a gene product in transgenic organisms that exceeds levels of production in a null segregating (or non-transgenic) organism from the same experiment.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

A “favorable allele” is the allele at a particular locus that confers, or contributes to, a desirable phenotype, e.g., increased cell wall digestibility, or alternatively, is an allele that allows the identification of plants with decreased cell wall digestibility that can be removed from a breeding program or planting (“counterselection”). A favorable allele of a marker is a marker allele that segregates with the favorable phenotype, or alternatively, segregates with the unfavorable plant phenotype, therefore providing the benefit of identifying plants.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

Sequence alignments and percent identity calculations may be determined using a variety of comparison methods designed to detect homologous sequences including, but not limited to, the MEGALIGN® program of the LASERGENER bioinformatics computing suite (DNASTAR® Inc., Madison, WI). Unless stated otherwise, multiple alignment of the sequences provided herein were performed using the Clustal V method of alignment (Higgins and Sharp, CABIOS. 5:151-153 (1989)) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY-5, WINDOW=4 and DIAGONALS SAVED-4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

The present invention includes a polynucleotide comprising: (i) a nucleic acid sequence of at least 90%, 91%, 92%, 93%, 94%, 95%, 96%, 97%, 98%, 99%, or 100% sequence identity, based on the Clustal V method of alignment, when compared to SEQ ID NOS: 4, 8, 13, 19, 52, 53, 56, 57, 58, 101-119, 136-143; or (ii) a full complement of the nucleic acid sequence of (i), wherein the polynucleotide acts as a regulator of gene expression in a plant cell.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

### Regulatory Sequences:

A recombinant DNA construct (including a suppression DNA construct) of the present invention may comprise at least one regulatory sequence.

“Regulatory sequences” or “regulatory elements” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Developmentally regulated promoter” refers to a promoter whose activity is determined by developmental events.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance drought tolerance. This effect has been observed in Arabidopsis (Kasuga et al. (1999) Nature Biotechnol. 17:287-91).

Suitable constitutive promoters for use in a plant host cell include, but are not limited to, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), and the like. Other constitutive promoters include, but are not limited to, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the invention, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present invention which causes the desired temporal and spatial expression.

Promoters which are seed or embryo-specific and may be useful in the invention include, but are not limited to, soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include, but are not limited to, Arabidopsis thaliana 2S seed storage protein gene promoter to express enkephalin peptides in Arabidopsis and Brassica napus seeds (Vanderkerckhove et al., Bio/Technology 7: L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, but are not limited to, for example, promoters regulated by light, heat, stress, flooding or drought, phytohormones, wounding, or chemicals such as ethanol, jasmonate, salicylic acid, or safeners.

For instance, introns of the present invention can be combined with inducible promoters to enhance their activity without affecting their inducibility characteristics.

A minimal or basal promoter is a polynucleotide molecule that is capable of recruiting and binding the basal transcription machinery. One example of basal transcription machinery in eukaryotic cells is the RNA polymerase Il complex and its accessory proteins.

Plant RNA polymerase Il promoters, like those of other higher eukaryotes, are comprised of several distinct “cis-acting transcriptional regulatory elements,” or simply “cis-elements,” each of which appears to confer a different aspect of the overall control of gene expression. Examples of such cis-acting elements include, but are not limited to, such as TATA box and CCAAT or AGGA box. The promoter can roughly be divided in two parts: a proximal part, referred to as the core, and a distal part. The proximal part is believed to be responsible for correctly assembling the RNA polymerase Il complex at the right position and for directing a basal level of transcription, and is also referred to as “minimal promoter” or “basal promoter”. The distal part of the promoter is believed to contain those elements that regulate the spatio-temporal expression. In addition to the proximal and distal parts, other regulatory regions have also been described, that contain enhancer and/or repressors elements The latter elements can be found from a few kilobase pairs upstream from the transcription start site, in the introns, or even at the 3′ side of the genes they regulate (Rombauts, S. et al. (2003) Plant Physiology 132:1162-1176, Nikolov and Burley, (1997) Proc Natl Acad Sci USA 94:15-22), Tjian and Maniatis (1994) Cell 77:5-8; Fessele et al., 2002 Trends Genet 18:60-63, Messing et al., (1983) Genetic Engineering of Plants: an Agricultural Perspective, Plenum Press, NY, pp 211-227).

When operably linked to a heterologous polynucleotide sequence, a promoter controls the transcription of the linked polynucleotide sequence.

In an embodiment of the present invention, the “cis-acting transcriptional regulatory elements” from the promoter sequence disclosed herein can be operably linked to “cis-acting transcriptional regulatory elements” from any heterologous promoter. Such a chimeric promoter molecule can be engineered to have desired regulatory properties. In an embodiment of this invention a fragment of the disclosed promoter sequence that can act either as a cis-regulatory sequence or a distal-regulatory sequence or as an enhancer sequence or a repressor sequence, may be combined with either a cis-regulatory or a distal regulatory or an enhancer sequence or a repressor sequence or any combination of any of these from a heterologous promoter sequence.

In a related embodiment, a cis-element of the disclosed promoter may confer a particular specificity such as conferring enhanced expression of operably linked polynucleotide molecules in certain tissues and therefore is also capable of regulating transcription of operably linked polynucleotide molecules. Consequently, any fragment, portion, or region of the promoter comprising the polynucleotide sequence shown in SEQ ID NO: 105, 106, 107, 108, 109, 110, 111, 112, 113, 114, 115, 116, 117, 119, 136 or 139 can be used as a regulatory polynucleotide molecule.

Promoter fragments that comprise regulatory elements can be added, for example, fused to the 5′ end of, or inserted within, another promoter having its own partial or complete regulatory sequences (Fluhr et al., Science 232:1106-1112, 1986; Ellis et al., EMBO J. 6:11-16, 1987; Strittmatter and Chua, Proc. Nat. Acad. Sci. USA 84:8986-8990, 1987; Poulsen and Chua, Mol. Gen. Genet. 214:16-23, 1988; Comai et al., Plant Mol. Biol. 15:373-381, 1991; 1987; Aryan et al., Mol. Gen. Genet. 225:65-71, 1991).

Cis elements can be identified by a number of techniques, including deletion analysis, i.e., deleting one or more nucleotides from the 5′ end or internal to a promoter; DNA binding protein analysis using DNase I footprinting; methylation interference; electrophoresis mobility-shift assays, in vivo genomic footprinting by ligation-mediated PCR; and other conventional assays; or by sequence similarity with known cis element motifs by conventional sequence comparison methods. The fine structure of a cis element can be further studied by mutagenesis (or substitution) of one or more nucleotides or by other conventional methods (see for example, Methods in Plant Biochemistry and Molecular Biology, Dashek, ed., CRC Press, 1997, pp. 397-422; and Methods in Plant Molecular Biology, Maliga et al., eds., Cold Spring Harbor Press, 1995, pp. 233-300).

Cis elements can be obtained by chemical synthesis or by cloning from promoters that include such elements, and they can be synthesized with additional flanking sequences that contain useful restriction enzyme sites to facilitate subsequent manipulation. Promoter fragments may also comprise other regulatory elements such as enhancer domains, which may further be useful for constructing chimeric molecules.

Methods for construction of chimeric and variant promoters of the present invention include, but are not limited to, combining control elements of different promoters or duplicating portions or regions of a promoter (see for example, U.S. Pat. Nos. 4,990,607; 5,110,732; and 5,097,025). Those of skill in the art are familiar with the standard resource materials that describe specific conditions and procedures for the construction, manipulation, and isolation of macromolecules (e.g., polynucleotide molecules and plasmids), as well as the generation of recombinant organisms and the screening and isolation of polynucleotide molecules.

In an embodiment of the present invention, the promoters disclosed herein can be modified. Those skilled in the art can create promoters that have variations in the polynucleotide sequence. The polynucleotide sequence of the promoters of the present invention as shown in SEQ ID NOS: 105-113, 119, 136 or 139, may be modified or altered to enhance their control characteristics. As one of ordinary skill in the art will appreciate, modification or alteration of the promoter sequence can also be made without substantially affecting the promoter function. The methods are well known to those of skill in the art. Sequences can be modified, for example by insertion, deletion, or replacement of template sequences in a PCR-based DNA modification approach.

The present invention encompasses functional fragments and variants of the promoter sequences disclosed herein.

A “functional fragment” of a regulatory sequence herein is defined as any subset of contiguous nucleotides of any of the regulatory sequences disclosed herein, that can perform the same, or substantially similar function as the full length promoter sequences disclosed herein.

A “functional fragment of a promoter” with substantially similar function to a full length promoter disclosed herein refers to a functional fragment that retains largely the same level of activity as the full length promoter sequence and exhibits the same pattern of expression as the full length promoter sequence.

A “variant promoter”, as used herein, is the sequence of the promoter or the sequence of a functional fragment of a promoter containing changes in which one or more nucleotides of the original sequence is deleted, added, and/or substituted, while substantially maintaining promoter function. One or more base pairs can be inserted, deleted, or substituted internally to a promoter. In the case of a promoter fragment, variant promoters can include changes affecting the transcription of a minimal promoter to which it is operably linked. Variant promoters can be produced, for example, by standard DNA mutagenesis techniques or by chemically synthesizing the variant promoter or a portion thereof.

Enhancer sequences refer to the sequences that can increase gene expression. These sequences can be located upstream, within introns or downstream of the transcribed region. The transcribed region is comprised of the exons and the intervening introns, from the promoter to the transcription termination region. The enhancement of gene expression can be through various mechanisms which include, but are not limited to, increasing transcriptional efficiency, stabilization of mature mRNA and translational enhancement.

Recombinant DNA constructs of the present invention may also include other regulatory sequences, including but not limited to, translation leader sequences, introns, and polyadenylation recognition sequences. In another embodiment of the present invention, a recombinant DNA construct of the present invention further comprises an enhancer or silencer.

An “intron” is an intervening sequence in a gene that is transcribed into RNA and then excised in the process of generating the mature mRNA. The term is also used for the excised RNA sequences. An “exon” is a portion of the sequence of a gene that is transcribed and is found in the mature messenger RNA derived from the gene, and is not necessarily a part of the sequence that encodes the final gene product.

Many genes exhibit enhanced expression on inclusion of an intron in the transcribed region, especially when the intron is present within the first 1 kb of the transcription start site. The increase in gene expression by presence of an intron can be at both the mRNA (transcript abundance) and protein levels. The mechanism of this Intron Mediated Enhancement (IME) in plants is not very well known (Rose et al., Plant Cell, 20:543-551 (2008) Le-Hir et al, Trends Biochem Sci . . . 28:215-220 (2003), Buchman and Berg, Mol. Cell Biol. (1988) 8:4395-4405; Callis et al., Genes Dev. 1 (1987): 1183-1200).

An “enhancing intron” is an intronic sequence present within the transcribed region of a gene which is capable of enhancing expression of the gene when compared to an intronless version of an otherwise identical gene. An enhancing intronic sequence might also be able to act as an enhancer when located outside the transcribed region of a gene, and can act as a regulator of gene expression independent of position or orientation (Chan et. al. (1999) Proc. Natl. Acad. Sci. 96:4627-4632; Flodby et al. (2007) Biochem. Biophys. Res. Commun. 356:26-31).

Short consensus sequences or motifs can be identified from the intron sequences experimentally identified to be enhancing introns. These motifs can be used to scan and help identify more gene-expression enhancing introns. A motif capable of conferring transgene expression in male reproductive tissue in dicot plants has been described in US application No. US2007/020436.

An 8-bp sequence (SEQ ID NO: 99) and a 5-bp sequence (SEQ ID NO: 100) that can be used for identifying novel enhancing introns have been described in this application. Some variations of the 8-bp sequence can also be useful for identifying enhancing introns. The useful variations from the 8-bp motif (SEQ ID NO: 99) described herein can occur mainly at the first three positions. The last 5 bp of the sequence are highly conserved. Also, the variations from the 8-bp consensus (SEQ ID NO: 99) occur at maximum two out of 8 positions at any one time. In the event of more than 2 bp being different than the consensus, the enhancing intron might have additional copies of either the 5-bp (SEQ ID NO: 100) or the 8-bp motif (SEQ ID NO: 99).

The motif variations can be represented as a consensus motif sequence, Y[R/T]RATCYG (SEQ ID NO: 146). The first position can be any of the two pyrimidine bases, C or T. The second position can be substituted by an A, G or T. The third position can be a purine. The ATC core is the most highly conserved region, and does not exhibit any variability.

An intron sequence can be added to the 5′ untranslated region, the protein-coding region or the 3′ untranslated region to increase the amount of the mature message that accumulates in the cytosol.

The intron sequences can be operably linked to a promoter. Promoters may be derived in their entirety from a native gene, or be composed of different elements derived from different promoters found in nature, or even comprise synthetic DNA segments.

Sequences orthologous to an intron are sequences that are present in orthologous genes at the same position as the intron in the original gene sequence.

The tissue expression patterns of the genes can be determined using the RNA profile database of the Massively Parallel Signature Sequencing (MPSS™). This proprietary database contains deep RNA profiles of more than 250 libraries and from a broad set of tissue types. The MPSS™ transcript profiling technology is a quantitative expression analysis that typically involves 1-2 million transcripts per cDNA library (Brenner S. et al., (2000). Nat Biotechnol 18:630-634, Brenner S. et al. (2000) Proc Natl Acad Sci USA 97:1665-1670). It produces a 17-base high quality usually gene-specific sequence tag usually captured from the 3′-most Dpnll restriction site in the transcript for each expressed gene. The use of this MPSS data including statistical analyses, replications, etc, has been described previously (Guo M et al. (2008) Plant Mol Biol 66:551-563).

IMEter is a word-based discriminator that can do a computational analysis as to whether an intron can act as an enhancer of gene expression or not. The IMeter scoring system is described in Rose, A. B. (2004). Plant J. 40_744-751, and Rose et al (2008) Plant Cell 20:543-551.

“Transcription terminator”, “termination sequences”, or “terminator” as described herein refer to DNA sequences located downstream of a coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al., Plant Cell 1:671-680 (1989). A polynucleotide sequence with “terminator activity” refers to a polynucleotide sequence that, when operably linked to the 3′ end of a second polynucleotide sequence that is to be expressed, is capable of terminating transcription from the second polynucleotide sequence and facilitating efficient 3′ end processing of the messenger RNA resulting in addition of poly A tail. Transcription termination is the process by which RNA synthesis by RNA polymerase is stopped and both the processed messenger RNA and the enzyme are released from the DNA template.

Improper termination of an RNA transcript can affect the stability of the RNA, and hence can affect protein expression. Variability of transgene expression is sometimes attributed to variability of termination efficiency (Bieri et al (2002) Molecular Breeding 10:107-117). As used herein, the terms “bidirectional transcriptional terminator” and “bidirectional terminator” refer to a transcription terminator sequence that has the capability of terminating transcription in both 5′ to 3′, and 3′ to 5′ orientations. A single sequence element that acts as a bidirectional transcriptional terminator can terminate transcription from two convergent genes.

The present invention encompasses functional fragments and variants of the terminator sequences disclosed herein.

A “functional fragment of a terminator” with substantially similar function to the full length terminator disclosed herein refers to a functional fragment that retains the ability to terminate transcription largely to the same level as the full length terminator sequence. A recombinant construct comprising a heterologous polynucleotide operably linked to a “functional fragment” of the terminator sequence disclosed herein exhibits levels of heterologous polynucleotide expression substantially similar to a recombinant construct comprising a heterologous polynucleotide operably linked to the full length terminator sequence.

A “variant terminator”, as used herein, is the sequence of the terminator or the sequence of a functional fragment of a terminator containing changes in which one or more nucleotides of the original sequence is deleted, added, and/or substituted, while substantially maintaining terminator function. One or more base pairs can be inserted, deleted, or substituted internally to a terminator, without affecting its activity. Fragments and variants can be obtained via methods such as site-directed mutagenesis and synthetic construction.

These terminator functional fragments will comprise at least about 20 contiguous nucleotides, preferably at least about 50 contiguous nucleotides, more preferably at least about 75 contiguous nucleotides, even more preferably at least about 100 contiguous nucleotides of the particular terminator nucleotide sequence disclosed herein. Such fragments may be obtained by use of restriction enzymes to cleave the naturally occurring terminator nucleotide sequences disclosed herein; by synthesizing a nucleotide sequence from the naturally occurring terminator DNA sequence; or may be obtained through the use of PCR technology. See particularly, Mullis et al., Methods Enzymol. 155:335-350 (1987), and Higuchi, R. In PCR Technology: Principles and Applications for DNA Amplifications; Erlich, H. A., Ed.; Stockton Press Inc.: New York, 1989. Again, variants of these terminator fragments, such as those resulting from site-directed mutagenesis, are encompassed by the compositions of the present invention.

The terms “substantially similar” and “corresponding substantially” as used herein refer to nucleic acid fragments, particularly regulatory sequences, wherein changes in one or more nucleotide bases do not substantially alter the ability of the regulatory sequence to perform the same function as the corresponding full length sequence disclosed herein. These terms also refer to modifications, including deletions and variants, of the nucleic acid sequences of the instant invention by way of deletion or insertion of one or more nucleotides that do not substantially alter the functional properties of the resulting sequence relative to the initial, unmodified sequence. It is therefore understood, as those skilled in the art will appreciate, that the invention encompasses more than the specific exemplary sequences.

As will be evident to one of skill in the art, any heterologous polynucleotide of interest can be operably linked to the regulatory sequences described in the current invention. Examples of polynucleotides of interest that can be operably linked to the regulatory sequences described in this invention include, but are not limited to, polynucleotides comprising other regulatory elements such as introns, enhancers, promoters, translation leader sequences, protein coding regions such as disease and insect resistance genes, genes conferring nutritional value, genes conferring yield and heterosis increase, genes that confer male and/or female sterility, antifungal, antibacterial or antiviral genes, and the like. Likewise, the regulatory sequences described in the current invention can be used to regulate transcription of any nucleic acid that controls gene expression. Examples of nucleic acids that could be used to control gene expression include, but are not limited to, antisense oligonucleotides, suppression DNA constructs, or nucleic acids encoding transcription factors.

Embodiments of the invention are:

The present invention relates to regulatory sequences for modulating gene expression in plants. Recombinant DNA constructs comprising regulatory sequences are provided. Recombinant DNA constructs comprising intron sequences acting as enhancers of gene expression and endogenous promoter and terminator sequences corresponding to these intron sequences are provided.

Another embodiment of the invention is a recombinant DNA construct comprising an intron operably linked to a promoter and a terminator wherein the intron comprises a nucleotide sequence that has at least 95% sequence identity to SEQ ID NO: 4, 8, 13, 19, 52, 53, 56, 57, 58, 101, 102, 103, 104, 118, 137 or 138. In another embodiment, the intron comprises the nucleotide sequence of SEQ ID NO: 4, 8, 13, 19, 52, 53, 56, 57, 58, 101, 102, 103, 104, 118, 137 or 138.

One embodiment of the invention is a recombinant DNA construct comprising an intron operably linked to a promoter and a terminator wherein the promoter comprises a nucleotide sequence that has at least 95% sequence identity to SEQ ID NO: 105-117, 119, 136 or 139. In another embodiment, the promoter comprises the nucleotide sequence of SEQ ID NO: 105-117, 119, 136 or 139.

One embodiment of the invention is a recombinant DNA construct comprising an intron operably linked to a promoter and a terminator wherein the terminator comprises a nucleotide sequence that has at least 95% sequence identity to SEQ ID NOS: 140, 141, 142 or 143. In another embodiment, the terminator comprises the nucleotide sequence of SEQ ID NO: 140, 141, 142 or 143.

One embodiment of the invention is a recombinant DNA construct comprising an intron operably linked to a promoter and a terminator wherein the intron comprises a nucleotide sequence that has at least 95% identity to SEQ ID NOS: 4, 8, 13, 19, 52, 53, 56, 57, 58, 101, 102, 103, 104, 118, 137 or 138; and the promoter comprises a nucleotide sequence that has at least 95% identity to SEQ ID NOS: 105, 106, 107, 108, 109, 110, 111, 112, 113, 114, 115, 116, 117, 119, 136 or 139.

One embodiment of the invention is a recombinant DNA construct comprising an intron operably linked to a promoter and a terminator wherein the intron comprises a nucleotide sequence that has at least 95% identity to SEQ ID NO: 4, 8, 13, 19, 52, 53, 56, 57, 58, 101, 102, 103, 104, 118, 137 or 138; the promoter sequence has at least 95% identity to SEQ ID NO: 105, 106, 107, 108, 109, 110, 111, 112, 113, 114, 115, 116, 117, 119, 136 or 139; and the terminator has at least 95% sequence identity to SEQ ID NO: 140, 141, 142 or 143.

In one embodiment of the current invention, the intron is operably linked to the promoter, and is present downstream of the promoter, in the recombinant DNA constructs described herein. One embodiment of the present invention includes a recombinant DNA construct comprising an intron described in the present invention, operably linked to a promoter and a heterologous polynucleotide, wherein the intron can act as enhancer of expression of the heterologous polynucleotide.

Another embodiment of the invention encompasses a recombinant DNA construct comprising an intron wherein the intron sequence comprises at least one copy of the 8-bp sequence motif of SEQ ID NO. 99; or contains at least one copy of the 8-bp sequence motif of SEQ ID NO: 99 and at least one copy of the 5-bp sequence motif of SEQ ID NO: 100, wherein the intron is capable of enhancing expression of a heterologous polynucleotide in a transgenic plant. The intron sequence can also comprise more than one copy of SEQ ID NO: 99, or can comprise one or more than one copy of SEQ ID NO: 99 and more than one copy of SEQ ID NO: 100.

Another embodiment of this invention is a method to identify novel introns that are useful for enhancing expression of a heterologous polynucleotide in a plant cell, the method comprising the steps of scanning a plurality of introns from plants for presence of SEQ ID NO: 99, selecting a sequence that contains at least one copy of SEQ ID NO: 99, measuring the efficacy of the identified intron to enhance expression of a heterologous polynucleotide in a plant.

Another embodiment of the invention is a method for identifying novel intronic sequences for enhancing transgene expression in monocotyledenous plants by identifying sequences orthologous to SEQ ID NO: 4, 8, 13, 19, 52, 53, 56, 57, 58, 101, 102, 103, 104, 118, 137 or 138; and measuring the enhancing effect of the identified intron on the expression of an operably linked heterologous polynucleotide.

Another embodiment of the current invention includes the promoter and the terminator sequences that are endogenously linked to the introns identified using the methods described in the current invention.

Another embodiment of the current invention is a method for modulating expression of a heterologous polynucleotide in a monocotyledonous plant comprising the steps of: (a) introducing into a regenerable plant cell a recombinant DNA construct comprising a promoter and a heterologous polynucleotide wherein each is operably linked to an intron, wherein the intron comprises either (i) a nucleotide sequence that is orthologous to SEQ ID NO: 4, 8, 13, 19, 52, 53, 56, 57, 58, 101, 102, 103, 104, 118, 137 or 138; or (ii) a nucleotide sequence that contains least one copy of a sequence motif identical to SEQ ID NO: 99; and; (b) regenerating a transgenic plant from a regenerable monocotyledonous plant cell after step (a) wherein the transgenic plant comprises the recombinant DNA construct; and (c) obtaining a progeny plant derived from the transgenic plant of step (b), wherein said progeny plant comprises the recombinant DNA construct and exhibits enhanced expression of the heterologous polynucleotide when compared to a plant comprising a corresponding recombinant DNA construct without the intron sequence.

In another embodiment, this invention concerns a vector, cell, plant, or seed comprising a recombinant DNA construct comprising the regulatory sequences described in the present invention.

The invention encompasses regenerated, mature and fertile transgenic plants comprising the recombinant DNA constructs described above, transgenic seeds produced therefrom, T1 and subsequent generations. The transgenic plant cells, tissues, plants, and seeds may comprise at least one recombinant DNA construct of interest.

In one embodiment, the plant comprising the regulatory sequences described in the present invention is a monocotyledenous plant. In another embodiment, the plant comprising the regulatory sequences described in the present invention is a maize plant.

### EXAMPLES

The present invention is further illustrated in the following Examples, in which parts and percentages are by weight and degrees are Celsius, unless otherwise stated. It should be understood that these examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Furthermore, various modifications of the invention in addition to those shown and described herein will be apparent to those skilled in the art from the foregoing description. Such modifications are also intended to fall within the scope of the appended claims.

### Example 1

### Identification of Candidate Gene Expression/Transcript-Enhancing First Introns

Introns that may enhance transcript abundance were sought from among a set of maize genes which (a) had first introns near the N-terminus of the transcript, and (b) had high level transcript abundance. A subset of maize genes were identified whose models were deemed to be complete. This assessment was done using a combination of maize public B73 BAC sequences plus a proprietary EST transcript assembly in an analysis comparing the predicted gene structures and the predicted transcript open reading frames (ORFs) in relation to public reference proteins plus some manual curations. Only full-length transcripts were considered; that is, those with complete protein coding regions. This set did not represent all maize genes, and there was some redundancy in the list.

This set of gene models was then analyzed versus a body of over 250 MPSS mRNA transcript profiling samples produced from a variety of maize tissues and treatments. The MPSS profiling technology produces a 17-bp tag sequence beginning with GATC. These tags were matched to the gene set via the full-length transcript, and those genes which (a) had an MPSS tag matching the plus strand of the transcript, and (b) had a measured expression level of at least 1000 ppm (parts per million) in at least one of the MPSS samples, were retained. In this way a working set of 3131 genes was produced. Using the maize BAC genomic sequence to analyze these 3131 genes, a subset of genes was produced that (a) contained an intron, and (b) contained an intron which was located within the 5′UTR or within the first 300 nucleotides of the ORF. This resulted in a subset of 1185 genes for further consideration.

This set of 1185 candidate genes was then filtered down by a number of criteria. Duplicates were removed. Introns without canonical GT-AG rules were excluded. Genes whose expression was defined by ‘promiscuous’ MPSS tags, such as GATCAAAAAAAAAAAAA (SEQ ID NO: 145), and also MPSS tags matching repetitive elements, were removed. Genes whose first introns were greater than 2 kb were dropped. In addition, genes whose first introns' GC content were higher than 50% GC and/or the intron T (=U) content was below 25% were removed. In addition, the IMeter score for the first intron had to be positive. The IMeter scoring system is described in Rose, A. B. (2004) Plant J. 40:744-751. This resulted in an interim set of remaining 331 candidates. This set was then further manually winnowed down to 86 by positively considering a combinations of factors but chiefly: (a) the breadth of diverse tissue expression and (b) the ratio of the IMeter score to intron length.

This set of 86 introns was one prioritized pool from which introns were drawn for functional testing of whether they enhance transcript abundance. Seventeen of these 86 were tested.

### Example 2

Creation of an Intron Testing Vector with Maize Ubiquitin Promoter

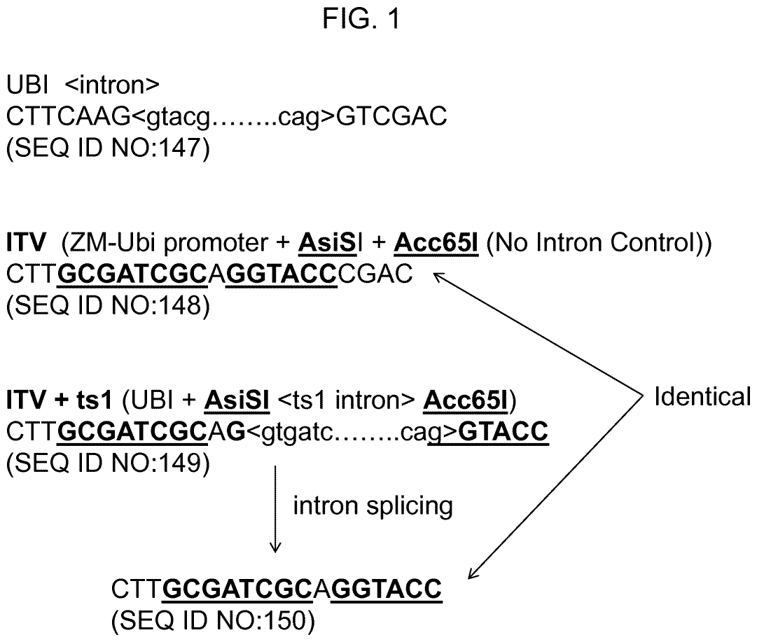

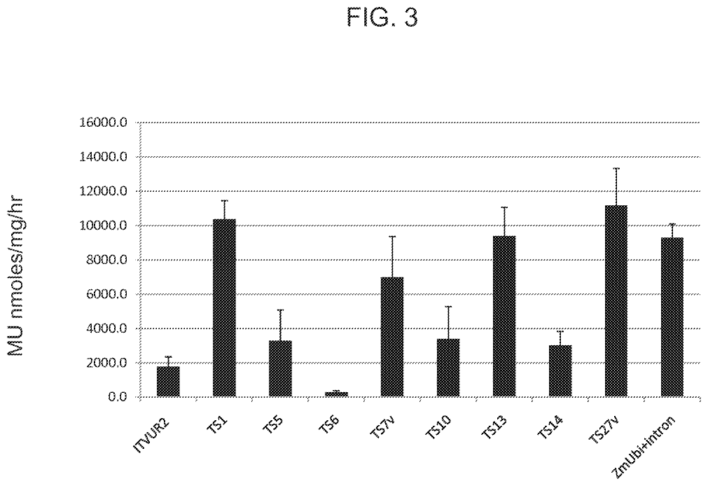

Maize ubi promoter (SEQ ID NO: 1) along with its intron (SEQ ID NO: 2) in the 5′ UTR confers high level constitutive expression in monocot plants (Christensen, A. H., Sharrock, R. A. and Quail, P. H., Plant Mol. Biol. 18, 675-89, 1992). This high-level expression is dependent on the first intron in the 5′ UTR. Removal of this intron results in a >4-fold reduction in expression measured by transient assays (FIG. 3). We created a plant transformation vector where the maize ubiquitin promoter together with its endogenous intron drives E. coli β-glucuronidase (GUS) reporter gene expression. We then replaced the maize ubiquitin intron with two restriction sites, AsiS1 and Acc651 to allow the insertion of novel introns and test their ability to enhance reporter gene expression driven by the ubiquitin promoter (SEQ ID NO: 1) (FIG. 1).

### Example 3

### Intron Amplification and Cloning

Zea mays B73 seeds were germinated in Petri plates and genomic DNA was made from seedling leaf tissue using the QIAGEN® DNEASY® Plant Maxi Kit (QIAGEN® Inc.) according to the manufacturer's instructions. DNA products were amplified with primers shown in Table 2 using genomic DNA as template with PHUSION™ DNA polymerase (New England Biolabs Inc.). The resulting DNA fragments were cloned into the intron testing vector ITVUR-2 (SEQ ID NO: 3), using standard molecular biology techniques (Sambrook et al.) or using INFUSION™ from (Clontech Inc.), and sequenced completely.

TABLE 2

| Intron | | | Forward Primer | Reverse Primer |
|---|---|---|---|---|
| Name | SEQ ID NO | Length (nt) | (SEQ ID NO) | (SEQ ID NO) |
| TS1 | 4 | 814 | 20 | 21 |
| TS4 | 5 | 727 | 22 | 23 |
| TS5 | 6 | 834 | 24 | 25 |
| TS6 | 7 | 982 | 26 | 27 |
| TS7v | 137 | 856 | 28 | 29 |
| TS8 | 9 | 1020 | 30 | 31 |
| TS10 | 10 | 841 | 32 | 33 |
| TS11 | 11 | 1044 | 34 | 35 |
| TS12 | 12 | 648 | 36 | 37 |
| TS13 | 13 | 632 | 38 | 39 |
| TS14 | 14 | 1405 | 40 | 41 |
| TS15 | 15 | 1361 | 42 | 43 |
| TS16 | 16 | 703 | 44 | 45 |
| TS17 | 17 | 1341 | 46 | 47 |
| TS24 | 18 | 1125 | 48 | 49 |
| TS27v | 138 | 884 | 50 | 51 |

All the constructs were mobilized into the Agrobacterium strain LBA4404/pSB1 and selected on Spectinomycin and tetracycline. Agrobacterium transformants were isolated and the integrity of the plasmid was confirmed by retransforming to E. coli or PCR analysis.

### Example 4

Transient Transformation and Expression of Intron Constructs in Maize Embryos Infected with Agrobacterium

### Preparation of Agrobacterium Suspension:

Agrobacterium was streaked out from −80° C. frozen aliquot onto a plate containing PHI-L medium and was cultured at 28° C. in the dark for 2 days. The PHI-L medium comprises 50 ml Stock Solution A, 50 ml/L stock Solution B, 900 ml Stock Solution C and spectinomycin (Sigma chemicals) was added to a concentration of 50 mg/L in sterile ddH2O (Stock Solution A: K2HPO4 60 g/l, NaH2PO4 20 g/l, pH adjusted to 7.0 w/KOH and autoclaved; stock solution B: NH4Cl 20 g/l, MgSO4·7H2O 6 g/l, KCl 3 g/l, CaCl2 0.2 g/l, FeSO4·7H2O 50 mg/l; stock solution C: glucose 5 g/l, agar 15 g/l (#A-7049, Sigma Chemicals, St. Louis, Mo.) and was autoclaved.

The plate can be stored at 4° C. and used usually for about 1 month. A single colony was picked from the master plate and was streaked onto a plate containing PHI-M medium [Yeast Extract 5 g/l (Difco); Peptone 10 g/l (Difco); NaCl 5 g/l (Hi-Media); agar (Sigma Chemicals) 15 g/l; pH 6.8, containing 50 mg/l spectinomycin] and incubated at 28° C. in the dark for overnight.

Five ml of PHI-A, [CHU (N6) Basal salts (Sigma C-1416) 4 g/l; Erikson's vitamin solution (1000×, Sigma-1511) 1 ml/l; Thiamine·HCl (Sigma) 0.5 mg/l; 2,4-Dichloro phenoxyacetic acid (2,4-D, Sigma) 1.5 mg/l; L-Proline (Sigma) 0.69 g/l; Sucrose (Sigma) 68.5 g/l; Glucose (Sigma) 36 g/l; pH adjusted to 5.2 with KOH] was added to a 14 ml FALCON™ tube in a hood. About 3 full loops (5 mm loop size) Agrobacterium was collected from the plate and suspended in the tube, then the tube vortexed to make an even suspension. One ml of the suspension was transferred to a spectrophotometer tube and the OD of the suspension was adjusted to 0.72 at 550 nm by adding either more Agrobacterium or more of the same suspension medium, for an Agrobacterium concentration of approximately 0.5×10⁹ cfu/ml. The final Agrobacterium suspension was aliquoted into 2 ml microcentrifuge tubes, each containing 1 ml of the suspension. The suspension was then used as soon as possible.

### Embryo Isolation, Infection and Co-Cultivation:

About 2 ml of the same medium (PHI-A) which is used for the Agrobacterium suspension was added into a 2 ml microcentrifuge tube. Immature embryos were isolated from a sterilized ear with a sterile spatula and dropped directly into the medium in the tube. A total of 25 embryos are placed in the tube. The optimal size of the embryos was about 1.7-2.0 mm. The entire medium was drawn off and 1 ml of Agrobacterium suspension was added to the embryos and the tube was vortexed for 30 sec. The tube was allowed to stand for 5 min in the hood. The suspension of Agrobacterium and embryos was poured into a Petri plate containing co-cultivation medium PHI-B [CHU (N6) Basal salts (Sigma C-1416) 4 g/l; Eriksson's vitamin solution (1000×, Sigma-1511) 1 ml/l; Thiamine·HCl 0.5 mg/l; 2,4-D 1.5 mg/l; L-Proline 0.69 g/l; GELRITE® (Sigma) 3 g/l; Sucrose 30 g/l; pH adjusted to 5.8 with KOH; Post sterilization, Silver nitrate (0.85 mg/l) and acetosyringone (100 mM) were added after cooling the medium to 45° C.]. Any embryos left in the tube were transferred to the plate using a sterile spatula. The Agrobacterium suspension was drawn off and the embryos placed axis side down on the media. The plate was sealed with PARAFILM® and was incubated in the dark at 23-25° C. for about 3 days of co-cultivation.

### Resting of Co-Cultivated Embryos:

For the resting step, all the embryos were transferred to a new plate containing PHI-C medium [CHU (N6) Basal salts (Sigma C-1416) 4 g/l; Eriksson's vitamin solution (1000×, Sigma-1511) 1 ml/l; Thiamine·HCl 0.5 mg/l; 2,4-D 1.5 mg/l; L-Proline 0.69 g/l; Sucrose 30 g/l; MES buffer (Sigma) 0.5 g/l; agar (Sigma 1-7049) 8 g/l; pH adjusted to 5.8 with KOH; Post sterilization, Silver nitrate (0.85 mg/l) and carbenicillin (100 mg/l) were added after cooling the medium to 45° C.]. The plates were sealed with PARAFILM® and incubated in the dark at 28° C. for 3-5 days.

### Histochemical and Fluorometric GUS Analysis:

Transformed embryos were taken for expression analysis after 3 days of resting. Ten embryos for each construct were used for histochemical GUS staining using standard protocols (Janssen and Gardner, Plant Mol. Biol. (1989) 14:61-72,) and two pools of 5 each were used to do quantitative assays using MUG substrate using standard protocols [Jefferson, R. A., Nature. 342:837-838 (1989); Jefferson, R. A., Kavanagh, T. A. & Bevan, M. W. EMBO J. 6:3901-3907 (1987)] (FIG. 3). Introns TS1 (SEQ ID NO: 4), TS7v (SEQ ID NO: 137), TS13 (SEQ ID NO: 13) and TS27v (SEQ ID NO: 138) all enhanced the GUS reporter gene expression between 3 to 5 fold when compared to the ubiquitin promoter alone without any intron. The level of enhancement is comparable to that of the maize ubiquitin first intron. Introns TS4, TS5, TS6, TS8, TS10, TS11, TS12, TS14, TS15, TS16, TS17 and TS24 did not enhance expression (Data shown for TS5, TS6, TS10 and TS14 in FIG. 3).

### Example 5

### Transient Transformation and Expression of Intron Constructs in Rice Calli via Agrobacterium

### Preparation of Agrobacterium Suspension:

Agrobacterium was streaked out from −80° C. frozen aliquot onto a plate containing YEB medium and was cultured at 28° C. in the dark for 2 days. The YEB medium comprises (MgSO4 (Hi-Media) 0.2 g/l; K2HPO4 (Fisher Scientific) 0.5 g/l; Mannitol 10 g/l; NaCl 0.1 g/l; Yeast Extract 0.4 g/l; Agar 15 g/l). Agrobacterium cultures harboring the intron constructs were cultured one day prior to rice calli infection in YEB broth. A large swipe of Agrobacterium growth was inoculated into 7.5 ml of YEB broth in FALCON™ tubes. Then in the next morning OD of each culture was measured at 550 nm. Cultures were centrifuged at 4000 rpm for 10 minutes. Supernatant was discarded and the pellet was resuspended in PHI-L supplemented with Acetosyringone at 100 μM. Another spin was given to Agrobacterium cultures at 4000 rpm for 10 min and the pellets were resuspended in PHI-L supplemented with Acetosyringone at 100UM and the OD was adjusted to 1.0 by adding either more Agrobacterium or more of the same suspension medium, for an Agrobacterium concentration of approximately 0.5×10⁹ cfu/ml.

### Rice Callus Induction, Infection and Co-Cultivation:

15 to 21 days old Rice calli which were grown on callus induction medium, PHI-R [CHU (N6) Basal salts (Sigma C-1416) 4 g/l; Eriksson's vitamin solution (1000×, Sigma-1511) 1 ml/l; Thiamine·HCl 0.5 mg/l; 2,4-D 2.0 mg/l; L-Proline 0.69 g/l; Casein hydrolysate (Sigma) 300 mg/l; Sucrose (Sigma) 30 g/l; GELRITE® (Sigma) 4 g/l; pH adjusted to 5.8 with KOH]. Coleoptile of the rice calli was removed and calli were spliced to the size of approximately 2 to 3 mm. Spliced calli were transferred to the FALCON™ tubes containing Agrobacterium cultures and infected for 15 minutes with gentle intermittent shaking. The liquid Agrobacterium culture was decanted and the wet calli were taken out and blotted on sterile WHATMAN® filter paper No 4. Subsequently, the calli were transferred onto co-cultivation medium, PHI-R supplemented with Acetosyringone (Sigma) at 100 μM. The infected calli were co-cultivated in dark at 21° C. for 72 hours.

### Resting of Co-Cultivated Rice Calli:

The co-cultivation was terminated by washing in sterile water containing carbenicillin (Sigma, 400 mg/l). Calli were washed with gentle intermittent shaking in the antibiotic solution for 15 minutes. The wet calli were blotted on WHATMAN® filter paper No 4. The dried calli were transferred to resting/callusing medium, PHI-R in which carbenicillin (400 mg/l) was added after cooling the medium to 45° C. after sterilization. The plates were sealed with PARAFILM® and incubated in the dark at 28° C. for 3-5 days.

### Histochemical and Fluorometric GUS Analysis:

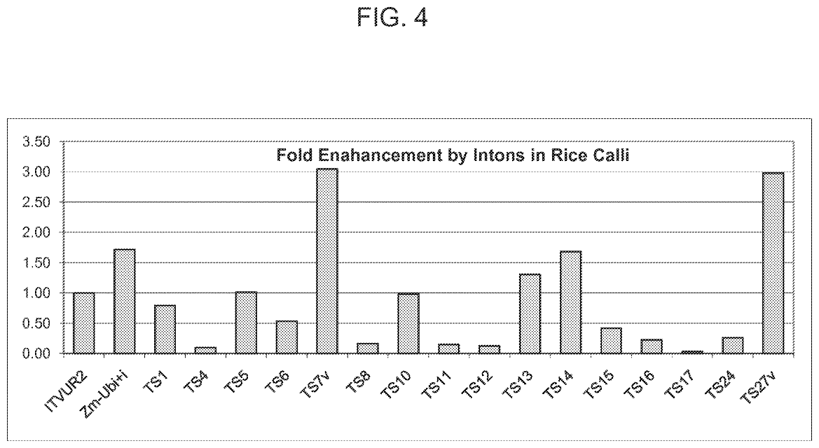

After 3 days, calli were taken for expression analysis. For each construct 20 calli were infected and 8 calli were used for histochemical GUS staining using X-Gluc solution and another eight calli were taken for GUS quantitation using standard protocol (Jefferson et al., EMBO J. 6:3901-3907, 1987). TS7v (SEQ ID NO: 137) and TS27v (SEQ ID NO: 138) were able to enhance GUS reporter expression from the maize ubiquitin promoter (SEQ ID NO: 1) (FIG. 4).

### Example 6

### Description of Constitutive Promoter Selection Via MPSS Samples

Promoter candidates were identified using a set of 241 proprietary expression profiling experiments run on the MPSS (Massively Parallel Signature Sequencing) technology platform provided by Lynx Therapeutics. The 241 samples from corn consisted of various tissue samples spanning most of the range of corn tissues and developmental stages. Each experiment resulted in approximately 20,000 unique sequence tags of 17 bp length from a single tissue sample. Typically these tags could be matched to one or a few transcript sequences from the proprietary “Unicorn” EST assembly set. A query of the MPSS database was performed looking for tags that were observed in 240 or more of the 241 samples. We identified 111 tags that met the criteria and chose 22 that were observed at an expression level of 1 or greater PPM (Parts Per Million tags) in all 241 experiments for further development. 21 of these 22 tags mapped to a single gene based on the transcript set. We took the top 6 candidates from this list and identified the 1500 bp of promoter regions and the first intron, defined as the first intron in the transcript from the 5′ end, (i1 (SEQ ID NO: 52), i2 (SEQ ID NO: 53), i3 (SEQ ID NO: 54), i5 (SEQ ID NO: 56), i6 (SEQ ID NO: 57) and i7 (SEQ ID NO: 58). In addition we also included one second intron (i4; SEQ ID NO: 55) to the list. All introns were evaluated for intron-mediated enhancement of expression from CYMV promoter.

### Example 7

### Enhancement Activity of Introns in Transient Expression System

To determine whether the experimental introns function to enhance promoter activity in plant tissue, transient infiltration assays using the maize suspension cell line, BMS (Black Mexican Sweet), were performed. These Agrobacterium-mediated assays, known in the art, provide a rapid screening method to evaluate the enhancement capability of the introns.

The introns were cloned into an expression vector downstream of the Citrus Yellow Mosaic virus promoter and upstream of the coding region of an insecticidal gene described in US2007/0202089 A1. The insecticidal gene acted as a reporter for expression. A vector with no intron between the promoter and coding region was included to provide a baseline control for expression. A vector (SEQ ID NO: 59; PHP38808) with the Adh1 intron1 was also included to provide a comparison for the level of increased expression by each experimental intron. The Adh1 intron has been shown to enhance the expression of foreign genes in plant tissue (Callis et al. (1987) Genes and Development: 1183-1200; Kyozuka et al. (1990) Maydica 35:353-357). Each expression vector also contained an expression cassette for phosphinothricin acetyl transferase (PAT).

Transiently transformed BMS cells were evaluated for expression by both northern blot analysis for RNA accumulation and ELISA analysis for protein accumulation. If the experimental introns, particularly introns i1 (SEQ ID NO: 52), i2 (SEQ ID NO: 53), i5 (SEQ ID NO: 56), i6 (SEQ ID NO: 57), and i7 (SEQ ID NO: 58), exhibited intron mediated enhancement of expression, the increased expression would be reflected at both the RNA and protein levels.

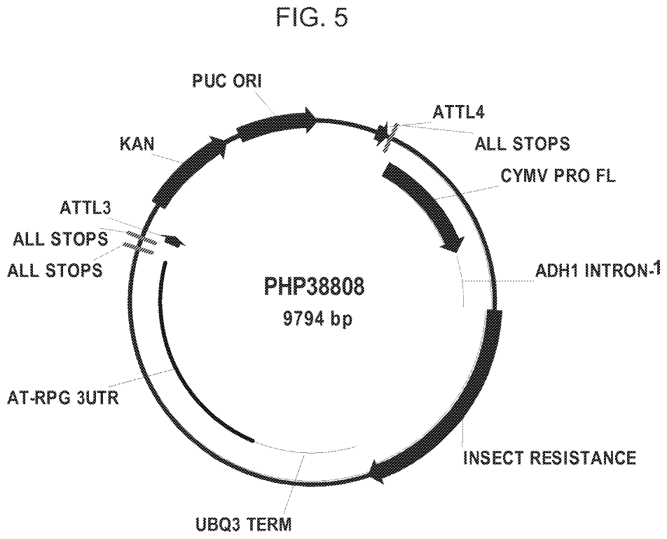

The ratio of expression for each intron cassette showed that introns i1, 12, i5, i6, and i7 had expression levels that were between 2.3 and 4.8 fold higher than the intronless control (Table 3). These increased expression levels were comparable to the control cassette (SEQ ID NO: 59, PHP38808; FIG. 5) containing the Adh1 intron. The ELISA values were standardized for differences in transformation efficiency between vectors by normalizing against PAT gene expression.

TABLE 3
ELISA Results Indicating Expression Levels of Insecticidal Gene
(IG) and PAT in Constructs Containing Experimental Introns

| | IG | PAT | IG/ | Fold difference |
|---|---|---|---|---|
| Intron | (ppm) | (ppm) | PAT | from no intron |
| none | 38.8 | 179.0 | 0.22 | N/A |
| ADH1 | 104.3 | 117.4 | 0.89 | 4.05 |
| i1 | 98.3 | 136.5 | 0.72 | 3.27 |
| i2 | 118.7 | 154.0 | 0.77 | 3.50 |
| i5 | 115.5 | 108.5 | 1.06 | 4.82 |
| i6 | 107.6 | 209.0 | 0.51 | 2.32 |
| i7 | 104.3 | 117.4 | 0.89 | 4.05 |

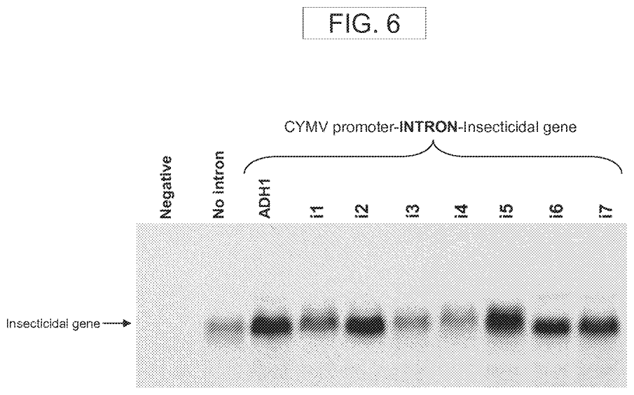

To determine whether introns i1 (SEQ ID NO: 52), i2 (SEQ ID NO: 53), i5 (SEQ ID NO: 56), i6 (SEQ ID NO: 57), and i7 (SEQ ID NO: 58) resulted in increased mRNA levels, northern blot analysis was performed. RNA amounts for each vector were normalized against PAT expression prior to electrophoresis. The results of the analysis mirrored the ELISA results. Introns i1, 12, i5, i6, and i7 facilitated levels of reporter mRNA accumulation that were above that of the intronless cassette and comparable to the ADH1 cassette (see FIG. 6). These results show that i1, 12, i5, i6, and i7 (SEQ ID NOS: 52-53, 56-58 respectively) display intron-mediated enhancement of expression in this system.

### Materials and Methods

Introns i1 (SEQ ID NO: 52), i2 (SEQ ID NO: 53), i3 (SEQ ID NO: 54), i4 (SEQ ID NO: 55) and i5 (SEQ ID NO: 56) were generated using a method known in the art as oligonucleotide stacking. Oligos and primers (Table 4) synthesized by IDT

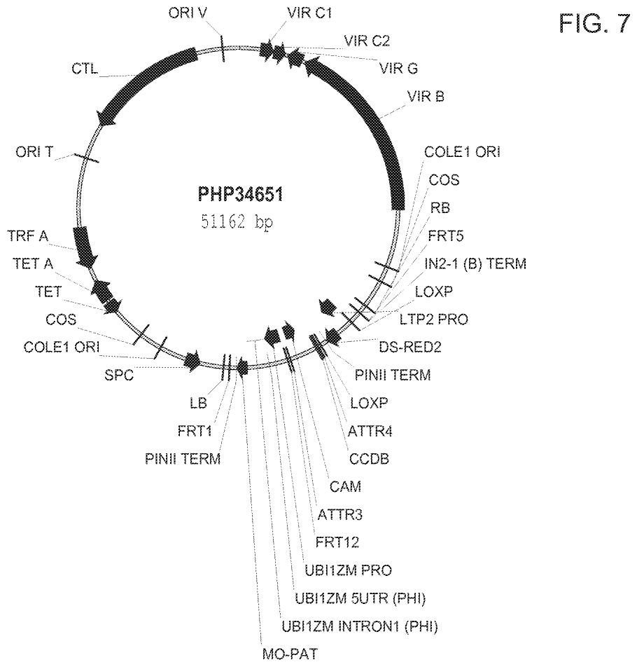

(Integrated DNA Technologies, Inc. Coralville, IA) were resuspended in distilled water to a concentration of 100 μM. Equal amounts of each oligonucleotide were mixed to create a total volume of 10 μl. The flanking primers for PCR amplification were also mixed equally to a volume of 10 μl. Two microliters of the oligonucleotide mix and 10 μl of the primer mix were combined for PCR using the HotStart Herculase system from Stratagene. PCR was performed using 10 μl Herculase buffer, 2 μl of 25 nM dNTPs, 1.2 μl of the oligo and primer mixture, 1 μl 100 mM MgSO4, 2 μl DMSO, 1 μl HotStart Herculase enzyme, and 82.8 μl of distilled water. PCR conditions were 96° C. for 3 minutes, then 35 cycles at 94° C. for 30 s, 60° C. for 30 s, and 72° C. for 1 min., followed by 72° C. for 10 min. Reactions were stored at 4° C. Introns i6 and i7 were synthesized by GENEART, Inc., Burlingame, CA. To clone introns i1 (SEQ ID NO: 52), i2 (SEQ ID NO: 53), i6 (SEQ ID NO: 57), and i7 (SEQ ID NO: 58), the starting product was cut with the restriction enzymes ECORV (5′ end) and BamHI (3′ end). Intron i5 (SEQ ID NO: 56), was cut with EcoRV (5′ end) and Bglll (3′ end). A plasmid containing a cassette (SEQ ID NO: 59, PHP38808; FIG. 5) with the CYMV promoter the ADH1 intron and an insecticidal gene flanked by GATEWAY® (INVITROGEN™) attL recombination sites was cut with EcoRV and BamHI to remove the ADH1 intron and allow the experimental introns to be ligated into the cut plasmid. The resulting vectors (entry vectors, PHP38811, PHP38813, PHP38815, PHP38817, PHP38819, PHP38821, PHP38823 for i1, i2, i3, i4, i5, i6, i7 respectively) were used in LR reactions with a larger plasmid (PHP34651, FIG. 7, SEQ ID NO: 60) containing GATEWAY® attR recombination sites and a PAT expression cassette to generate the final expression vectors (destination vectors PHP38812, PHP38814, PHP38816, PHP38818, PHP38820, PHP38822 and PHP38824 respectively for introns i1, i2, i3, i4, i5, i6, 17, 18 and i9). These vectors were used to transform competent Agrobacterium tumefaciens cells, which were then used to transiently transform BMS cells.

TABLE 4
Primers and Oligonucleotides Used for Oligonucleotide Stacking

| Oligo/Primer | (Used for) | Sense/ | Flanking |
|---|---|---|---|
| SEQ ID NO: | Intron | Antisense | Primer/Oligonucleotide |
| 61 | i1 | Sense | Flanking Primer |
| 62 | i1 | Sense | Oligonucleotide |
| 63 | i1 | Sense | Oligonucleotide |
| 64 | i1 | Sense | Oligonucleotide |
| 65 | i1 | Antisense | Oligonucleotide |
| 66 | i1 | Antisense | Oligonucleotide |
| 67 | i1 | Antisense | Oligonucleotide |
| 68 | i1 | Antisense | Flanking Primer |
| 69 | i2 | Sense | Flanking Primer |
| 70 | i2 | Sense | Oligonucleotide |
| 71 | i2 | Sense | Oligonucleotide |
| 72 | i2 | Sense | Oligonucleotide |
| 73 | i2 | Antisense | Oligonucleotide |
| 74 | i2 | Antisense | Oligonucleotide |
| 75 | i2 | Antisense | Oligonucleotide |
| 76 | i2 | Antisense | Flanking Primer |
| 77 | i3 | Sense | Flanking Primer |
| 78 | i3 | Sense | Oligonucleotide |
| 79 | i3 | Sense | Oligonucleotide |
| 80 | i3 | Antisense | Oligonucleotide |
| 81 | i3 | Antisense | Oligonucleotide |
| 82 | i3 | Antisense | Flanking Primer |
| 83 | i4 | Sense | Flanking Primer |
| 84 | i4 | Sense | Oligonucleotide |
| 85 | i4 | Sense | Oligonucleotide |
| 86 | i4 | Antisense | Oligonucleotide |
| 87 | i4 | Antisense | Oligonucleotide |
| 88 | i4 | Antisense | Flanking Primer |
| 89 | i5 | Sense | Flanking Primer |
| 90 | i5 | Sense | Oligonucleotide |
| 91 | i5 | Sense | Oligonucleotide |
| 92 | i5 | Antisense | Oligonucleotide |
| 93 | i5 | Antisense | Oligonucleotide |
| 94 | i5 | Antisense | Flanking Primer |

RNA was extracted from infiltrated tissue culture material using the QIAGEN® RNA Maxiprep kit. Based on ELISA data for PAT, RNA samples were loaded on an agarose gel (1% Lonza SeaKem LE agarose) to contain equal parts per million of PAT to normalize for variations in transformation efficiency. After electrophoresis, samples on the gel were transferred to a nylon membrane via capillary transfer overnight using the WHATMAN® TurboBlotter system standard protocol. RNA was crosslinked to the membrane by UV light. Prehybridization and hybridization steps were performed following the manufacturer's protocol for Roche DIG Easy Hyb solution (catalog #11603558001). The blot was prehybridized at 50° C. in Roche DIG Easy Hyb solution, then was probed overnight at 50° C. with a mixture of digoxigenin-labeled DNA probes for the insecticidal and PAT gene in Roche DIG Easy Hyb solution. Probes were generated using Roche PCR DIG Probe Synthesis Kit (Roche catalog #11636090910). The blot was washed twice for five minutes each at room temperature in low stringency buffer (2×SSC+0.1% SDS), then washed twice for 15 minutes each at 50° C. in high stringency buffer (0.1×SSC+0.1% SDS).

For detection, the Roche DIG Wash and Block Buffer Set (catalog #11585762001) was used. The membrane was washed for 2 minutes at room temperature in wash buffer, and then blocked in block solution for 30 minutes at room temperature. A 1:10,000 dilution of anti-digoxigenin-AP antibody (Roche catalog #11093274910, 0.75 U/μl) in 50 ml block solution was added to the blot for 30 minutes. The blot was washed twice for 15 minutes each at room temperature in wash buffer, and then equilibrated in 50 ml of detection buffer for 3 minutes. Blot was incubated at room temperature for 5 minutes with 3 ml of CSPD (Roche catalog #1755633001), and then incubated at 37° C. for 10 minutes. Detection was done with film at 37° C.

### Example 8

Identification of Unique Motif from Maize First Introns Using the Experimental Dataset of Tested Enhancing Introns

Computational analysis was performed to identify unique motifs that were present in the 9 enhancing introns identified as explained in Examples 4 and 7 and Table 1 (TS1, TS7, TS13, TS27, 11, 12, i5, 16, 17 (SEQ ID NOS: 4, 8, 13, 19, 52, 53, 56, 57, and 58 respectively)). The proprietary promoter REAPer tool was adapted to look for possibly conserved motifs. The promoter REAPer tool is a regulatory element identification tool that relies on the conserved word approach. It is described in the U.S. patent application Ser. No. 12/534,471. The introns were searched in both directions using sets of 3-6 introns at a time. When candidates were found, they were used to search all the introns.

The introns were divided into the following categories. “All Enhancing Introns” are the 9 introns (new enhancing introns) described in Table 1 and experimentally shown to be enhancing gene expression (TS1, TS7, TS13, TS27, i1, 12, i5, i6, and i7 (SEQ ID NOS: 4, 8, 13, 19, 52, 53, 56, 57, and 58 respectively), plus four known enhancing introns (Adh1_intron1 (SEQ ID NO: 95), Adh1_intron 6 (SEQ ID NO: 96), Sh-1_intron 1 (SEQ ID NO: 97) and Ubi1ZM_intron (SEQ ID NO: 98) Callis, J. et al (1987) Genes Dev. 1:1183-1200, Vasil, V. et al (1989) Plant Physiol. 91; 1575-1579, Christensen, A. H. et al (1992) Plant Mol. Biol. 18:675-689, Jeong, Y.-M. et al (2009) Plant Sci. 176:58-65). The 10 “non-enhancing introns” are 10 introns found not to enhance gene expression in transient maize assays as explained in Examples 4 and 7 and Table 1 (SEQ ID NOS: 5-7, 9, 11, 12, 17, 18, 54, and 55).

The 8-bp sequence CAGATCTG (SEQ ID NO: 99) or its variations were found in all the enhancing introns except TS27. The exact 8-bp sequence CAGATCTG was found in 2 out of the 9 enhancing introns identified (SEQ ID NOS: 52 and 53), but was not found in any of the 10 non-enhancing introns (SEQ ID NOS: 5-79, 11, 12, 17, 18, 54, and 55). A subset of this sequence ATCTG (SEQ ID NO: 100) was also present in 8 out of 9 enhancing introns (SEQ ID NOS: 4, 8, 13, 52, 53, 56, 57 and 58), and was also found to be present in the four known enhancing introns (SEQ ID NOS: 95-98). The frequency of occurrence of these motifs was normalized to the intron length (Table 6).

The variations of the 8-bp sequence CAGATCTG are mainly in the first 3 base pairs. The motif variations can be represented as the consensus sequence, Y[R/T]RATCYG (SEQ ID NO: 146). The first position can be any of the two pyrimidine bases, C or T. The second position can be substituted by an A, G or T and the third position can any purine. The last 5 base pairs of the sequence, that is the sequence ATCTG is highly conserved.

### Statistical Analyses of Motif Frequencies:

A number of simple frequency statistics were determined for the introns. The statistics are shown in Tables 5 and 6.

TABLE 5

| | | Aggregate | Average Intron |
|---|---|---|---|
| Intron Classification | Intron Count | Nts | Length |
| All Enhancing Introns | 13 | 7716 | 594 |
| New Enhancing Introns | 9 | 4813 | 535 |
| Other Enhancing Introns | 4 | 2903 | 726 |
| Non-Enhancing Introns | 10 | 7888 | 789 |
| Non-Tested Introns | 1066 | 933097 | 875 |

TABLE 6

| | | | Frequency | Frequency |
|---|---|---|---|---|
| | Total Introns | Total Introns | Intron | Intron |
| | Containing | Containing | Contains | Contains |
| Intron Classification | CAGATCTG | ATCTG | CAGATCTG | ATCTG |
| All Enhancing Introns | 2 | 12 | 0.15 | 0.92 |
| New Enhancing | 2 | 8 | 0.22 | 0.89 |
| Introns | | | | |
| Other Enhancing | 0 | 4 | 0.00 | 1.00 |
| Introns | | | | |
| Non-Enhancing | 0 | 7 | 0.00 | 0.70 |
| Introns | | | | |
| Non-Tested Introns | 15 | 502 | 0.01 | 0.47 |
| Ratio All | | 1.71 | | 1.32 |
| Enhancing/Non- | | | | |
| Enhancing | | | | |
| Ratio New | | 1.14 | | 1.27 |
| Enhancing/Non- | | | | |
| Enhancing | | | | |
| | Total | Total | | |
| | Occurrences | Occurrences | Gross | Gross |
| | CAGATCTG | ATCTG | Frequency | Frequency |
| Intron Classification | Either Strand | Either Strand | CAGATCTG | ATCTG |
| All Enhancing Introns | 6 | 29 | 0.0008 | 0.0038 |
| New Enhancing | 6 | 23 | 0.0012 | 0.0048 |
| Introns | | | | |
| Other Enhancing | 0 | 6 | 0 | 0.00207 |
| Introns | | | | |
| Non-Enhancing | 0 | 18 | 0 | 0.00228 |
| Introns | | | | |
| Non-Tested Introns | 15 | 1391 | 1.6075E-05 | 0.00149 |
| Ratio All | | 1.61 | | 1.647 |
| Enhancing/Non- | | | | |
| Enhancing | | | | |
| Ratio New | | 1.28 | | 2.094 |
| Enhancing/Non- | | | | |
| Enhancing | | | | |
| | Average | Average of | | |
| | Individual | Individual | SE | |
| | Frequency of | Intron | Frequency | SE |
| | CAGATCTG/ | Frequency of | CAGATCTG/ | Frequency |
| Intron Classification | kb | ATCTG/kb | kb | ATCTG/kb |
| All Enhancing Introns | 0.0036 | 0.0094 | 0.0025 | 0.0004 |
| New Enhancing | 0.0052 | 0.0124 | 0.0035 | 0.0050 |
| Introns | | | | |
| Other Enhancing | 0.00000 | 0.00266 | 0.00000 | 0.00107 |
| Introns | | | | |
| Non-Enhancing | 0.00000 | 0.00203 | 0.00000 | 0.00057 |
| Introns | | | | |
| Non-Tested Introns | 0.00013 | 0.00271 | 0.00005 | 0.00013 |
| Ratio All | | 4.62 | | |
| Enhancing/Non- | | | | |
| Enhancing | | | | |
| Ratio New | | 6.10 | | |
| Enhancing/Non- | | | | |
| Enhancing | | | | |

SE frequency is standard error of frequency. Gross frequency is simply the total occurrences divided by the aggregate nucleotides of all the introns in the set.

The ‘all’ 13 enhancing introns have 4.6-fold higher, and the 9 ‘new’ enhancing introns have 6.1-fold higher frequencies of ATCTG relative to the non-enhancing introns on a mean frequency per kb of intron basis (See Tables 5 and 6 above).

### Example 9

Identification of Novel Maize Introns with 8-bp Motif

From the initial set of 1085 introns explained in Example 1, 1066 introns that were still not tested experimentally were scanned computationally to identify the ones with the 8-bp motif. Four introns (SEQ ID NOS: 101-104) were found to contain the exact 8-bp motif and these are good candidates for being enhancing introns.

### Example 10

### Identifying Promoters of Expression-Enhancing Introns

It is likely that the expression enhancing introns from Examples 4, 7 and 9 perform optimally along with their endogenous promoters. To test this 1000 bp-2000 bp of promoter regions upstream of the start codon from the respective genes (SEQ ID NOS: 105-117, SEQ ID NOS: 136 and 139) were identified and these can be tested with the respective introns.

### Cloning Endogenous Promoters of Expression Enhancing Introns

We amplified 1000 base pairs region of endogenous promoter, (using the primers given in Table 7) upstream of the start codon of the gene that carries TS1 intron as its first intron and cloned the pTS1v sequence (SEQ ID NO: 136) in ITVUR-2 vector (SEQ ID NO: 3, PHP41353) between Ascl-AsiS1 restriction sites, followed by the TS1 intron (SEQ ID NO: 4) at AsiSI-Acc65I sites to create an endogenous promoter and intron combination (PHP50061). Similarly, we amplified a 1487 base pair region of endogenous promoter (pTS27v; SEQ ID NO: 139) upstream of the TS27 intron and cloned it in ITVUR-2 vector (SEQ ID NO: 3, PHP41353) at Ascl-AsiS1 restriction sites, followed by the TS27v intron (SEQ ID NO: 138) at AsiSI-Acc651 sites to give us an endogenous promoter and intron combination (PHP52322).

### Example 11

### Cloning and Testing of TS2 Enhancing Intron and Corresponding Endogenous Promoter

We tested another intron with potential gene expression enhancing properties. TS2 intron (SEQ ID NO: 118) was cloned into ITVUR-2 vector (SEQ ID NO: 3, PHP41353) using the same procedure as explained in Example 3 to create PHP50062. We created 2 more constructs to test the ability of the endogenous promoter upstream of the start codon of the gene that carries TS2 as its first intron to drive gene expression and ability of TS2 intron to enhance gene expression. We amplified 1077-bp of endogenous TS2 promoter (pTS2; SEQ ID NO: 119), as defined by the sequence upstream of the TS2 intron at the genomic location, and cloned that in ITVUR-2 vector (SEQ ID NO: 3) between Ascl and Ncol sites (PHP500063). We also amplified the pTS2 promoter and TS2 intron sequence from the endogenous locus (1077 bp promoter (SEQ ID NO: 118)+1329 bp intron (SEQ ID NO: 119)) and cloned that between Ascl and Ncol sites (PHP50111). The primers for these amplifying promoter and intron sequences to make these constructs are given in Table 2 and Table 7.

TABLE 7

| Cloned sequence | | Forward Primer | Reverse Primer |
|---|---|---|---|
| Promoter | Intron | (SEQ ID NO) | (SEQ ID NO) |
| — | TS2 (SEQ ID | 120 | 121 |
| | NO: 118) | | |
| pTS2 (SEQ ID | TS2 (SEQ ID | 122 | 123 |
| NO: 119) | NO: 118) | | |
| pTS2 (SEQ ID | — | 122 | 124 |
| NO: 119) | | | |
| pTS1v (SEQ ID | — | 125 | 126 |
| NO: 136) | | | |
| pTS27v (SEQ | — | 127 | 128 |
| ID NO: 139) | | | |

All the constructs were mobilized into the Agrobacterium strain LBA4404/pSB1 and selected on spectinomycin and tetracycline. Agrobacterium transformants were isolated and the integrity of the plasmid was confirmed by retransforming to E. coli or PCR analysis.

### Example 12

Stable Transfection of Rice with Promoter and Intron Sequence Constructs

### Transformation and Regeneration of Rice Callus Via Agrobacterium Infection

O. sativa spp. japonica rice var. Nipponbare seeds are sterilized in absolute ethanol for 10 minutes then washed 3 times with water and incubated in 70% Sodium hypochlorite [Fisher Scientific-27908] for 30 minutes. The seeds are then washed 5 times with water and dried completely. The dried seeds are inoculated into NB-CL media [CHU (N6) basal salts (PhytoTechnology-C416) 4 g/l; Eriksson's vitamin solution (1000× PhytoTechnology-E330) 1 ml/l; Thiamine HCl (Sigma-T4625) 0.5 mg/l; 2,4-Dichloro phenoxyacetic acid (Sigma-D7299) 2.5 mg/l; BAP (Sigma-B3408) 0.1 mg/l; L-Proline (PhytoTechnology-P698) 2.5 g/l; Casein acid hydrolysate vitamin free (Sigma-C7970) 0.3 g/l; Myo-inositol (Sigma-13011) 0.1 g/l; Sucrose (Sigma-S5390) 30 g/l; GELRITE® (Sigma-G1101.5000) 3 g/l; pH 5.8) and kept at 28° C. in dark for callus proliferation.

A single Agrobacterium colony containing a desired insert with the candidate sequences from a freshly streaked plate can be inoculated in YEB liquid media [Yeast extract (BD Difco-212750) 1 g/l; Peptone (BD Difco-211677) 5 g/l; Beef extract (Amresco-0114) 5 g/l; Sucrose (Sigma-S5390) 5 g/l; Magnesium Sulfate (Sigma-M8150) 0.3 g/l at pH-7.0] supplemented with Tetracycline (Sigma-T3383) 5 mg/l, Rifamysin 10 mg/l and Spectinomycin (Sigma-5650) 50 mg/l. The cultures are grown overnight at 28° C. in dark with continuous shaking at 220 rpm. The following day the cultures are adjusted to 0.5 Absorbance at 550 nm in PHI-A (CHU (N6) basal salts (PhytoTechnology-C416) 4 g/l; Eriksson's vitamin solution (1000× PhytoTechnology-E330) 1 ml/l; Thiamine HCl (Sigma-T4625) 0.5 mg/l; 2,4-Dichloro phenoxyacetic acid (Sigma-D7299) 2.5 mg/l, L-Proline (PhytoTechnology-P698) 0.69 mg/l; Sucrose (Sigma-S5390) 68.5 g/l; Glucose-36 g/l (Sigma-G8270); pH 5.8);) media supplemented with 200 μM Acetosyringone (Sigma-D134406) and incubated for 1 hour at 28° C. with continuous shaking at 220 rpm.

17-21 day old proliferating calli are transferred to a sterile culture flask and Agrobacterium solution prepared as described above was added to the flask. The suspension is incubated for 20 minutes with gentle shaking every 2 minutes. The Agrobacterium suspension is decanted carefully and the calli are placed on WHATMAN filter paper No—4. The calli are immediately transferred to NB-CC medium [NB-CL supplemented with 200 μM Acetosyringone (Sigma-D134406) and incubated at 21° C. for 72 hrs.

### Culture Termination and Selection

The co-cultivated Calli are placed in a dry, sterile, culture flask and washed with 1 liter of sterile distilled water containing Cefotaxime (Duchefa-C0111.0025) 0.250 g/l and Carbenicillin (Sigma-C0109.0025) 0.4 g/l. The washes are repeated 4 times or until the solution appeared clear. The water is decanted carefully and the calli are placed on WHATMAN filter paper No—4 and dried for 30 minutes at room temperature. The dried calli are transferred to NB-RS medium [NB-CL supplemented with Cefotaxime (Duchefa-C0111.0025) 0.25 g/l; and Carbenicillin (Sigma-C0109.0025) 0.4 g/l and incubated at 28° C. for 4 days.

The calli are then transferred to NB-SB media [NB-RS supplemented with Bialaphos (Meiji Seika K.K., Tokyo, Japan) 5 mg/l and incubated at 28° C. and subcultured into fresh medium every 14 days. After 40-45 days on selection, proliferating, Bialaphos resistant, callus events are easily observable.

Regeneration of Stably Transformed Rice Plants from Transformed Rice Calli

Transformed callus events are transferred to NB-RG media [CHU (N6) basal salts (PhytoTechnology-C416) 4 g/l; N6 vitamins 1000×1 ml {Glycine (Sigma-47126) 2 g/l; Thiamine HCl (Sigma-T4625) 1 g/l; acid; Kinetin (Sigma-K0753) 0.5 mg/l; Casein acid hydrolysate vitamin free (Sigma-C7970) 0.5 g/l; Sucrose (Sigma-S5390) 20 g/l; Sorbitol (Sigma-S1876) 30 g/l, pH was adjusted to 5.8 and 4 g/l GELRITE® (Sigma-G1101.5000) was added. Post-sterilization 0.1 ml/l of CuSo4 (100 mM concentration, Sigma-C8027) and 100 ml/l 10×AA Amino acids pH free {Glycine (Sigma-G7126) 75 mg/l; L-Aspartic acid (Sigma-A9256) 2.66 g/l; L-Arginine (Sigma-A5006) 1.74 g/l; L-Glutamine (Sigma-G3126) 8.76 g/l} and incubated at 32° C. in light. After 15-20 days, regenerating plantlets can be transferred to magenta boxes or tubes containing NB-RT media [MS basal salts (PhytoTechnology-M524) 4.33 g/L; B5 vitamins 1 ml/l from 1000× stock {Nicotinic acid (Sigma-G7126) 1 g/l, Thiamine HCl (Sigma-T4625) 10 g/l)}; Myo-inositol (Sigma-13011) 0.1 g/l; Sucrose (Sigma-S5390) 30 g/l; and IBA (Sigma-15386) 0.2 mg/l; pH adjusted to 5.8]. Rooted plants obtained after 10-15 days can be hardened in liquid Y media [1.25 ml each of stocks A-F and water sufficient to make 1000 ml. Composition of individual stock solutions: Stock (A) Ammonium Nitrate (HIMEDIA-RM5657) 9.14 g/l, (B) Sodium hydrogen Phosphate (HIMEDIA-58282) 4.03 g, (C) Potassium Sulphate (HIMEDIA-29658-4B) 7.14 g, (D) Calcium Chloride (HIMEDIA-C5080) 8.86 g, (E) Magnesium Sulphate (HIMEDIA-RM683) 3.24 g, (F) (Trace elements) Magnesium chloride tetra hydrate (HIMEDIA-10149) 15 mg, Ammonium Molybdate (HIMEDIA-271974B) 6.74 mg/l, Boric acid (Sigma-136768) 9.34 g/l, Zinc sulphate heptahydrate (HiMedia-RM695) 0.35 mg/l, Copper Sulphate heptahydrate (HIMEDIA-C8027) 0.31 mg/l, Ferric chloride hexahydrate (Sigma-236489) 0.77 mg/l, Citric acid monohydrate (HIMEDIA-C4540) 0.119 g/l] at 28° C. for 10-15 days before transferring to greenhouse. Leaf samples are collected for histochemical GUS staining with 5-bromo-4-chloro-3-indolyl-B-D-glucuronide (X-Gluc), using standard protocols (Janssen and Gardner, Plant Mol. Biol. (1989) 14:61-72).

Transgenic plants are analyzed for copy number by southern blotting using standard procedure. All single copy events are transferred to individual pots and further analysis is performed only on these. For all the analysis leaf material from three independent one month old single copy T₀ events were taken.

### Transgene Copy Number Determination by Quantitative PCR

Transgenic rice plants generated using different constructs were analyzed to determine the transgene copy number using TaqMan-based quantitative real-time PCR (qPCR) analysis. Genomic DNA was isolated from the leaf tissues collected from 10-day old TO rice plants using the QIAGEN® DNEASY® Plant Maxi Kit (QIAGEN® Inc.) according to the manufacturer's instructions. DNA concentration was adjusted to100 ng/μl and was used as a template for the qPCR reaction to determine the copy number. The copy number analysis was carried out by designing PCR primers and TaqMan probes for the target gene and for the endogenous glutathione reductase 5 (GR5) gene. The endogenous GR5 gene serves as an internal control to normalize the Ct values obtained for the target gene across different samples. In order to determine the relative quantification (RQ) values for the target gene, genomic DNA from known single and two copy calibrators for a given gene were also included in the experiment. Test samples and calibrators were replicated twice for accuracy. Non-transgenic control and no template control were also included in the reaction. The reaction mixture (for a 20 μl reaction volume) comprises 10 μl of 2× TaqMan universal PCR master mix (Applied Biosystems), 0.5 μl of 10 μM PCR primers and 0.5 μl of 10 μM TaqMan probe for both target gene and endogenous gene. Volume was adjusted to 19 μl using sterile Milli Q water and the reaction components were mixed properly and spun down quickly to bring the liquid to bottom of the tube. 19 μl of the reaction mix was added into each well of reaction plate containing 1 μl of genomic DNA to achieve a final volume of 20 μl. The plate was sealed properly using MicroAmp optical adhesive tape (Applied Biosystems) and centrifuged briefly before loading onto the Real time PCR system (7500 Real PCR system, Applied Biosystems). The amplification program used was: 1 cycle each of 50° C. for 2:00 min and 95° C. for 10:00 min followed by 40 repetitions of 95° C. for 15 sec and 58° C. for 1:00 min. After completion of the PCR reaction, the SDS v2.1 software (Applied Biosystems) was used to calculate the RQ values in the test samples with reference to single copy calibrator.

Stable transgenic rice events were generated with the constructs, PHP50063, PHP50111 PHP50062, PHP50061, PHP52322, and PHP42365 as given in Table 8. The primers used for amplifying the cloned promoter and intron sequences for these constructs are given in Table 2 and Table 7.

TABLE 8
Description of Promoter and Intron Elements in Constructs

| Construct | Intron | Promoter |
|---|---|---|
| PHP50063 | — | pTS2 |
| | | (SEQ ID NO: 119) |
| PHP50111 | TS2 | pTS2 |
| | (SEQ ID NO: 118) | (SEQ ID NO: 119) |
| PHP50062 | TS2 | Zm Ubi promoter |
| | (SEQ ID NO: 118) | |
| PHP50061 | TS1 | pTS1v |
| | (SEQ ID NO: 4) | (SEQ ID NO: 136) |
| PHP52322 | TS27v | pTS27v |
| | (SEQ ID NO: 138) | (SEQ ID NO: 139) |
| PHP42365 | Zm Ubi intron | Zm Ubi promoter |

The stable transgenic rice events generated with these constructs were subjected to TaqMan-based qPCR (quantitative PCR) analysis to determine the transgene copy number as described above. PCR primers and TaqMan probes designed for the GUS reporter gene and for the endogenous GR5 gene are listed in Table 9.

TABLE 9
Primer Sequences for qPCR

| Primer ID | SEQ ID NO: |
|---|---|
| GUS F primer | 129 |
| GUS R primer | 130 |
| GR5, F primer | 131 |
| GR5, R primer | 132 |

TABLE 10
Probe Sequences for qPCR

| | SEQ ID NO | Probe | Quencher |
|---|---|---|---|
| GUS | 133 | Fam | Tamra |
| GR5 | 134 | Vic | MGB |

All single copy events were transferred to individual pots and further analysis was performed on leaf material and panicle collected one month after transplanting in the greenhouse.

### Qualitative and Quantitative Analysis of GUS Reporter Gene Expression in Stable Rice Events

Both qualitative and quantitative GUS reporter gene expression analyses were carried out in triplicates on at least 5 independent single copy events for each construct. Leaf and panicle samples were collected for histochemical GUS staining with 5-bromo-4-chloro-3-indolyl-B-D-glucuronide (X-Gluc), using standard protocols (Janssen and Gardner, Plant Mol. Biol. (1989) 14:61-72) and for quantitative MUG assay using standard protocols (Jefferson, R. A., Nature. 342, 837-8 (1989); Jefferson, R. A., Kavanagh, T. A. & Bevan, M. W., EMBO J. 6, 3901-3907 (1987).

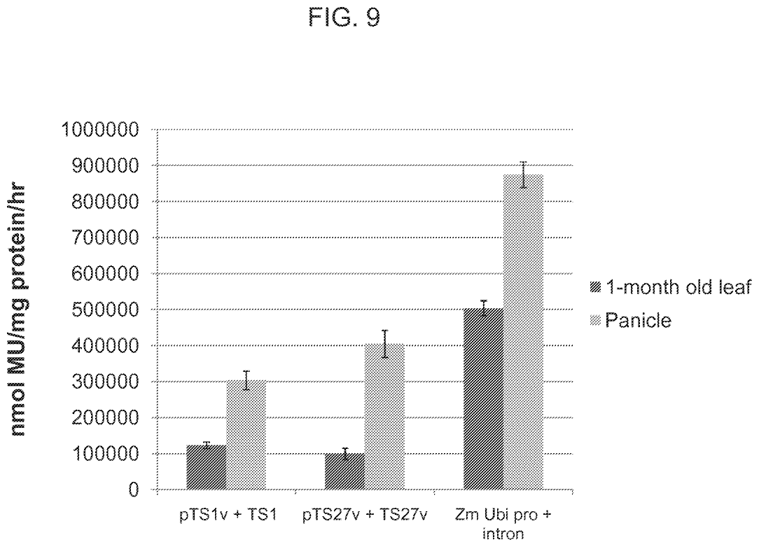

TS1 and TS27v when combined with their respective endogenous promoters (pTS1v+TS1 (PHP50061) and pTS27v+TS27v (PHP52322) were able to drive GUS expression in stable rice transgenic events (FIG. 9).

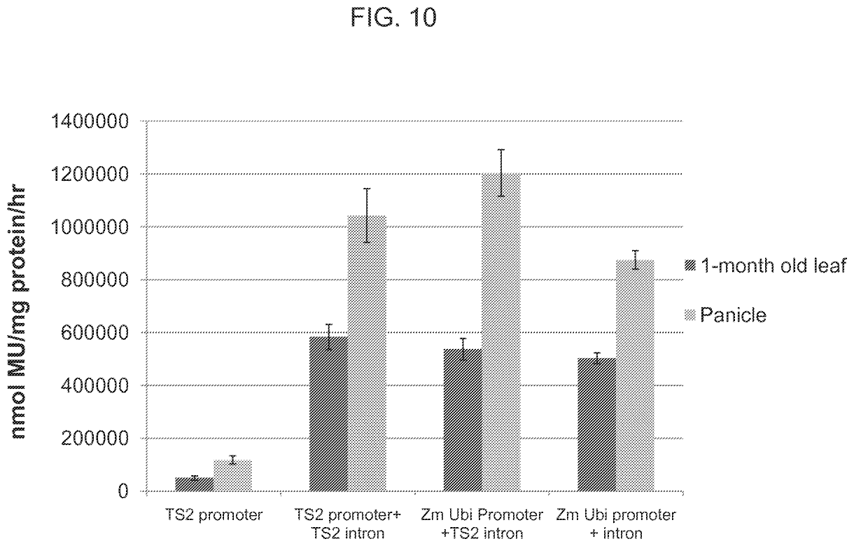

TS2 intron with its endogenous promoter (PHP50111) enhanced the GUS reporter gene expression by 11.6 fold in leaves and 8.9 fold in panicles compared to the TS2 promoter alone (PHP50063) driving the GUS reporter gene expression (FIG. 10) and the values obtained were comparable to the levels observed with maize ubiquitin promoter and intron (PHP42365) driving GUS in transgenic rice plants. There is a slight increase in the GUS reporter gene expression levels when the TS2 intron is cloned with maize Ubiquitin promoter (PHP50062) compared to the data obtained with maize ubiquitin intron cloned with maize ubiquitin promoter (FIG. 10).

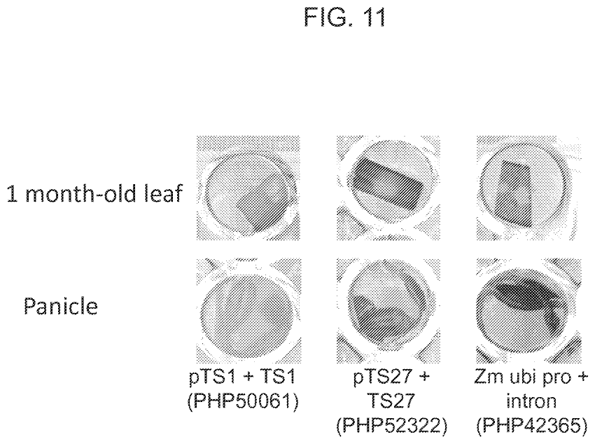

GUS histochemical staining data were found to correlate very well with the quantitative GUS assay in all events. Representative images are shown in FIG. 10 and FIG. 11.

### Example 13

### Identification of Novel Terminator Sequences

Transcription terminators for the 4 genes comprising the expression enhancing introns TS1, TS2, TS13 and TS27v (SEQ ID NOS: 4, 118, 13 and 138 respectively) were identified, and were called tTS1 (SEQ ID NO: 140), tTS2 (SEQ ID NO: 141), tTS13 (SEQ ID NO: 142) and tTS27 (SEQ ID NO: 143). Terminator sequences were defined as 500-900 bp of sequence downstream of the translational stop codon of the respective genes.

### Example 14

### Amplification and Cloning of Terminator Sequences

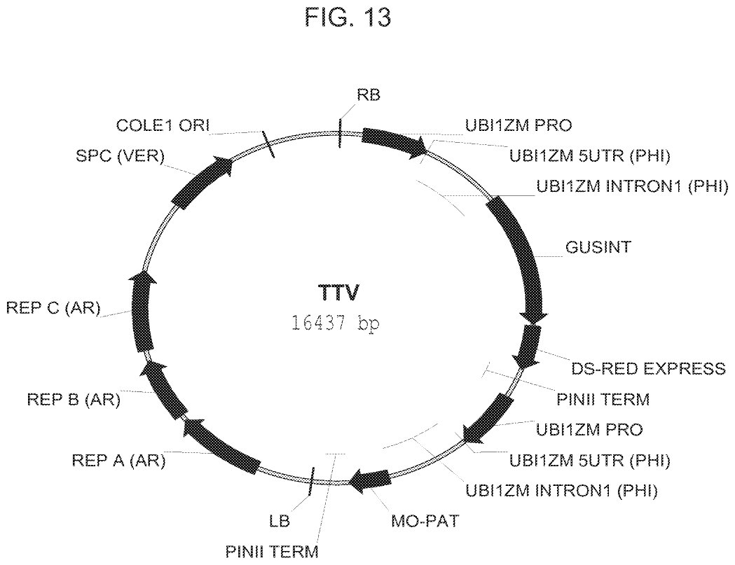

We constructed a terminator test vector (TTV) (PHP49597—FIG. 13; SEQ ID NO: 144) carrying GUS (B-glucuronidase) reporter gene driven by the Maize Ubiquitin promoter using standard molecular biology techniques (Sambrook et al.). A promoterless Ds-RED coding sequence was included downstream of the GUS gene for measurement of transcription downstream of the cloned test terminator sequences (read-through transcripts). The Ds-Red sequence was followed by a Pinll terminator to enable termination and polyadenlylation of all transcripts, so we could detect them by reverse-transcription-PCR (RT-PCR) using oligo dT primer. The Terminator test vector also carried a monocot-optimized Phosphinothricin acetyl transferase (MOPAT) gene as a plant selectable marker.

Candidate terminator sequences can be amplified from maize genomic DNA. The resulting DNA fragments can be cloned into the terminator test vector at Acc651 restriction site using IN-FUSION™ cloning (Clontech Inc.). All constructs will be transformed into Agrobacterium (LBA4404/pSB1)

### Example 15

Rice Transformation with Candidate Terminator Sequences

The candidate maize terminator sequences tTS1, tTS2, tTS13 and tTS27 (SEQ ID NOS: 140-143 respectively) will be tested for their ability to function as transcription terminators in stable rice transgenic plants generated by Agrobacterium mediated transformation as described in Example 12.

### Example 16

### Testing of Candidate Rice Terminator Sequences in Stably Transformed Rice Tissues

Reverse Transcriptase-PCR (RT-PCR) and GUS assays can be done from stably transformed rice plant tissues, to test the ability of candidate maize terminator sequences tTS1, tTS2, tTS13 and tTS27 (SEQ ID NOS: 140-143 respectively) to prevent transcription read-through and to compare GUS expression

### Reverse Transcription PCR (RT-PCR) to Determine Transcription Read-Through

RNA will be extracted from leaf tissue from multiple independent TO events for each construct. cDNA can be synthesized using SuperScript® III First-Strand Synthesis System from Invitrogen. The level of GUS gene and read-through transcripts will be assayed using specific primers within GUS gene and DS-Red respectively. Transcript levels can also be measured by quantitative RT-PCR using primers and probes within GUS and DS-Red sequences.

### Histochemical and Fluorometric GUS Analysis

Tissue samples from each independent stably transformed rice line can be stained for histochemical GUS analysis, with 5-bromo-4-chloro-3-indolyl-β-D-glucuronide (X-Gluc), using standard protocols (Janssen and Gardner, Plant Mol. Biol. (1989) 14:61-72,). Tissue samples can also be used for quantitative MUG assay using standard protocols [Jefferson, R. A., Nature. 342:837-838 (1989); Jefferson, R. A., Kavanagh, T. A. & Bevan, M. W. EMBO J. 6:3901-3907 (1987)].

1. A recombinant DNA construct comprising an intron operably linked to a promoter, a heterologous polynucleotide, and a terminator wherein the intron comprises a nucleotide sequence that has at least 95% sequence identity to SEQ ID NO: 4, 8, 13, 19, 52, 53, 56, 57, 58, 101, 102, 103, 104, 118, 137 or 138.

2. (canceled)

3. (canceled)

4. The recombinant DNA construct of claim 1, wherein
the promoter comprises a nucleotide sequence that has at least 95% identity to SEQ ID NO: 105, 106, 107, 108, 109, 110, 111, 112, 113, 114, 115, 116, 117, 119, 136 or 139.

5. The recombinant DNA construct of claim 4, wherein
the terminator comprises a nucleotide sequence that has at least 95% identity to SEQ ID NO: 140, 141, 142 or 143.

6. The recombinant DNA construct of claim 1, wherein the intron comprises the nucleotide sequence of SEQ ID NO: 4, 8, 13, 19, 52, 53, 56, 57, 58, 101, 102, 103, 104, 118, 137 or 138.

7. The recombinant DNA construct of claim 4, wherein the promoter comprises the nucleotide sequence of SEQ ID NO: 105, 106, 107, 108, 109, 110, 111, 112, 113, 114, 115, 116, 117, 119, 136 or 139.

8. The recombinant DNA construct of claim 5, wherein the terminator comprises the nucleotide sequence of SEQ ID NO: 140, 141, 142 or 143.

9. (canceled)

10. The recombinant DNA construct of claim 1, wherein the intron enhances expression of the heterologous polynucleotide in a plant.

11. (canceled)

12. (canceled)

13. (canceled)

14. (canceled)

15. (canceled)

16. A plant comprising the recombinant DNA construct of claim 10.

17. A seed comprising the recombinant DNA construct of claim 10.

18. (canceled)

19. (canceled)

20. (canceled)

21. (canceled)

22. (canceled)

23. A method for identifying an intron useful for enhancing transgene expression in a monocotyledenous plant comprising the steps of:
(a) scanning a plurality of monocot introns for the presence of a sequence motif identical to SEQ ID NO: 99;
(b) selecting an intron from step (a) comprising a nucleotide sequence that contains at least one copy of a sequence motif identical to SEQ ID NO: 99; and
(c) measuring the enhancing effect of the intron of step (b) on the expression of an operably linked heterologous polynucleotide.

24. (canceled)

25. A method for modulating transgene expression in a plant comprising the steps of:
(a) introducing into a regenerable plant cell the recombinant DNA construct of claim 1;
(b) regenerating a transgenic plant from the regenerable plant cell after step (a) wherein the transgenic plant comprises the recombinant DNA construct; and
(c) obtaining a progeny plant derived from the transgenic plant of step (b), wherein the progeny plant comprises the recombinant DNA construct and exhibits enhanced transgene expression when compared to a plant comprising in its genome the recombinant DNA construct without the corresponding intron sequence.

26. The method of claim 25 wherein said plant is a monocot.


SEQUENCE LISTING (146 sequences)

SEQ ID NO: 1 - DNA, 978 bp, Zea mays
  source 1..978: mol_type = genomic DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaag

SEQ ID NO: 2 - DNA, 1013 bp, Zea mays
  source 1..1013: mol_type = genomic DNA
gtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 3 - DNA, 16656 bp, synthetic construct
  misc_feature 1..16656: note = Vector Sequence
  source 1..16656: mol_type = other DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttaccagagctggtcacctttgtccaccaagatggaactgcggcctcgaagcttggcgcgccgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaagcgatcgcaggtacccgactttaacttagcctaggatccacacgacaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggtgacgtggggcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgcgatctgagcttctagaggatccccatcgatgggccccggccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcaggcctccaattcgtcaacttcgtccacagacatcaacatcttatcgtcctttgaagataagataataatgttgaagataagagtgggagcccccactaaaacattgctttgtcaaaagctaaaaaagatgatgcccgacagccacttgtgtgaagcatgagaagccggtccctccactaagaaaattagtgaagcatcttccagtggtccctccactcacagctcaatcagtgagcaacaggacgaaggaaatgacgtaagccatgacgtctaatcccaacttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgagaagccggtccctccactaagaaaattagtgaagcatcttccagtggtccctccactcacagctcaatcagtgagcaacaggacgaaggaaatgacgtaagccatgacgtctaatcccacaagaatttccttatataaggaacacaaatcagaaggaagagatcaatcgaaatcaaaatcggaatcgaaatcaaaatcggaatcgaaatctctcatctaacgtacgaccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaatgaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgggcccaggtagaatccgcctgagtcgcaagggtgacttcgcctatattggacgacggcgcgcagagggcgacctctttttgggttacgattgtaggattatcactaaaacaatacatgaacatattcaaatggcaatctctctaaggcattggaaataaatacaaataacagttgggtggagtttttcgacctgagggcgttaaccttctgttaacctaaaagctcttgcccaaacagcagaatcggcgctaattgccagcggcggaacttttccagtttcgcgaaaaatatcgccactggcaaggaatgggtttgagatggcgaagtctgtcctaaaagcagcgcctgtagttgtagggttgacggccttgatggagcgtcatgccgatgccctctcgagccaacttcaagcacatcatcttaaggttttcccgccgcattccgagaagggcattcgaacattcgggccatcggaggcgtccaagctgctcggcgttggcgagtcatatttacggcagaccgcgtctgagatgccagagttgaatgttagcatgagcccgggtggcaggcgaatgttctcaattgaagatatccatgtgattcggaagtatatggatcaggtcggccgcgggaaccggcgctacctgccacatcgtcgaggcggcgagcagcttcaggttatctctgtgatgaatttcaaaggtgggtcgggtaagaccaccaccgccgcgcatctggcgcagtacctcgctatgcgcggatatcgagtcttggccattgatctcgatcctcaagcgagcctttctgcactctttgggagccaaccggagacggacgttggcccgaacgaaacgctctacggcgctataaggtatgatgatgagcaggtggcaatcgaacgagtcgtccgagggacttacattcccgacctccacctgattcctggtaaccttgagctgatggagtttgaacacgatacgccacgcgcgctgatgaaccgcaaagagggcgacacgctcttttatggtcgcatcagccaagtaattgaagatatcgcggataactatgacgtcgtggtcatcgactgccctccccagcttgggtatctcacgctatccgcattgactgcggcgacgtccattcttgtcacggtccatccgcagatgctggatgtgatgtcgatgaaccagtttctggcaatgacatcgaaccttttgcgtgaaatcgagaatgctggcgccaagttcaagtttaattggatgcgctatctgataacccgtttcgaaccgagcgacggaccacagaaccaaatggtaggttatctgcggtcgatttttggcgaaaatgtcctcaattttccgatgcttaaaaccaccgcggtttcggacgctggcctgacaaaccagactctattcgaagtggagcgtggcctgttcacgcgctcgacctatgatcgagccttggaggcgatgaacgccgtcaacgacgagatcgaaacactgatcaaaaaagcatggggtaggcccacatgagccggaagcacatccttggcgtctcaactgacgcccctgagacgtcgcccgccgacaataggacggcaaagaaccgctccatgccgctcctcggcgtaacaaggaaggagcgcgatccggcaacgaagctcacagcgaacattggtaacgcactgcgagagcaaaacgatcgtcttagccgtgccgaagagatcgagcggcgtctcgctgaaggtcaggcagtgatagagttggatgcctcgtcaatagaaccgtctttcgtgcaggatcgtatgcgaggggacattgacgggctccttacttcgatccgggaacaaggacagcaagtcccaatccttgtgcgaccgcatccgagccagccgggccgatatcaggttgccttcggccaccgccggctacgcgccgtttcagaactcggacttccggtcagagcggtcgttcgcgaactgacggacgagcaagtggtcgtagcacagggtcaggaaaacaatgagcgcgaagatcttaccttcatcgaaaaggcgcgcttcgcacatcgcctgaacaggcagttttctcgagagattgtcatcgccgcgatgtcgatcgacaagagcaatttgtccaagatgcttctgctcgttgacgccctcccctctgaactgaccgatgctattggtgccgctcctggtgttggacggccgagttggcaacaacttgccgagctgattgagaaagtttcttcaccggccgacgtggctaaatatgctatgtcggaggaagttcaagcgctgccatcggcagaacgattcaaggcggtgatcgctagtctgaagcccagtcgggttgcgcgtggacttcccgaggtcatggccaccccagacggcaccagaattgcacaggtgacgcagagcaaggccaaactggaaatcacgattgacaggaaggcgacgcccgattttgcgaccttcgtgctcgatcatgtgccagcgctgtatcaagcgtaccacgctgagaaccaacggaaacggggagagtaaaccgcaaaagaaaagagccccctcaacgtcgccgtcgcggaagcccttctgtctctctagcgcgaacagaatcgcatttcctcgaatcctcgtcaagagtttttagcgccgttttggtgagctgatttcctttgcctgctgaaaggtgaaagatgatgcagacaggaagtgtaacgacgccattcgggcggcggccaatgacgcttgcgcttgtgcggcgccagacggcgctggccgatatcaaacaaggcaagacagcggacaagtggaaggtctttagagacgcgtccgcggctatggaactacttggaatccagtccaacagtcttgccgtccttgatgcgctattgagctttcacccggaaacggagttgcgtcaggaggcacagctgatcgtcttcccgtcgaatgctcagcttgcccttcgggcgcatgggatggctggcgcgactttgcgtaggcacatcgccatgctcgtggagtcaggcttgatcgtccggaaggatagcgccaacggaaagcgttacgctcgtaaggatggcgctggtcagatcgagcgcgcgtttggcttcgatttgtctccgcttctcgcgcggtccgaagagctagcgatgatggcacagcaggtgatggccgatcgagcagcattcaggatggccaaagaaagtctgacgatttgccgacgggacgttcggaagctaattacggcagctatggaagagggagcggagggcgactggcaagctgtcgaggaagtctatgtggaacttgtgggtagaattccacgcgccccgacgcttgctgatgtagagtcaattctcgaagagatgtggatgctccaggaagagataatcaaccggttggaaattagagacaattcagaaaataatagcaccaatgctgcccagagcgagcagcacatacagaattcaaaacccgaatccgttaatgaacttgaacctcgctctgaaaaggagcagggcgctaagccgagtgaaatagaccgggcaaggagcgagccgataaaagcgttccccctcgggatgatcctgaaagcatgcccgaccattggcaattatgggccgagcggtgcggttgctagctggcgtgacctcatgtcggctgcggtggtggttcggtctatgctgggggtcagcccgtcggcttaccaagacgcgtgtgaggcaatgggaccggagaatgcggcagcagcgatggcgtgcattttggagcgagcgaacttcatcaattcgcccgggggctatctccgagatctgacacggcggagcgagcttgggaagttttcacttggcccgatgataatggcgctcttgaaggctagcgggcaggggacgttgcggtttggctagaattagcgagtatggagcaggatggtctgtggtcagctgaccacagacctaataggttgaaaacatgagcgttttttggatgatcgacagaccatccgattcccggagtaccaagcgtgctctgatgggagcgataacattactcaacaagcacgaaggccccatgccgatcgttgatcgtgaaggagagcctgctctacatgcggcggtattttgccggccgaggcatgtagtcgcggagcactgcctatttactgccctaggcacaaacgttgactcttggatcgagctggcagacaaagcaataacccacacagaggacgattaatggctgacgaagagatccagaatccgccggacggtactgctgctgccgaagttgagccggctgctcctagaggtagaagagcaaagaaagcaccagccgaaacagcccgcacgggatcgttcaaatccgtgaagccgaaaacccgcggcctcagcaaccgagaaaaactggagaagatcggtcaaatcgaagctcaggtcgctggcggcgcaaccttgaaggacgccgttaagatcgtgggtatttccgttcagacctattatcaatggaagagagctgcggttcaacctgtctcacagaatccggccgtgtctgtttcagttgacgatgaactcggcgagttcatccaactcgaggaggaaaatatgcatggcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactatacgtaaccaactagtgcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagcgtaccgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttag

SEQ ID NO: 4 - DNA, 814 bp, Zea mays
  source 1..814: mol_type = genomic DNA
gtgagcgcttacacctctccgttcctccccgctgccttgcgcgcgcacgctttcgtgtagatctgggtcgccgcgtctccgtctttgtttagccggccgtagagcctccgctctaggtgcctcaagctcctcttcagttcttctagctccggtagggtttcccttttgcgcatagcgggggcggctgatgcacggttggctacctcagctattcgtcaggctactcgtcagtattttcggcgcactttgctagcttagatcatcgccgtttgttttgcgcttcgtccgtcgccgactcgacgaaccggccaatcaccaccctcgccgatccattactcttaaatccggaccacgccatatcggatcgaaccaaacgttcacgcgttgtcgactaaagcattgtgtggttgatttattaatcggctgatgcagtagaaatcctatagagtgtacatcagtttctagtttcaagcatgtaattgagcttgtgaggtgcagtattttgttgatgtacaaggcttagggcattgttttgctgaagggcctgtgatcatgtgtaatctaatgtggagtattaaacgtgtgtgtagggttgatgaaattatgctagtaatggctttcaagtaacacgtttattgtgatctgtgacatctttaaacaatatgcagtttgttttccttaaattcgttgctgaaattgtgacattggtcatgcttgagaaggaatcattccgtgaaatcaggttgcgatgcctatgataacacttgctggaatctgttgtgcttttctgatcactgttttttttgcaatccag

SEQ ID NO: 5 - DNA, 727 bp, Zea mays
  source 1..727: mol_type = genomic DNA
gtagtccttgacgcgttcgagcttgagtaggcgaaggcggcactttgagagcggcgccacggcgaggagccacgccgccgcctcggagcccttctgtcgcttctgcttccaaccgacacgggaaggggcggcgggtggttcacatttcttctctttcttctcgtctccctatttgcggtcgcccgggagaccgggctgcttgcccatcccgccaggagaaccatgtcccatctccggcggccgtgaattagggtttggctttgtgatctgtgtagggatctagctatatgtgatggtgcttgtctagatctatgtttgttaagcctggttcgttgatctggtgagatggtgcatgatcgaggtttgattgttttaatctagaatgttggatagagaattagaacagggattagccgaaattacgcattattttccatgatccaatgaagttagtagttatgtagcataattgttgttgatttatgccaatatgtgtgttagatctgagtgccctgaaatcgtttaaatgcactggcattcacgatctgtaaattttcataggccaaattcgtgttgcctgtttatgcccttgaagtatggatatagagttagtatgtttaatgtaaaacgatgtatagtacatctattcgtactattattagatattcaaaaaaatgaatacatattagcattagaccctttgaaccaattttattttgctatttag

SEQ ID NO: 6 - DNA, 834 bp, Zea mays
  source 1..834: mol_type = genomic DNA
gtacgcccgctctctcgctccctctctacctcctcgactccatctccttgaatttcactcgttactacaccgaccgctaccacccgcgctgcgccctgtgataatcggtgcaccgtgcgccggcggatgattgtattcttgaatctgccttgttgatagctcttggcgcccaccgagtgcatcaacaagacaacaagaaaccggtccctatacgaatggtattggctaccgtgttcagttcgtccgcgcggttgttgggaaaaaaaaatggacggtttagttaggctggtgcgcagccggacaggacaaatacacccgtgcggtggttaggcagcggagattgacgcatcattgcgacctcgcaaatggtccttgtctccctggtggttgtatgctctgttccaacatccgttgaggaactgcgcttggtgtgtgtggtccagttcctgcaattagcgtggttgttgttcgtcgcggcatcttcgtgggatttggtagatctgcaagaaagtgagtgctgcttttcaatggagttctttagcaatcagtgagatttggtttgccataggccgccttcttttcctgggtctactgattgagccctcccagtcccatgtgcttgccattaagcaacttgacaatcaatgaaaaggtgagggggtccgaaggagtaattacgaagcaaataacttgactgtgcagatttattgcactgaataacagaaatgacacacaaagtgtttttttttaaaaaaactttagacagaaaatcaaaaggatatacctctcctcttcgtaacatagaattcttatctcataaattcgttcgtgcag

SEQ ID NO: 7 - DNA, 982 bp, Zea mays
  source 1..982: mol_type = genomic DNA
gtaaactcctacgcctcccttcgatgcctccagcgtctccttcccgcgttccttagctgtagttattactgccttgatctgtactccctccgttttagttgtcgctggatagtgcaaaattgaactatccagcaacaactaaaaagaaacggagggagtatctatcttgttgtaggctctctcacttgtagctgcccgaataccttgactgttcttcacctcacgttagaaacacagggattccgcatgcctgttcgtggtcgctgtgcgcgtgagcctaaatttaggccgcatagagcatgcggcagctagattgtaccgagcggtgaaactttgccagtgcaaccaagagagggcatgaggcggcggcacgtccccgctgcacggctgctcgtggttggggcgagatctcgcgtgattgagtctgcgaaaaagcgcagctgcgatctagatcgacgtggttgctcgtgatctcgtctcccatccagatgtggtgaggcagttaggctgagatccggccccttgtgccatgtccgtgtggaccgagtcaggctggtgtcgttttatttgtactcgcaaagtgctgatgctacgatttttatttatgtttatgtatagagtgtaaaatacgatttttatttatatttatgtatagaatgtaaaacacaaatacatacgtgttgtgttggggtggatgcgaaattgagtttatagccaaaaattgggtttttatttgtacataattttaaattaaatgtgaatgagtatgatgatggtggaaatgaaaactagtgcgttttatatagtagagaaaagatagggagtaggtgggcttcatccagatctttcggttatgtcggggtttgtttggtcatggcaggcagggccacatgagatttctgtacggggtttgaattgacactgcatcgtcttcgactgacattatgtgctattgtatctatgaactggaaaacttttacag

SEQ ID NO: 8 - DNA, 856 bp, Zea mays
  source 1..856: mol_type = genomic DNA
gtaaggttcccttccctcctcccctcacacccctgttcgtgttccttcggatcggatctcagtggtgatgttagacgtccgcggctgcctacgtagtggcattgccgcccgaaaggtttgtttaggtggggtagatccgaaacaggccggatctggaccatgtccgcggcggggcggcgggacttgatcgcgtagctgtcgtgtgcatttctccctaccagtggcggaatcggcgatgtggacctaagggctaaggcttatctgctgccttgaccatttcgtcgctgacaaaaacaaagtgacaatcatgccgttctctgtttgtttatctggatcgttattacgctgtgaatcctgcgatatgtggctaagtgatttttcttctttttctgggggcagtttagcctttgacccagtcctaggtgtggtcactaggactgtgtagcatgatgagtgaggttgcagcaggctgattgctagtggacgtttttttccccaatttgttaggttttcacgctccaggttgtgcaagtaattttgctagtgattgtgtgatccatcttcaacgttgaaccttgtttttccccctaaaacccccaacaggaaatcttgccccgacttctattgcaaaaattgtaacgcttagcaccctgattgactcaattcctgtcactaggcatgctcggtcaaaagcagatgatttaccacttagaaactgccctgcccctgctttccacatagcatttcgaactttttgactactattgacacccccctaacttgccgaactatttctctcttcagctactatttacctagttataattacataaatgtttgtgtgtatcttgtgcag

SEQ ID NO: 9 - DNA, 1020 bp, Zea mays
  source 1..1020: mol_type = genomic DNA
gtaaatctcaaatttatcatgtattttcaaatgatataaaattcggatacgaataattttttgacattttttctctagagagcaaataatacataaaacaaattatacaaaattttattcttatttataataatatgttcaataacataagaaaaaatcagcatcaaatttcatatatatctatattaaaatattaaatttatgctaataatttggatacccattttatcatcatcttattccgatatggtatttcctgcaatcaaacacatgcccagggaacgctcgcacccgaagattggcgtaaccgaaccgatgcgacctttggcccgtttcaaaatgaaatgggccggtcaatgcgcggcccatccggccgatgatatcacgacccagcgtgcatcctatgaaacctctgccccggggctagggtttcctcccagccgtcatcgcttcacgtccgctacagaccggcgaggagacgaaaggtaagccacccactgccgccgctctcgtttcaccccatttcgtccggccgtctaattcgttggccggcccgttctgttcgtggaagcgctcggatctgctctgtatatgcttgttctcgttcggttttgccggtcggagttgtctcgcgaggcgtagcttgtgcgtcaggctctcttttgccttgtagatttcctgcatctgctcttactgatagcttttgctgttaatcttgagagagttatgccacgagtctctttgatattctattggggtaatgatatgtggggatactgtaatgtttctgatattcttgctatggttggtttgggcatgaagtagaatttgtaatggtgcttttaggatcctgtttttggtgctaactctgtcaacactagactgagaattgttgttaacaagtagctttcatgttgcacgacaagatccttctgtgtttcattccagttctcatgattttatttcttttttgcatcttcatccgcctgacataatggtctttatctacttgcaattcag

SEQ ID NO: 10 - DNA, 841 bp, Zea mays
  source 1..841: mol_type = genomic DNA
gtatacaaccgacctcgtctcccccgatttctcaactacgtagtgtatgtacgcatcggtaggtagatgggaatttcgggccactggtttggggggtttaatttgcgctatcgtttcggtttgcctgtggtttcggaggtagatttgggtcgcaggtaggttgtcgcttggatctgggagaggcgaggagctaaattcgcatagcttgtaataactcaccccggttgctatgaaaagccgtaggccgtagctgctgctgctgctgtaatttactacttattttcttctaatataggggattcccttcctgcaacttttttttaaatacggttcttggttactggctggcttagtgcagtgggaccttgttgccatgaatgattgttgcgcaatttagtagatcattagattagcacgacgtacctaatcatgggtcccgtgaattttagcctagtccccattatttgccccttagtcacgcatgtgtttggtgtacttcaaggaatctgtccatatgcatcggatctatgggttcggccatgatgttgacattgaactgtggccgttcatgttcgactttaccttgcgcccgagcaaaaagaaggataaatcgtgtgtaccaatctggctatacggcagctcgatatgtctgaatgaagattgggagtattcttctgtttatttatttgtcaatttttattctgaatattcatttgttctccagtttagtagtgcatcattaacacttcaattctaggtctattgctatggtatagtagcactctttcaatctttcatgtgtacagctgatgccttatgttgatccccttccttgctttag

SEQ ID NO: 11 - DNA, 1044 bp, Zea mays
  source 1..1044: mol_type = genomic DNA
gtccgtgccgggcggcccggatggacatctatagcccgcaggtcatggatagatagataatttcgcgtccgtgataaagccggactgctacgtttccgacgggagtaattatttacacactcttcagcaccaacggcatctcgtggacgctggatcccaataccaaattgctacaagctcaatgatgtctgggtgggcttctggcccatttagaatatcatctctatacgtctttatacagaacgaaaaaaaaacgagcggccaagatggtgttggagtatagatcggacggcaaagccgtgctctccgccttttaaaaggctgtccgactcgacccttctcctctccgtcgcatttcccgtccggtctttccgttacccggcggctttaaaccctagttcccattccatcttcgtttccgctccgccgccgtcgatggagttctggggtgacctcccttactttctctctcgcaaactcaattctccgtgatacctgcgctttcttcgttttgttgcggttcctctgtttgatttttgggcgttttagccttttatttaaggccctgggaatcatgggcatggtttgattggctactgctagcacttgtttgcgaatgttttagggccctgtgaatcgtggctattgtttggttcgctactacttactactagggcatcgttgttctacaatcagcagttatgtggcattgcctatttgcacagtatggaaaaaagtagattaaaatggttgaaatcatacgtggccgtgtcctgtttcaatagggtcttattataggacgtgacggtgatgccactgaattaggatgtttgttcttttgtaccattgttaaacagcaccagatcattcttaatgtaacctacatcagattgtttgttttgttctactacaagtatgttcagcatggcaggaggaagtgtgctcctaaatctgttgtttaggcgcattgcgaaatgtttgttctaacagcatcaggtttgcattcttaatttaacctatcttgtattgctctgtcttttgcag

SEQ ID NO: 12 - DNA, 648 bp, Zea mays
  source 1..648: mol_type = genomic DNA
gtaagcgacgacaacgagcagtgggcggcaggattgagcccccccataccttctctctctgggtttcgctctctctcgcgtgctcagaaagtttcggaggcggcggttttcgctctctctcgagcgctccgtccaagcgtttttctttcagaaccagtcctttttgattccagactaaaatttattgcttttattattagcaataaatttcatggttcaaaaatgcgacagtttttttacttgaatagagacatttattttaaccaaagaaaaatcatatatatataataaaaacaaattagtggcatccatacatatggatgcccaaacaattatcggttgaggaagtcaacaggaaagtatcttaaaggttggaaacgtggcaaatcgaggagcatgagctggtagggcactcgcagtgggtggagccggtttgctgacgcggaaggaaccgaccattcatgactcgcttggcacgcggtgaggtgagtgtgagtgtgagtcagcctctcgatctgggttgggttttggtctataaaataccccggcctccatctcttctctgggttgtggttgtggcctcctatccttgcacgcacacgcaaacgcatcccatcctgtcctcgcgttactactagttagttagag

SEQ ID NO: 13 - DNA, 632 bp, Zea mays
  source 1..632: mol_type = genomic DNA
gtgaggagccttctctctctctctctctctttctctgtctctctctcttcctaacctttcttcccttcgtcctcgtcccccacgctctgctcttgtggaattttctattagcgtctgccgccatttgtttcggctactttgtgcgcgcgcaagcgcaaaccacgggggtctctctcgtgttcgccattctgccgaatcgccactgcaagctcttctaccgctttctgtgtgctctgacatctggactccggagtccggacgtccgcggctctgtttgctgcgcttgttttcttttttccagctatgcttcgtttcttctcgaattccatttttttatctctcttttttccctcgtggacgaagcaaagcaagcaagacgaccttgcatctgagactctgagactgtactgtttcttttgccattgggtttttccctaagattcctttttggctgccaatgttcagtccgacagcagcacccgctgcaaccatttcagcacttcttccgcctctgtttccataatatttcttcttttttttttccatctctttttttttgtgtgtgctatagcttttgcttgactgaaacgcagcacacaccttacacaaccaaacatttttttttggcgcag

SEQ ID NO: 14 - DNA, 1405 bp, Zea mays
  source 1..1405: mol_type = genomic DNA
gtatatactctctctctctcatccgcctcttgattcatgcggcccttgtgatgtgtgtgatcttgttacttggccggctgtggttctttttgtgtctcttgattcatgcgggtaggattaatctttgtttaatccgcaaaaaaggccttgattattcgatactatctatctgtaatccgcaaaaaggcgatgaactttccatgttctcggtagtgactgttctgtatcgattctgctgtctcatcaactagtcgtcggtcgcaaggactcgatcgcacgtccatttcgacggtagctacaacaagcatttcctgcagaataatatctcggttttatttctagaaggacggtcgttgtagtttatttaaagggaaaaaaagatcagttgtagtagatatgcgctctagagtccagaatggcttaaaggacgatggttacggatggaaaccttttcctctcatcgcgtgccgatagcgaaacggaagcttttgtcagtccagggaggggaaaaaatttcaaggtgttgagcagccccttcgttggatatagcagagcaggcgacgtgcaagacaattaaagcaatgcctacgagagcggaccctgatatttttgtatgtgtgattgtgatacgtgtctggctttaggctttggaccgagaaatagctcgcttttccacaggggggaaataaggtgccccttggaaatgacagtcggtattgggttcagaaccaggctttgagattacaatagagaaactggcagcttgagctgacgtagcgttttatcagaagtcgtcaatatcgcctgctacgtatatcttatagtttaattgttacttgggacatcggtctctagcttgtctggacaactggttctcctcctcctgatcggatttgattttttttacgagccgtcaaatgcatggtttgctataggatattcatatttcagagacgtctatcgcttcttagcctaccaccgcaactctgctgtggaccaccattattattggttctcctcatcgggcttcggggttcacaagtctgcatattcttctctagttttttctttgctagctttcaggctgttttcaatgctacttcagacagagacgtcaatttttgtctgacgcctattgaggaccgcgggtatagtagggttttcagatccagagcactaaaccaactactacccggccgccgccgccgccggtacatgtaaaaaaaggcttcgacctgcgagatgaacgcaacggagagcattcaacaaaccaaaaccgcataaccaaaatgttcagactagcgcgtggtagtagtagtacttgcagtcatgtcatgcagtttgccacgccgccgcgtgcacgcagtttgccacgctagctaatctgttctcattccgctcgcag

SEQ ID NO: 15 - DNA, 1361 bp, Zea mays
  source 1..1361: mol_type = genomic DNA
gtaagcaggagagagagctctttgagtttgatcgatccttaagacaatactgtagacttggcacaaccatgcccccgtccatatcccgaccgtccgattgctgaatccacccccgcatacctcatctgccatccattctcgctcccgccctcgaccccgccgccatgcgcgcagccgcctccgcccaacaaccatgcgggatcctcgcccaacagccatgcgcgccgactacgcctccaccagatccagccttcctatagtcatcctgtcatccaacagggatcctggccacatagagtttcgcccgaatcgatcgccgattgactccgctagggttcggcccgatcgtcgcttcgtcctctcggctcccgcggggaccccgccgagatgtctgaccggagctcgccggctgcgccgacgttcttccttttggcccgcaggccgaggcatgggacgtcaccttcaaggtgaggcatccgatcgatttttctttctttctttactacactcctttgcgatatggggacgacactcggtagtggcgtgaggtgaggtaaatcgcgttagtttagttgtagggtttgatcgcttcaggggagaccaggggttgggctttccgtgttgaaccgtcaatcggacgtagtagtagtgcggattcggggtttgatcgatggaaagaggggttgtccgcactcttggtgtggttatagggttttgcgatttgtttgtctgtgtaggcctgtttcgtctcgaggagtagattttcattgctactaacaatccctatgtggtttggtgaacacgtattttggtctgtatatggtttaaacgtgaagactatggtagtgtgagaccatgatttggatccttttctgtggcattatagttaaaatcgtgaggatgcacctatatctatcttttagcgcttagggtattgttatagacgagatcccctctttgggctctaaaaatagcaagaaaaagacatcttttgggcaagttaacgtcctgtattattctgaacgagatatgtttactttcttataagtttgatgttttggtctggaatatggttgcgttcatcttccaattagtgtgtttgcagtatgtgttggtgtagtttctctgtgggcattttgtggccacagaaatgatagattttaagaaaggtttaggtagaagggtaccttaagtgttgtccagtacaaagtaacaatttgtagcacttgtttcttttcttttgtttgactatatgaaatttcggccatgtaattgtttcaaaataataagatcgaatagtgttgcacactacttcccagtcctatgtatacttataagatttttcctctttgatatttcag

SEQ ID NO: 16 - DNA, 703 bp, Zea mays
  source 1..703: mol_type = genomic DNA
gtgagatttcgagagattcgtccgtaccagggcttggatctacgctccggccaccttcaggcttcggttcgccgcccaccaaccgagcctaggagtccgccattcattcaaacgccctcgacggcgactgcgcatcggttcagatccaggcgtcctttcaccggcagctccgcctcccatgcaatcttgctcgttgttgttgtcccctccctggactcgggtgtactggaagtccatgcggataaaagaaactcttttgttggtatggacgccataatgcgttttgttgcggaatttttttgcggcttggcgtgctgtcaataccggtttagttttccaatttttttgcagggttcaaccaaacctcctgctgacagacccttctctgttcagtttgcttacccaactgacttttttttcttgttcatattctagttggatgctgagtggcatgccggtcgatatttgggaaagcagatttttatgttggcaagtgtgagtgcgagttctttgctgaaactttaagcttcacctgagatctgatattgtcggtgccaaattgctgtacatttgactatttgaggacacgttcttaggtaaatcattggaagacatatttcacttcgcgtaggacacgtacttctccaagatgatgccttcacctgtctcaaacctttgtgatttttatatactcgcttggcag

SEQ ID NO: 17 - DNA, 1341 bp, Zea mays
  source 1..1341: mol_type = genomic DNA
gtgaggccgcgaccaagaaggcgtaggcggcggcgctggccgtggcggggcggtggcacgatacgctggccggctgggcgacgccgtagcctgtaggtctctctcgcaggaaaaagtttctgtgttccaatggaagtaagatacaaccgttggattctaatggaagtaagatcgaacggttgtgatagatacaaacgaaactgaagatcttttatagtatacatagatagatcgacttaagcaaaaaaatcttaggccccgtttgtttcgttggattgaattccattttgataattataatttagtcaaaactaattaagtttatatatttatatatacgatatatttgtatattatcctaaatcatacgagagagatagttatatactatatttatgttatagcgaaacaaatagatgagtgtgctataagttgtacatcggaaaaatagcatgtaaatctatagaatcaatttccatctctcaccccattaatttgagataggcttatatgataactttggaaagttgtggaatgtcacattcttttaaaaaaatagactattttattagtaagattcaaatttctcgaaataaaagaaaacaaacgagaccttaaagataatgttcctataacaatctaataacaactcaaagagtaagaaataaaaaaagtaacggcgtgtttggtttgcaggttggactgcttctggagtcatccggacctatgtccgagcctacattatcatttggtttgaatcgcggaacgatgtcgtccgtcactgcgttgttctaataatatactaacacatggaattagctcacttcgcaagaaagtgcaagaccgcttcgtccggagccaggccacgatggatgagtcaaaccatcaaaccaaacacgctgtaataattccgaaaccgcccgcggagcatcgcagctactgacaagtgggttcggaaggggatcccgtgtcgtgggtccacacgtcaccgtgtgcggcgtgctctaactgcccgggcccggccagtggcgggtagggggggagagggactgagcctgcataaatcgtcagcgaataggccgcccgcacgacttctcttcccaattcccatagatcgatcgccgacccctcgagcaacgcgatcgcccgccgacccgacggcggcatggacaccgagtacgtcgaccaccccgcccgtcgccgccgcgatcagacggcgcatctctttttcgcacgcgggggcctttttcctttctctatcccccatctttgtcgatttcttttatttttcttccccccttgaggatgatgatgatcgcctcgggccgtcggctcctgcag

SEQ ID NO: 18 - DNA, 1125 bp, Zea mays
  source 1..1125: mol_type = genomic DNA
gtactaataaactgatgattcattcattcatggcacggcctaccaatgcaaataaatctatctcacgctatgagaagaaaaccgagagagagagagagagagagagagagggcttgccttcccggccggccgttagtgctcaattgggcacgcgattaccgaggcaagcagaggcctcgggtcgggtggggcttgtttgacggggacggcagatccacgtctctgtcacgtgactccaggcggtggtcgcttgctccatgtgccgcgtcgcatcccgatctctggctgtggttgcctggctggcaagggccaaccgcccgtcgtcagcgatgaggatgcttgtagtgtcgtgtcgacttttgcaaaaacaacgtgcccagccctgggtttgtgcgccgccgcaccagccaaaacaaagacgaaaccgaaagactcgtcaaaaggcaaaaccaagtgaggaaagacaactgaccatagcaaaaaacacaactttgctagttggtttccacgtatctttgccgcatgaactggtcccggccgtcacgtttgcttatagttccaccacaataatagtcgacccgtggtcccgttggttttgattgagagtagagagcatccaccggacagttaaaagtgtgtttcattgttttttcccctaataatctagtacttattctctacgtccacataatttagtcgttttgggtttattctaaattaaactattttaattttaatcaacaatatatataattaaattattttaaacttaaaagaattatatattatgatagtttaattcatgataaatttagtaaaattacttttgtattgtaaaaccttataaaaggttcgatatatatataactggtcaaaattgatagggaacgacttagaacaaacttaaaataactaaattaaactaaattatatggacgaaggggtaggtacctactccacagtacttaatttcctctagtagttaaccgaaacacggtagatactgaatgaatgtgttgcaagaaacatactgatctgtctacttttgcttctcccctcttcgcctcttcaataattcggtgtgcaaagatgttgagaagagaaccgtgaataccgattttgcag

SEQ ID NO: 19 - DNA, 894 bp, Zea mays
  source 1..894: mol_type = genomic DNA
gtgaggcatccgatcgatttttctttctttctttactacactcctttgcgatatggggacgacactcggtagtggcgtgaggtgaggtaaatcgcgttagtttagttgtagggtttgatcgcttcaggggggaccaggggttgggctttccgtgttgaaccgtcaatcggacgtagtagtagtgcggattcggggtttgatcgatggaaagaggggttgtccgcactcttggtgtggttatagggttttgcgatttgtttgtctgtgtaggcctgtttcgtctcgaggagtagattttcattgctactaacaatccctatgtggtttggtgaacacgtattttggtctgtatatggtttaaacgtgaagactatggtagtgtgagaccatgatttggatccttttctgtggcattatagttaaaatcgtgaggatgcacctatatctatcttttagcgcttagggtattgttatagacgagatcccctctttgggctctaaaaatagcaagaaaaagacatcttttgggcaagttaatgtcctgtattattctgaacgagatatgtttactttcttataagtttgatgttttggtctggaatatggttgcgttcatcttccaattagtgtgtttgcagtatgtgttggtgtagtttctctgtgggcattttgtggccacagaaatgatagattttaagaaaggtttaggcagaagggtaccttaagtgttgtccagtacaaagtaacaatttgtagcacttgtttcttttcttttgtttgattatatgaaatttcggccatgtaattgtttcaaaataataagatcgaatagtgttgcacactacttcccagtcctatgtatacttataagatttttcctctttgatatttcag

SEQ ID NO: 20 - DNA, 33 bp, synthetic construct
  misc_feature 1..33: note = intron_TS1 fwd primer
  source 1..33: mol_type = other DNA
caagcgatcgcaggtgagcgcttacacctctcc

SEQ ID NO: 21 - DNA, 35 bp, synthetic construct
  misc_feature 1..35: note = Intron_TS1 rev primer
  source 1..35: mol_type = other DNA
tcgggtacctggattgcaaaaaaaacagtgatcag

SEQ ID NO: 22 - DNA, 33 bp, synthetic construct
  misc_feature 1..33: note = Intron TS4_forward primer
  source 1..33: mol_type = other DNA
caagcgatcgcaggtagtccttgacgcgttcga

SEQ ID NO: 23 - DNA, 32 bp, synthetic construct
  misc_feature 1..32: note = Intron TS4_reverse primer
  source 1..32: mol_type = other DNA
tcgggtacctaaatagcaaaataaaattggtt

SEQ ID NO: 24 - DNA, 33 bp, synthetic construct
  misc_feature 1..33: note = Intron_TS5 forward primer
  source 1..33: mol_type = other DNA
caagcgatcgcaggtacgcccgctctctcgctc

SEQ ID NO: 25 - DNA, 29 bp, synthetic construct
  misc_feature 1..29: note = Intron_TS5 reverse primer
  source 1..29: mol_type = other DNA
tcgggtacctgcacgaacgaatttatgag

SEQ ID NO: 26 - DNA, 33 bp, synthetic construct
  misc_feature 1..33: note = Intron_TS6 forward primer
  source 1..33: mol_type = other DNA
caagcgatcgcaggtaaactcctacgcctccct

SEQ ID NO: 27 - DNA, 29 bp, synthetic construct
  misc_feature 1..29: note = Intron_TS6 reverse primer
  source 1..29: mol_type = other DNA
tcgggtacctgtaaaagttttccagttca

SEQ ID NO: 28 - DNA, 34 bp, synthetic construct
  misc_feature 1..34: note = Intron_TS7 forward primer
  source 1..34: mol_type = other DNA
caagcgatcgcaggtaaggttcccttccctcctc

SEQ ID NO: 29 - DNA, 30 bp, synthetic construct
  misc_feature 1..30: note = Intron_TS7 reverse primer
  source 1..30: mol_type = other DNA
tcgggtacctgcacaagatacacacaaaca

SEQ ID NO: 30 - DNA, 35 bp, synthetic construct
  misc_feature 1..35: note = Intron_TS8 forward primer
  source 1..35: mol_type = other DNA
caagcgatcgcaggtaaatctcaaatttatcatgt

SEQ ID NO: 31 - DNA, 33 bp, synthetic construct
  misc_feature 1..33: note = Intron_TS8 reverse primer
  source 1..33: mol_type = other DNA
tcgggtacctgaattgcaagtagataaagacca

SEQ ID NO: 32 - DNA, 33 bp, synthetic construct
  misc_feature 1..33: note = Intron_TS10 forward primer
  source 1..33: mol_type = other DNA
caagcgatcgcaggtatacaaccgacctcgtct

SEQ ID NO: 33 - DNA, 29 bp, synthetic construct
  misc_feature 1..29: note = Intron_TS10 reverse primer
  source 1..29: mol_type = other DNA
tcgggtacctaaagcaaggaaggggatca

SEQ ID NO: 34 - DNA, 35 bp, synthetic construct
  misc_feature 1..35: note = Intron_TS11 forward primer
  source 1..35: mol_type = other DNA
caagcgatcgcaggtccgtgccgggcggcccggat

SEQ ID NO: 35 - DNA, 32 bp, synthetic construct
  misc_feature 1..32: note = Intron_TS11 reverse primer
  source 1..32: mol_type = other DNA
tcgggtacctgcaaaagacagagcaatacaag

SEQ ID NO: 36 - DNA, 33 bp, synthetic construct
  misc_feature 1..33: note = Intron_TS12 primer
  source 1..33: mol_type = other DNA
caagcgatcgcaggtaagcgacgacaacgagca

SEQ ID NO: 37 - DNA, 28 bp, synthetic construct
  misc_feature 1..28: note = Intron_TS12 reverse primer
  source 1..28: mol_type = other DNA
tcgggtacctctaactaactagtagtaa

SEQ ID NO: 38 - DNA, 33 bp, synthetic construct
  misc_feature 1..33: note = Intron_TS13 forward primer
  source 1..33: mol_type = other DNA
caagcgatcgcaggtgaggagccttctctctct

SEQ ID NO: 39 - DNA, 35 bp, synthetic construct
  misc_feature 1..35: note = Intron_TS13 reverse primer
  source 1..35: mol_type = other DNA
tcgggtacctgcgccaaaaaaaaatgtttggttgt

SEQ ID NO: 40 - DNA, 34 bp, synthetic construct
  misc_feature 1..34: note = Intron_TS14 forwrad primer
  source 1..34: mol_type = other DNA
caagcgatcgcaggtatatactctctctctctca

SEQ ID NO: 41 - DNA, 36 bp, synthetic construct
  misc_feature 1..36: note = Intron_TS14 reverse primer
  source 1..36: mol_type = other DNA
tcgggtacctgcgagcggaatgagaacagattagct

SEQ ID NO: 42 - DNA, 43 bp, synthetic construct
  misc_feature 1..43: note = Intron_TS15 forward primer
  source 1..43: mol_type = other DNA
cctccgcttcaagcgatcgcaggtaagcaggagagagagctct

SEQ ID NO: 43 - DNA, 41 bp, synthetic construct
  misc_feature 1..41: note = Intron_TS15 reverse primer
  source 1..41: mol_type = other DNA
aggctaagttaaagtcgggtacctgaaatatcaaagaggaa

SEQ ID NO: 44 - DNA, 34 bp, synthetic construct
  misc_feature 1..34: note = Intron_TS16 forward primer
  source 1..34: mol_type = other DNA
caagcgatcgcaggtgagatttcgagagattcgt

SEQ ID NO: 45 - DNA, 34 bp, synthetic construct
  misc_feature 1..34: note = Intron_TS16 reverse primer
  source 1..34: mol_type = other DNA
tcgggtacctgccaagcgagtatataaaaatcac

SEQ ID NO: 46 - DNA, 42 bp, synthetic construct
  misc_feature 1..42: note = Intron_TS17 forward primer
  source 1..42: mol_type = other DNA
cctccgcttcaagcgatcgcaggtgaggccgcgaccaagaag

SEQ ID NO: 47 - DNA, 43 bp, synthetic construct
  misc_feature 1..43: note = Intron_TS17 reverse primer
  source 1..43: mol_type = other DNA
aggctaagttaaagtcgggtacctgcaggagccgacggcccga

SEQ ID NO: 48 - DNA, 43 bp, synthetic construct
  misc_feature 1..43: note = Intron_TS24 forward primer
  source 1..43: mol_type = other DNA
cctccgcttcaagcgatcgcaggtactaataaactgatgattc

SEQ ID NO: 49 - DNA, 44 bp, synthetic construct
  misc_feature 1..44: note = Intron_TS24 reverse primer
  source 1..44: mol_type = other DNA
aggctaagttaaagtcgggtacctgcaaaatcggtattcacggt

SEQ ID NO: 50 - DNA, 46 bp, synthetic construct
  misc_feature 1..46: note = Intron_TS27 forward primer
  source 1..46: mol_type = other DNA
cctccgcttcaagcgatcgcaggtgaggcatccgatcgatttttct

SEQ ID NO: 51 - DNA, 47 bp, synthetic construct
  misc_feature 1..47: note = Intron_TS27 reverse primer
  source 1..47: mol_type = other DNA
aggctaagttaaagtcgggtacctgaaatatcaaagaggaaaaatct

SEQ ID NO: 52 - DNA, 100 bp, Zea mays
  source 1..100: mol_type = genomic DNA
gtccgttcccgtcccagatccgtccatggcttcgtccagatctgacctgtcctgacacaccctcacccggatctgtccctccttcccctctcccctgcag

SEQ ID NO: 53 - DNA, 147 bp, Zea mays
  source 1..147: mol_type = genomic DNA
gtctgcttcgtcctcgctaggtttcatttcgcggtctgtttgtgccgttggggctagatccgggtcgtggttcaacagatctgcttcgttttggtacagatctgcgttcgctcgaatcgagcatgacgttttcatgtgattatgcag

SEQ ID NO: 54 - DNA, 82 bp, Zea mays
  source 1..82: mol_type = genomic DNA
gtgcgtgcatgcgcacgctctgcttctgcctccctttcccttttcctccgaaagaactgaaacggaacgcatcttcgctcag

SEQ ID NO: 55 - DNA, 85 bp, Zea mays
  source 1..85: mol_type = genomic DNA
gtgcgtcactgtccaggtgcttggcttggatcagaatattgttggcggtgacactgtcttctctcgatcgatcgatcgatgacag

SEQ ID NO: 56 - DNA, 107 bp, Zea mays
  source 1..107: mol_type = genomic DNA
gtcggtttccaatctgttgaccatggatccacagatcggagcagttctttcatagtactcagcgatctgtttgggtcctaaatttcctttccccggctgttgtttag

SEQ ID NO: 57 - DNA, 604 bp, Zea mays
  source 1..604: mol_type = genomic DNA
gtatgtttcgtttttatttccttcgtgtcatatttctgggtgcgagttttgtgcctagatgattcgtatgtgttcgagttggcggtgctctaatctttgttttaaggttgttatatggcatgttagtgtcatcaacgattcatgattaatagactcagtagctaccatttcatgatttatgcagcgtatcagaggcaagatataaatcttgggttcaaagttgcacttgattagctgatattatttttgtattggctagtccatgtttttggttggaatttagtcttgaatgatagtgttgcatccggtttgctctatgtttaagccgctacacacctgtgaaggcttgtgtgtagtttctagaatcagtattttgacaatattacagtcatattgcaatagttgcatgtgctagtgtaagaattgttctgttcattttttatacatgctttgttctcttttgttttttcatcaatgaaaaaaacataaaagatacagtttttttatttgtctaaatatgggtgggttaacctttcaccctgctggtcatggaatatgtgttttcaattacttatctgcaacttgtggatgcggactctttcag

SEQ ID NO: 58 - DNA, 659 bp, Zea mays
  source 1..659: mol_type = genomic DNA
gtaagtatcgattgctgattgctgcggatcgcgtaccgtggtcacgctgcttagttcagctctaacaactgatcgctcctcctccactgttaccgattaatggcttgatcggtgccgagtctgttttaggtcgtgcccggctctctgctcgggcggcacgtgtggtccggtgttgcagcggatgtagaattttgaccttgttctctagctgtgaatgacagtattataggcacagacttatagattgatgtgcgttttgcgttgaactgctcatcgaacagatgctcccaattcggtagtttatggcttgtttggatacatgcgtgaagttttcagctgataagattttaggaaatgttgtttttggcattaagtgtttttataatgtatgggtctaagggaggcccagagtgtctaccttccgttattttgatctctgaattgccgcctttcacacgaagggcggtcacgcgtgtcacgtgaagggtggtcacatgatctgcagcatagcactactagatgttggaccttagtgtatctatgcacaaattttctgattagatacttgctgaaagctatttcttcttgcgctatgatggaccatactagctattttgtgattctagcgcccttacaaaatatttatcactgctttgcag

SEQ ID NO: 59 - DNA, 9794 bp, synthetic construct
  misc_feature 1..9794: note = Vector Sequence
  source 1..9794: mol_type = other DNA
ttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttcatggagctccaaataatgattttattttgactgatagtgacctgttcgttgcaacaaattgataagcaatgcttttttataatgccaactttgtatagaaaagttgggccgaattcgagctcggtacggccagaatccggtaagtgactagggtcacgtgaccctagtcacttaaattcggccagaatggcccggaccgggttaccaaaaaagcttcggccggggccgcactgtcaagctattattagcttctttaataagtccaatgtgaacaaaccgtctagggttagatggattgctttcacagatttccttactggtctaggaatccctgtaaatatagagcacatagatggaaaaaataaccatctggctgatgctctgtccagattagtaactggttttgtttttgcagaaccacaatgtcaagacaagttccaggacgatttagggaaattggaagcagctcttcaggagaagaaagaggctccgcaagcaatgcacgtagaatatgtctccctgttgatcagatcagcggaccgcattacccgctcgctctgctttatgagggactcgtctcacagcagaatttactcatgcaggccaggcaaagaaccaatgaaggccttaatctgcgaacagaagtcatgccaatccaaaggcgacttagggaatacgaggactgtgcactccaagagtgcattcaatcagcaagacaactggtggccctccaccagcacaaactcgcttacatcagaagcaaagctacaagggacaacgcatatgccgataggctacccacatgcaatcgggaccacgagcaactgtgtgaagtggtcgagctattagaaggaatctcggaaagaatcagcgatacagctgtctaggacagctggcttcaattatggagcgtgatggacccccccgcaataatccaaagtttggtgtgcttttagtagtgcgtctttatggaccactactttattgtaataatcgatgctttttgtagtgcgctcttcgtgcgctctactttatgcttttgcttttgtaagtgcgctgtaagtgcgcctgtctttcttcagatgcttatcctttaagcatcttttgctttttgcgtggcatcctttagttcacaatttaaagaatgacgatggggcccaagatgtgcacccggttctctaaattgcctatataaggatatgccatagccttgtttttgcaagtcaggaatacctgagcataacttggctaagcaaaagtttgtaagtgttctaagctttcatttgtaaactttctgtttggttttaataaaatctctcgtcaatcgttgtgaacatatattgtttgtttgtattgttgtatcttatttgttgtggtgataaggatcttcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggcgccttgggatccatgggcaacagcgtgctcaacagcggacgcaccaccatctgcgacgcctacaacgtggccgcgcacgacccgttcagcttccagcacaagagcctcgacaccgtgcagcgcgagtggaccgagtggaagaagaacaaccacagcctctacctcgacccgatcgtgggcaccgtggccagcttcctcctcaagaaggtgggcagcctcgtgggcaagcgcatcctcagcgagctgcgcaacctcatcttcccgagcggcagcaccaacctcatgcaggacatcctccgcgagaccgagcagttcctcaaccagcgcctcgacaccgacaccctcgccagggtgaacgccgagctgaccggcctccaggccaacgtggaggagttcaaccgccaggtggacaacttcctcaacccgaaccgcaacgccgtgccgctcagcatcaccagcagcgtgaacaccatgcagcagctcttcctcaaccgcctcccgcagttccagatgcagggctaccagctcctgctcctgccgctcttcgcccaggccgccaacctccacctcagcttcatccgcgacgtgatcctcaacgccgacgagtggggcatcagcgccgccaccctccgcacctaccgcgactacctcaagaactacacccgcgactacagcaactactgcatcaacacctaccagagcgccttcaagggcctcaacacccgcctccacggcaccctcgagttccgcacctacatgttcctcaacgtcttcgagtacgtgagcatctggagcctcttcaagtaccagagcctcctcgtgagcagcggcgccaacctctacgccagcggcagcggcccgcagcagacccagagcttcaccagccaggactggccgttcctctacagcctcttccaggtgaacagcaactacgtgctcaacggcttcagcggcgccaggctcagcaacaccttcccgaacatcggcggcctcccgggcagcaccaccacccacgccctcctcgcggccagggtgaactacagcggcggcatcagcagcggcgacatcggcgccagcccgttcaaccagaacttcaactgcagcaccttcctcccgccgctcctcaccccgttcgtgcgcagctggctcgatagcggcagcgaccgcgagggcgtggccaccgtgaccaactggcagaccgagagcttcgagaccacactcgggctcaggagcggcgccttcaccgcccgcggcaacagcaactacttcccggactacttcatccggaacatctccggcgttccgttggtggtccgtaacgaggatctcaggaggccgctgcactacaacgagatccgcaacatcgcttcgcccagcgggaccccaggtggagcacgggcctacatggtgtccgtgcacaaccggaagaacaacatccacgcggtccatgagaacggcagcatgatccacctggctcctaacgactacacggggttcacaatctctccgatccatgctactcaagtcaacaaccagaccaggacgttcatctcggagaagttcggcaaccagggagactccttgaggttcgagcagaacaacacaactgcccgctacacccttcggggcaacgggaacagctacaacctctacctgcgcgtcagctccatcggcaactcgacgatcagggtcacgatcaacggaagggtctacactgcgaccaacgtgaacacgacaactaacaacgacggcgtcaacgacaacggcgctaggttctccgacatcaacatcgggaacgttgtggcaagctccaactcggatgtccctcttgacatcaacgtcaccttcaactctggaacgcagttcgatctgatgaacacaatgctggtgccaactaacatcagccctctgtactgatacgtagttcgcgcctaggtttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaaggtacatagtacccttgtacttttggtttcacctgcatagagaaaacccacaagaatatgacagtctgatttgtcagtctcattctcaagcaacatttctctatccgttactttcatggtgaataacacaatccatcatcaatactttgtgttactcagaaactgaaagttattccgagtcttgcatatctttggacctactcgtttttctaccattattgctgattgttaagctctcgctacttgaatcggcattgttggagtgggaaggttcaaaaaattggagttatgactagttgtctctttctatgtacgatggagaaaatgaataaacaactgagaaaatggctcttgtttagttgatgatgctcttaagctttccactggttgccatatatgatttgggcatttcactttgatcttaatgggccttgtaaagcccaagactcatgattatctttagttgatgctcttaattaggtgtgggcaaataattcaaactgtatgtacccgaccaaaaccaaagcaaaaataatcgaaccaaaccgaaaatttaaaaataaccgaatgaaaactaaatcctataactgaaagaactgaaaccgaatcaaaatatttaatgtaaccaaaaatatccgaaatataattatattgtcaaaaatattaataatttctagattaaataattaaaaatacttaaaaatttatataaaatagtaaaaatactcgaaaataaccacaaatattcaaaaacaaccgaaatatcccaaaatattcaaagcaaaataaccgaatggataccaaattttaaaaccgaaaaaactggaacaaaaccaaaatcgaaccaaaatttcaaaaatcgaataaatactaaactttagaacaaaaaaaaacgataaccgaatgtatacgaaccaaagccgaattagataaccgaacgtccaggactactcttaatctttccgccacttatgatttgggctattactttgtttataatgagccttttcaagctcaagttcatgattgtccgtgagatgagaaactgacttgttggattcgaaaccctagctagtattggttaatacttaatacataaatgacctgcattgacatcatcatccaagaaaataaaaattgtatgcttgagatatttagttttcctagctaggttttctttattttagtaccgaatctttaggtgtgccacgttaatttagacccattttttcatacttaccaactgagtctagtttaatcatgactataatcgtataaaatgattcagtcgacgtcattgcgaacgtatataaaatcatccaaattgacgtcattccaaagaggtaagcatgcttatctaagagtccgagcatactaaacaagacgacattttatttgcactccaaatcaaattttgtattgcctaaagaaaaacaatcaaactcaagtttcttaaaattaatttcattcaaactaatcactttcaatatctcacatattattcatgccatttctatttgtctaaacatgatttaaaaaaaaaagtaaaatacaaagattactatgcaaaaactctataaaaaaaaattcaaatttcttatttatttgtgacatcaaattttcaaaataatttttttaattatcggttgatccggtcagtcgataaaaacataaactttcagcgaccgttaaaactttcctactaccgatttagagaaaatcttagcttgaaacgtaattgtaacctgccttcatgcaagtcgcaagatatgtcatcctaagttgtatatgttttctcaaaagatgtatttacttgagaaaatacgtttcaacgttgatggacaaccaattaagaatcaagcacctttcgtaatcaatttaggcttatcgtctaaggtatactgatttacgacagttgactagacttataaggaacaaaataatagaataatttcgtcaagaaaaattgattttggactcatactttacataatattttactcttaaatttatttaagtggctcctcgcatgatcccaaagagcaagcctagactatatggaaaagtttctaaacacttcacctaatcatagagactaagatggtaattcgtaaacgacaaagcctagtgacactgtccattgtaaaattccacatcatattagtattaaacatatacatgtagtttcctgaacacatgtagtatcaaacacacttcgtggcttcttcctcgaaacctggtaccctaggcttaaggtttaaacagcccgggcgcgccgtcccattctggccgaatttaagtgactagggtcacgtgaccctagtcacttaccggattctggccgtaccgagctcgaattcaaaggtcacccggtccgggcctagaaggcctaagtgactagggtcacgtgaccctagtcacttattcccgggcaactttattatacaaagttggcattataaaaaagcattgcttatcaatttgttgcaacgaacaggtcactatcagtcaaaataaaatcattatttggagctccatgcatggtagcgttatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacgg

SEQ ID NO: 60 - DNA, 51162 bp, synthetic construct
  misc_feature 1..51162: note = Vector sequence
  source 1..51162: mol_type = other DNA
tctagagctcgttcctcgaggaacggtacctgcggggaagcttacaataatgtgtgttgttaagtcttgttgcctgtcatcgtctgactgactttcgtcataaatcccggcctccgtaacccagctttgggcaagctcacggatttgatccggcggaacgggaatatcgagatgccgggctgaacgctgcagttccagctttccctttcgggacaggtactccagctgattgattatctgctgaagggtcttggttccacctcctggcacaatgcgaatgattacttgagcgcgatcgggcatccaattttctcccgtcaggtgcgtggtcaagtgctacaaggcacctttcagtaacgagcgaccgtcgatccgtcgccgggatacggacaaaatggagcgcagtagtccatcgagggcggcgaaagcctcgccaaaagcaatacgttcatctcgcacagcctccagatccgatcgagggtcttcggcgtaggcagatagaagcatggatacattgcttgagagtattccgatggactgaagtatggcttccatcttttctcgtgtgtctgcatctatttcgagaaagcccccgatgcggcgcaccgcaacgcgaattgccatactatccgaaagtcccagcaggcgcgcttgataggaaaaggtttcatactcggccgatcgcagacgggcactcacgaccttgaacccttcaactttcagggatcgatgctggttgatggtagtctcactcgacgtggctctggtgtgttttgacatagcttcctccaaagaaagcggaaggtctggatactccagcacgaaatgtgcccgggtagacggatggaagtctagccctgctcaatatgaaatcaacagtacatttacagtcaatactgaatatacttgctacatttgcaattgtcttataacgaatgtgaaataaaaatagtgtaacaacgcttttactcatcgataatcacaaaaacatttatacgaacaaaaatacaaatgcactccggtttcacaggataggcgggatcagaatatgcaacttttgacgttttgttctttcaaagggggtgctggcaaaaccaccgcactcatgggcctttgcgctgctttggcaaatgacggtaaacgagtggccctctttgatgccgacgaaaaccggcctctgacgcgatggagagaaaacgccttacaaagcagtactgggatcctcgctgtgaagtctattccgccgacgaaatgccccttcttgaagcagcctatgaaaatgccgagctcgaaggatttgattatgcgttggccgatacgcgtggcggctcgagcgagctcaacaacacaatcatcgctagctcaaacctgcttctgatccccaccatgctaacgccgctcgacatcgatgaggcactatctacctaccgctacgtcatcgagctgctgttgagtgaaaatttggcaattcctacagctgttttgcgccaacgcgtcccggtcggccgattgacaacatcgcaacgcaggatgtcagagacgctagagagccttccagttgtaccgtctcccatgcatgaaagagatgcatttgccgcgatgaaagaacgcggcatgttgcatcttacattactaaacacgggaactgatccgacgatgcgcctcatagagaggaatcttcggattgcgatggaggaagtcgtggtcatttcgaaactgatcagcaaaatcttggaggcttgaagatggcaattcgcaagcccgcattgtcggtcggcgaagcacggcggcttgctggtgctcgacccgagatccaccatcccaacccgacacttgttccccagaagctggacctccagcacttgcctgaaaaagccgacgagaaagaccagcaacgtgagcctctcgtcgccgatcacatttacagtcccgatcgacaacttaagctaactgtggatgcccttagtccacctccgtccccgaaaaagctccaggtttttctttcagcgcgaccgcccgcgcctcaagtgtcgaaaacatatgacaacctcgttcggcaatacagtccctcgaagtcgctacaaatgattttaaggcgcgcgttggacgatttcgaaagcatgctggcagatggatcatttcgcgtggccccgaaaagttatccgatcccttcaactacagaaaaatccgttctcgttcagacctcacgcatgttcccggttgcgttgctcgaggtcgctcgaagtcattttgatccgttggggttggagaccgctcgagctttcggccacaagctggctaccgccgcgctcgcgtcattctttgctggagagaagccatcgagcaattggtgaagagggacctatcggaacccctcaccaaatattgagtgtaggtttgaggccgctggccgcgtcctcagtcaccttttgagccagataattaagagccaaatgcaattggctcaggctgccatcgtccccccgtgcgaaacctgcacgtccgcgtcaaagaaataaccggcacctcttgctgtttttatcagttgagggcttgacggatccgcctcaagtttgcggcgcagccgcaaaatgagaacatctatactcctgtcgtaaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgcaagaaactctctcgtactaaacggcttagcgataaaatcacttgctcctagctcgagtgcaacaactttatccgtctcctcaaggcggtcgccactgataattatgattggaatatcagactttgccgccagatttcgaacgatctcaagcccatcttcacgacctaaatttagatcaacaaccacgacatcgaccgtcgcggaagagagtactctagtgaactgggtgctgtcggctaccgcggtcactttgaaggcgtggatcgtaaggtattcgataataagatgccgcatagcgacatcgtcatcgataagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacgaaaatgcgaaccaagtatttcaattttatgacaaaagttctcaatcgttgttacaagtgaaacgcttcgaggttacagctactattgattaaggagatcgcctatggtctcgccccggcgtcgtgcgtccgccgcgagccagatctcgcctacttcataaacgtcctcataggcacggaatggaatgatgacatcgatcgccgtagagagcatgtcaatcagtgtgcgatcttccaagctagcaccttgggcgctacttttgacaagggaaaacagtttcttgaatccttggattggattcgcgccgtgtattgttgaaatcgatcccggatgtcccgagacgacttcactcagataagcccatgctgcatcgtcgcgcatctcgccaagcaatatccggtccggccgcatacgcagacttgcttggagcaagtgctcggcgctcacagcacccagcccagcaccgttcttggagtagagtagtctaacatgattatcgtgtggaatgacgagttcgagcgtatcttctatggtgattagcctttcctggggggggatggcgctgatcaaggtcttgctcattgttgtcttgccgcttccggtagggccacatagcaacatcgtcagtcggctgacgacgcatgcgtgcagaaacgcttccaaatccccgttgtcaaaatgctgaaggatagcttcatcatcctgattttggcgtttccttcgtgtctgccactggttccacctcgaagcatcataacgggaggagacttctttaagaccagaaacacgcgagcttggccgtcgaatggtcaagctgacggtgcccgagggaacggtcggcggcagacagatttgtagtcgttcaccaccaggaagttcagtggcgcagagggggttacgtggtccgacatcctgctttctcagcgcgcccgctaaaatagcgatatcttcaagatcatcataagagacgggcaaaggcatcttggtaaaaatgccggcttggcgcacaaatgcctctccaggtcgattgatcgcaatttcttcagtcttcgggtcatcgagccattccaaaatcggcttcagaagaaagcgtagttgcggatccacttccatttacaatgtatcctatctctaagcggaaatttgaattcattaagagcggcggttcctcccccgcgtggcgccgccagtcaggcggagctggtaaacaccaaagaaatcgaggtcccgtgctacgaaaatggaaacggtgtcaccctgattcttcttcagggttggcggtatgttgatggttgccttaagggctgtctcagttgtctgctcaccgttattttgaaagctgttgaagctcatcccgccacccgagctgccggcgtaggtgctagctgcctggaaggcgccttgaacaacactcaagagcatagctccgctaaaacgctgccagaagtggctgtcgaccgagcccggcaatcctgagcgaccgagttcgtccgcgcttggcgatgttaacgagatcatcgcatggtcaggtgtctcggcgcgatcccacaacacaaaaacgcgcccatctccctgttgcaagccacgctgtatttcgccaacaacggtggtgccacgatcaagaagcacgatattgttcgttgttccacgaatatcctgaggcaagacacactttacatagcctgccaaatttgtgtcgattgcggtttgcaagatgcacggaattattgtcccttgcgttaccataaaatcggggtgcggcaagagcgtggcgctgctgggctgcagctcggtgggtttcatacgtatcgacaaatcgttctcgccggacacttcgccattcggcaaggagttgtcgtcacgcttgccttcttgtcttcggcccgtgtcgccctgaatggcgcgtttgctgaccccttgatcgccgctgctatatgcaaaaatcggtgtttcttccggccgtggctcatgccgctccggttcgcccctcggcggtagaggagcagcaggctgaacagcctcttgaaccgctggaggatccggcggcacctcaatcggagctggatgaaatggcttggtgtttgttgcgatcaaagttgacggcgatgcgttctcattcaccttcttttggcgcccacctagccaaatgaggcttaatgataacgcgagaacgacacctccgacgatcaatttctgagaccccgaaagacgccggcgatgtttgtcggagaccagggatccagatgcatcaacctcatgtgccgcttgctgactatcgttattcatcccttcgcccccttcaggacgcgtttcacatcgggcctcaccgtgcccgtttgcggcctttggccaacgggatcgtaagcggtgttccagatacatagtactgtgtggccatccctcagacgccaacctcgggaaaccgaagaaatctcgacatcgctccctttaactgaatagttggcaacagcttccttgccatcaggattgatggtgtagatggagggtatgcgtacattgcccggaaagtggaataccgtcgtaaatccattgtcgaagacttcgagtggcaacagcgaacgatcgccttgggcgacgtagtgccaattactgtccgccgcaccaagggctgtgacaggctgatccaataaattctcagctttccgttgatattgtgcttccgcgtgtagtctgtccacaacagccttctgttgtgcctcccttcgccgagccgccgcatcgtcggcggggtaggcgaattggacgctgtaatagagatcgggctgctctttatcgaggtgggacagagtcttggaacttatactgaaaacataacggcgcatcccggagtcgcttgcggttagcacgattactggctgaggcgtgaggacctggcttgccttgaaaaatagataatttccccgcggtagggctgctagatctttgctatttgaaacggcaaccgctgtcaccgtttcgttcgtggcgaatgttacgaccaaagtagctccaaccgccgtcgagaggcgcaccacttgatcgggattgtaagccaaataacgcatgcgcggatctagcttgcccgccattggagtgtcttcagcctccgcaccagtcgcagcggcaaataaacatgctaaaatgaaaagtgcttttctgatcatggttcgctgtggcctacgtttgaaacggtatcttccgatgtctgataggaggtgacaaccagacctgccgggttggttagtctcaatctgccgggcaagctggtcaccttttcgtagcgaactgtcgcggtccacgtactcaccacaggcattttgccgtcaacgacgagggtccttttatagcgaatttgctgcgtgcttggagttacatcatttgaagcgatgtgctcgacctccaccctgccgcgtttgccaagaatgacttgaggcgaactgggattgggatagttgaagaattgctggtaatcctggcgcactgttggggcactgaagttcgataccaggtcgtaggcgtactgagcggtgtcggcatcataactctcgcgcaggcgaacgtactcccacaatgaggcgttaacgacggcctcctcttgagttgcaggcaatcgcgagacagacacctcgctgtcaacggtgccgtccggccgtatccatagatatacgggcacaagcctgctcaacggcaccattgtggctatagcgaacgcttgagcaacatttcccaaaatcgcgatagctgcgacagctgcaatgagtttggagagacgtcgcgccgatttcgctcgcgcggtttgaaaggcttctacttccttatagtgctcggcaaggctttcgcgcgccactagcatggcatattcaggccccgtcatagcgtccacccgaattgccgagctgaagatctgacggagtaggctgccatcgccccacattcagcgggaagatcgggcctttgcagctcgctaatgtgtcgtttgtctggcagccgctcaaagcgacaactaggcacagcaggcaatacttcatagaattctccattgaggcgaatttttgcgcgacctagcctcgctcaacctgagcgaagcgacggtacaagctgctggcagattgggttgcgccgctccagtaactgcctccaatgttgccggcgatcgccggcaaagcgacaatgagcgcatcccctgtcagaaaaaacatatcgagttcgtaaagaccaatgatcttggccgcggtcgtaccggcgaaggtgattacaccaagcataagggtgagcgcagtcgcttcggttaggatgacgatcgttgccacgaggtttaagaggagaagcaagagaccgtaggtgataagttgcccgatccacttagctgcgatgtcccgcgtgcgatcaaaaatatatccgacgaggatcagaggcccgatcgcgagaagcactttcgtgagaattccaacggcgtcgtaaactccgaaggcagaccagagcgtgccgtaaaggacccactgtgccccttggaaagcaaggatgtcctggtcgttcatcggaccgatttcggatgcgattttctgaaaaacggcctgggtcacggcgaacattgtatccaactgtgccggaacagtctgcagaggcaagccggttacactaaactgctgaacaaagtttgggaccgtcttttcgaagatggaaaccacatagtcttggtagttagcctgcccaacaattagagcaacaacgatggtgaccgtgatcacccgagtgataccgctacgggtatcgacttcgccgcgtatgactaaaataccctgaacaataatccaaagagtgacacaggcgatcaatggcgcactcaccgcctcctggatagtctcaagcatcgagtccaagcctgtcgtgaaggctacatcgaagatcgtatgaatggccgtaaacggcgccggaatcgtgaaattcatcgattggacctgaacttgactggtttgtcgcataatgttggataaaatgagctcgcattcggcgaggatgcgggcggatgaacaaatcgcccagccttaggggagggcaccaaagatgacagcggtcttttgatgctccttgcgttgagcggccgcctcttccgcctcgtgaaggccggcctgcgcggtagtcatcgttaataggcttgtcgcctgtacattttgaatcattgcgtcatggatctgcttgagaagcaaaccattggtcacggttgcctgcatgatattgcgagatcgggaaagctgagcagacgtatcagcattcgccgtcaagcgtttgtccatcgtttccagattgtcagccgcaatgccagcgctgtttgcggaaccggtgatctgcgatcgcaacaggtccgcttcagcatcactacccacgactgcacgatctgtatcgctggtgatcgcacgtgccgtggtcgacattggcattcgcggcgaaaacatttcattgtctaggtccttcgtcgaaggatactgatttttctggttgagcgaagtcagtagtccagtaacgccgtaggccgacgtcaacatcgtaaccatcgctatagtctgagtgagattctccgcagtcgcgagcgcagtcgcgagcgtctcagcctccgttgccgggtcgctaacaacaaactgcgcccgcgcgggctgaatatatagaaagctgcaggtcaaaactgttgcaataagttgcgtcgtcttcatcgtttcctaccttatcaatcttctgcctcgtggtgacgggccatgaattcgctgagccagccagatgagttgccttcttgtgcctcgcgtagtcgagttgcaaagcgcaccgtgttggcacgccccgaaagcacggcgacatattcacgcatatcccgcagatcaaattcgcagatgacgcttccactttctcgtttaagaagaaacttacggctgccgaccgtcatgtcttcacggatcgcctgaaattccttttcggtacatttcagtccatcgacataagccgatcgatctgcggttggtgatggatagaaaatcttcgtcatacattgcgcaaccaagctggctcctagcggcgattccagaacatgctctggttgctgcgttgccagtattagcatcccgttgttttttcgaacggtcaggaggaatttgtcgacgacagtcgaaaatttagggtttaacaaataggcgcgaaactcatcgcagctcatcacaaaacggcggccgtcgatcatggctccaatccgatgcaggagatatgctgcagcgggagcgcatacttcctcgtattcgagaagatgcgtcatgtcgaagccggtaatcgacggatctaactttacttcgtcaacttcgccgtcaaatgcccagccaagcgcatggccccggcaccagcgttggagccgcgctcctgcgccttcggcgggcccatgcaacaaaaattcacgtaaccccgcgattgaacgcatttgtggatcaaacgagagctgacgatggataccacggaccagacggcggttctcttccggagaaatcccaccccgaccatcactctcgatgagagccacgatccattcgcgcagaaaatcgtgtgaggctgctgtgttttctaggccacgcaacggcgccaacccgctgggtgtgcctctgtgaagtgccaaatatgttcctcctgtggcgcgaaccagcaattcgccaccccggtccttgtcaaagaacacgaccgtacctgcacggtcgaccatgctctgttcgagcatggctagaacaaacatcatgagcgtcgtcttacccctcccgataggcccgaatattgccgtcatgccaacatcgtgctcatgcgggatatagtcgaaaggcgttccgccattggtacgaaatcgggcaatcgcgttgccccagtggcctgagctggcgccctctggaaagttttcgaaagagacaaaccctgcgaaattgcgtgaagtgattgcgccagggcgtgtgcgccacttaaaattccccggcaattgggaccaataggccgcttccataccaataccttcttggacaaccacggcacctgcatccgccattcgtgtccgagcccgcgcgcccctgtccccaagactattgagatcgtctgcatagacgcaaaggctcaaatgatgtgagcccataacgaattcgttgctcgcaagtgcgtcctcagcctcggataatttgccgatttgagtcacggctttatcgccggaactcagcatctggctcgatttgaggctaagtttcgcgtgcgcttgcgggcgagtcaggaacgaaaaactctgcgtgagaacaagtggaaaatcgagggatagcagcgcgttgagcatgcccggccgtgtttttgcagggtattcgcgaaacgaatagatggatccaacgtaactgtcttttggcgttctgatctcgagtcctcgcttgccgcaaatgactctgtcggtataaatcgaagcgccgagtgagccgctgacgaccggaaccggtgtgaaccgaccagtcatgatcaaccgtagcgcttcgccaatttcggtgaagagcacaccctgcttctcgcggatgccaagacgatgcaggccatacgctttaagagagccagcgacaacatgccaaagatcttccatgttcctgatctggcccgtgagatcgttttccctttttccgcttagcttggtgaacctcctctttaccttccctaaagccgcctgtgggtagacaatcaacgtaaggaagtgttcattgcggaggagttggccggagagcacgcgctgttcaaaagcttcgttcaggctagcggcgaaaacactacggaagtgtcgcggcgccgatgatggcacgtcggcatgacgtacgaggtgagcatatattgacacatgatcatcagcgatattgcgcaacagcgtgttgaacgcacgacaacgcgcattgcgcatttcagtttcctcaagctcgaatgcaacgccatcaattctcgcaatggtcatgatcgatccgtcttcaagaaggacgatatggtcgctgaggtggccaatataagggagatagatctcaccggatctttcggtcgttccactcgcgccgagcatcacaccattcctctccctcgtgggggaaccctaattggatttgggctaacagtagcgcccccccaaactgcactatcaatgcttcttcccgcggtccgcaaaaatagcaggacgacgctcgccgcattgtagtctcgctccacgatgagccgggctgcaaaccataacggcacgagaacgacttcgtagagcgggttctgaacgataacgatgacaaagccggcgaacatcatgaataaccctgccaatgtcagtggcaccccaagaaacaatgcgggccgtgtggctgcgaggtaaagggtcgattcttccaaacgatcagccatcaactaccgccagtgagcgtttggccgaggaagctcgccccaaacatgataacaatgccgccgacgacgccggcaaccagcccaagcgaagcccgcccgaacatccaggagatcccgatagcgacaatgccgagaacagcgagtgactggccgaacggaccaaggataaacgtgcatatattgttaaccattgtggcggggtcagtgccgccacccgcagattgcgctgcggcgggtccggatgaggaaatgctccatgcaattgcaccgcacaagcttggggcgcagctcgatatcacgcgcatcatcgcattcgagagcgagaggcgatttagatgtaaacggtatctctcaaagcatcgcatcaatgcgcacctccttagtataagtcgaataagacttgattgtcgtctgcggatttgccgttgtcctggtgtggcggtggcggagcgattaaaccgccagcgccatcctcctgcgagcggcgctgatatgacccccaaacatcccacgtctcttcggattttagcgcctcgtgatcgtcttttggaggctcgattaacgcgggcaccagcgattgagcagctgtttcaacttttcgcacgtagccgtttgcaaaaccgccgatgaaattaccggtgttgtaagcggagatcgcccgacgaagcgcaaattgcttctcgtcaatcgtttcgccgcctgcataacgacttttcagcatgtttgcagcggcagataatgatgtgcacgcctggagcgcaccgtcaggtgtcagaccgagcatagaaaaatttcgagagtttatttgcatgaggccaacatccagcgaatgccgtgcatcgagacggtgcctgacgacttgggttgcttggctgtgatcttgccagtgaagcgtttcgccggtcgtgttgtcatgaatcgctaaaggatcaaagcgactctccaccttagctatcgccgcaagcgtagatgtcgcaactgatggggcacacttgcgagcaacatggtcaaactcagcagatgagagtggcgtggcaaggctcgacgaacagaaggagaccatcaaggcaagagaaagcgaccccgatctcttaagcataccttatctccttagctcgcaactaacaccgcctctcccgttggaagaagtgcgttgttttatgttgaagattatcgggagggtcggttactcgaaaattttcaattgcttctttatgatttcaattgaagcgagaaacctcgcccggcgtcttggaacgcaacatggaccgagaaccgcgcatccatgactaagcaaccggatcgacctattcaggccgcagttggtcaggtcaggctcagaacgaaaatgctcggcgaggttacgctgtctgtaaacccattcgatgaacgggaagcttccttccgattgctcttggcaggaatattggcccatgcctgcttgcgctttgcaaatgctcttatcgcgttggtatcatatgccttgtccgccagcagaaacgcactctaagcgattatttgtaaaaatgtttcggtcatgcggcggtcatgggcttgacccgctgtcagcgcaagacggatcggtcaaccgtcggcatcgacaacagcgtgaatcttggtggtcaaaccgccacgggaacgtcccatacagccatcgtcttgatcccgctgtttcccgtcgccgcatgttggtggacgcggacacaggaactgtcaatcatgacgacattctatcgaaagccttggaaatcacactcagaatatgatcccagacgtctgcctcacgccatcgtacaaagcgattgtagcaggttgtacaggaaccgtatcgatcaggaacgtctgcccagggcgggcccgtccggaagcgccacaagatgacattgatcacccgcgtcaacgcgcggcacgcgacgcggcttatttgggaacaaaggactgaacaacagtccattcgaaatcggtgacatcaaagcggggacgggttatcagtggcctccaagtcaagcctcaatgaatcaaaatcagaccgatttgcaaacctgatttatgagtgtgcggcctaaatgatgaaatcgtccttctagatcgcctccgtggtgtagcaacacctcgcagtatcgccgtgctgaccttggccagggaattgactggcaagggtgctttcacatgaccgctcttttggccgcgatagatgatttcgttgctgctttgggcacgtagaaggagagaagtcatatcggagaaattcctcctggcgcgagagcctgctctatcgcgacggcatcccactgtcgggaacagaccggatcattcacgaggcgaaagtcgtcaacacatgcgttataggcatcttcccttgaaggatgatcttgttgctgccaatctggaggtgcggcagccgcaggcagatgcgatctcagcgcaacttgcggcaaaacatctcactcacctgaaaaccactagcgagtctcgcgatcagacgaaggccttttacttaacgacacaatatccgatgtctgcatcacaggcgtcgctatcccagtcaatactaaagcggtgcaggaactaaagattactgatgacttaggcgtgccacgaggcctgagacgacgcgcgtagacagttttttgaaatcattatcaaagtgatggcctccgctgaagcctatcacctctgcgccggtctgtcggagagatgggcaagcattattacggtcttcgcgcccgtacatgcattggacgattgcagggtcaatggatctgagatcatccagaggattgccgcccttaccttccgtttcgagttggagccagcccctaaatgagacgacatagtcgacttgatgtgacaatgccaagagagagatttgcttaacccgatttttttgctcaagcgtaagcctattgaagcttgccggcatgacgtccgcgccgaaagaatatcctacaagtaaaacattctgcacaccgaaatgcttggtgtagacatcgattatgtgaccaagatccttagcagtttcgcttggggaccgctccgaccagaaataccgaagtgaactgacgccaatgacaggaatcccttccgtctgcagataggtaccatcgatagatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccaagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggataagtgactagggtcacgtgaccctagtcacttatcgagctagttaccctatgaggtgacatgaagcgctcacggttactatgacggttagcttcacgactgttggtggcagtagcgtacgacttagctatagttccggtagatctgaagttcctattccgaagttcctattcttcaaaaggtataggaacttcctcgaattgttgtggtggggtatagaggtttgatataggtggaactgctgtagagcgtggagatatagggggaaagagaacgctgatgtgacaagtgagtgagatatagggggagaaatttagggggaacgccgaacacagtctaaagaagcttgggacccaaagcactctgttcgggggtttttttttttgtctttcaactttttgctgtaatgttattcaaaataagaaaagcacttggcatggctaagaaatagagttcaacaactgaacagtacagtgtattatcaatggcataaaaaacaacccttacagcattgccgtattttattgatcaaacattcaactcaacactgacgagtggtcttccaccgatcaacggactaatgctgctttgtcagatcaccggttaagtgactagggtcacgtgaccctagtcacttaggttaccagagctggtcacctttgtccaccaagatggaactgcggccgctcattaattaagtcaggcgcgcctctagttgaagacacgttcatgtcttcatcgtaagaagacactcagtagtcttcggccagaatggccatctggattcagcaggcataacttcgtataatgtatgctatacgaagttatctctagaactagtggatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcccccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccataacttcgtataatgtatgctatacgaagttatctagaaggccatttaaatcctgaggatctggtcttcctaaggacccgggatatcgctatcaactttgtatagaaaagttgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggggactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcggcaaaagagccgttcatttcaataaaccgggcgacctcagccatcccttcctgattttccgctttccagcgttcggcacgcagacgacgggcttcattctgcatggttgtgcttaccgaaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatcctctagattacgccccgcctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctggggtgccctaatgcggccgccatagtgactggatatgttgtgttttacagtattatgtagtctgttttttatgcaaaatctaatttaatatattgatatttatatcattttacgtttctcgttcaactttattatacaaagttgatagatatcggaccgattaaactttaattcggtccgatgcatgtatacgaagttcctattccgaagttcctattctacatagagtataggaacttcacctggtggcgccgctagtggatcccccgggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgctctagcgtatacgaagttcctattccgaagttcctattctctagaaagtataggaacttctgattccgatgacttcgtaggttcctagctcaagccgctcgtgtccaagcgtcacttacgattagctaatgattacggcatctaggaccgactagtaagtgactagggtcacgtgaccctagtcacttatacgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacagtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcggagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccactcttcattaaccgctatatcgaaaattgcttgcggcttgttagaattgccatgacgtacctcggtgtcacgggtaagattaccgataaactggaactgattatggctcatatcgaaagtctccttgagaaaggagactctagtttagctaaacattggttccgctgtcaagaactttagcggctaaaattttgcgggccgcgaccaaaggtgcgaggggcggcttccgctgtgtacaaccagatatttttcaccaacatccttcgtctgctcgatgagcggggcatgacgaaacatgagctgtcggagagggcaggggtttcaatttcgtttttatcagacttaaccaacggtaaggccaacccctcgttgaaggtgatggaggccattgccgacgccctggaaactcccctacctcttctcctggagtccaccgaccttgaccgcgaggcactcgcggagattgcgggtcatcctttcaagagcagcgtgccgcccggatacgaacgcatcagtgtggttttgccgtcacataaggcgtttatcgtaaagaaatggggcgacgacacccgaaaaaagctgcgtggaaggctctgacgccaagggttagggcttgcacttccttctttagccgctaaaacggccccttctctgcgggccgtcggctcgcgcatcatatcgacatcctcaacggaagccgtgccgcgaatggcatcgggcgggtgcgctttgacagttgttttctatcagaacccctacgtcgtgcggttcgattagctgtttgtcttgcaggctaaacactttcggtatatcgtttgcctgtgcgataatgttgctaatgatttgttgcgtaggggttactgaaaagtgagcgggaaagaagagtttcagaccatcaaggagcgggccaagcgcaagctggaacgcgacatgggtgcggacctgttggccgcgctcaacgacccgaaaaccgttgaagtcatgctcaacgcggacggcaaggtgtggcacgaacgccttggcgagccgatgcggtacatctgcgacatgcggcccagccagtcgcaggcgattatagaaacggtggccggattccacggcaaagaggtcacgcggcattcgcccatcctggaaggcgagttccccttggatggcagccgctttgccggccaattgccgccggtcgtggccgcgccaacctttgcgatccgcaagcgcgcggtcgccatcttcacgctggaacagtacgtcgaggcgggcatcatgacccgcgagcaatacgaggtcattaaaagcgccgtcgcggcgcatcgaaacatcctcgtcattggcggtactggctcgggcaagaccacgctcgtcaacgcgatcatcaatgaaatggtcgccttcaacccgtctgagcgcgtcgtcatcatcgaggacaccggcgaaatccagtgcgccgcagagaacgccgtccaataccacaccagcatcgacgtctcgatgacgctgctgctcaagacaacgctgcgtatgcgccccgaccgcatcctggtcggtgaggtacgtggccccgaagcccttgatctgttgatggcctggaacaccgggcatgaaggaggtgccgccaccctgcacgcaaacaaccccaaagcgggcctgagccggctcgccatgcttatcagcatgcacccggattcaccgaaacccattgagccgctgattggcgaggcggttcatgtggtcgtccatatcgccaggacccctagcggccgtcgagtgcaagaaattctcgaagttcttggttacgagaacggccagtacatcaccaaaaccctgtaaggagtatttccaatgacaacggctgttccgttccgtctgaccatgaatcgcggcattttgttctaccttgccgtgttcttcgttctcgctctcgcgttatccgcgcatccggcgatggcctcggaaggcaccggcggcagcttgccatatgagagctggctgacgaacctgcgcaactccgtaaccggcccggtggccttcgcgctgtccatcatcggcatcgtcgtcgccggcggcgtgctgatcttcggcggcgaactcaacgccttcttccgaaccctgatcttcctggttctggtgatggcgctgctggtcggcgcgcagaacgtgatgagcaccttcttcggtcgtggtgccgaaatcgcggccctcggcaacggggcgctgcaccaggtgcaagtcgcggcggcggatgccgtgcgtgcggtagcggctggacggctcgcctaatcatggctctgcgcacgatccccatccgtcgcgcaggcaaccgagaaaacctgttcatgggtggtgatcgtgaactggtgatgttctcgggcctgatggcgtttgcgctgattttcagcgcccaagagctgcgggccaccgtggtcggtctgatcctgtggttcggggcgctctatgcgttccgaatcatggcgaaggccgatccgaagatgcggttcgtgtacctgcgtcaccgccggtacaagccgtattacccggcccgctcgaccccgttccgcgagaacaccaatagccaagggaagcaataccgatgatccaagcaattgcgattgcaatcgcgggcctcggcgcgcttctgttgttcatcctctttgcccgcatccgcgcggtcgatgccgaactgaaactgaaaaagcatcgttccaaggacgccggcctggccgatctgctcaactacgccgctgtcgtcgatgacggcgtaatcgtgggcaagaacggcagctttatggctgcctggctgtacaagggcgatgacaacgcaagcagcaccgaccagcagcgcgaagtagtgtccgcccgcatcaaccaggccctcgcgggcctgggaagtgggtggatgatccatgtggacgccgtgcggcgtcctgctccgaactacgcggagcggggcctgtcggcgttccctgaccgtctgacggcagcgattgaagaagagcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatctactgtaatggcgaaaattcacatggttttgcagggcaagggcggggtcggcaagtcggccatcgccgcgatcattgcgcagtacaagatggacaaggggcagacacccttgtgcatcgacaccgacccggtgaacgcgacgttcgagggctacaaggccctgaacgtccgccggctgaacatcatggccggcgacgaaattaactcgcgcaacttcgacaccctggtcgagctgattgcgccgaccaaggatgacgtggtgatcgacaacggtgccagctcgttcgtgcctctgtcgcattacctcatcagcaaccaggtgccggctctgctgcaagaaatggggcatgagctggtcatccataccgtcgtcaccggcggccaggctctcctggacacggtgagcggcttcgcccagctcgccagccagttcccggccgaagcgcttttcgtggtctggctgaacccgtattgggggcctatcgagcatgagggcaagagctttgagcagatgaaggcgtacacggccaacaaggcccgcgtgtcgtccatcatccagattccggccctcaaggaagaaacctacggccgcgatttcagcgacatgctgcaagagcggctgacgttcgaccaggcgctggccgatgaatcgctcacgatcatgacgcggcaacgcctcaagatcgtgcggcgcggcctgtttgaacagctcgacgcggcggccgtgctatgagcgaccagattgaagagctgatccgggagattgcggccaagcacggcatcgccgtcggccgcgacgacccggtgctgatcctgcataccatcaacgcccggctcatggccgacagtgcggccaagcaagaggaaatccttgccgcgttcaaggaagagctggaagggatcgcccatcgttggggcgaggacgccaaggccaaagcggagcggatgctgaacgcggccctggcggccagcaaggacgcaatggcgaaggtaatgaaggacagcgccgcgcaggcggccgaagcgatccgcagggaaatcgacgacggccttggccgccagctcgcggccaaggtcgcggacgcgcggcgcgtggcgatgatgaacatgatcgccggcggcatggtgttgttcgcggccgccctggtggtgtgggcctcgttatgaatcgcagaggcgcagatgaaaaagcccggcgttgccgggctttgtttttgcgttagctgggcttgtttgacaggcccaagctctgactgcgcccgcgctcgcgctcctgggcctgtttcttctcctgctcctgcttgcgcatcagggcctggtgccgtcgggctgcttcacgcatcgaatcccagtcgccggccagctcgggatgctccgcgcgcatcttgcgcgtcgccagttcctcgatcttgggcgcgtgaatgcccatgccttccttgatttcgcgcaccatgtccagccgcgtgtgcagggtctgcaagcgggcttgctgttgggcctgctgctgctgccaggcggcctttgtacgcggcagggacagcaagccgggggcattggactgtagctgctgcaaacgcgcctgctgacggtctacgagctgttctaggcggtcctcgatgcgctccacctggtcatgctttgcctgcacgtagagcgcaagggtctgctggtaggtctgctcgatgggcgcggattctaagagggcctgctgttccgtctcggcctcctgggccgcctgtagcaaatcctcgccgctgttgccgctggactgctttactgccggggactgctgttgccctgctcgcgccgtcgtcgcagttcggcttgcccccactcgattgactgcttcatttcgagccgcagcgatgcgatctcggattgcgtcaacggacggggcagcgcggaggtgtccggcttctccttgggtgagtcggtcgatgccatagccaaaggtttccttccaaaatgcgtccattgctggaccgtgtttctcattgatgcccgcaagcatcttcggcttgaccgccaggtcaagcgcgccttcatgggcggtcatgacggacgccgccatgaccttgccgccgttgttctcgatgtagccgcgtaatgaggcaatggtgccgcccatcgtcagcgtgtcatcgacaacgatgtacttctggccggggatcacctccccctcgaaagtcgggttgaacgccaggcgatgatctgaaccggctccggttcgggcgaccttctcccgctgcacaatgtccgtttcgacctcaaggccaaggcggtcggccagaacgaccgccatcatggccggaatcttgttgttccccgccgcctcgacggcgaggactggaacgatgcggggcttgtcgtcgccgatcagcgtcttgagctgggcaacagtgtcgtccgaaatcaggcgctcgaccaaattaagcgccgcttccgcgtcgccctgcttcgcagcctggtattcaggctcgttggtcaaagaaccaaggtcgccgttgcgaaccaccttcgggaagtctccccacggtgcgcgctcggctctgctgtagctgctcaagacgcctccctttttagccgctaaaactctaacgagtgcgcccgcgactcaacttgacgctttcggcacttacctgtgccttgccacttgcgtcataggtgatgcttttcgcactcccgatttcaggtactttatcgaaatctgaccgggcgtgcattacaaagttcttccccacctgttggtaaatgctgccgctatctgcgtggacgatgctgccgtcgtggcgctgcgacttatcggccttttgggccatatagatgttgtaaatgccaggtttcagggccccggctttatctaccttctggttcgtccatgcgccttggttctcggtctggacaattctttgcccattcatgaccaggaggcggtgtttcattgggtgactcctgacggttgcctctggtgttaaacgtgtcctggtcgcttgccggctaaaaaaaagccgacctcggcagttcgaggccggctttccctagagccgggcgcgtcaaggttgttccatctattttagtgaactgcgttcgatttatcagttactttcctcccgctttgtgtttcctcccactcgtttccgcgtctagccgacccctcaacatagcggcctcttcttgggctgcctttgcctcttgccgcgcttcgtcacgctcggcttgcaccgtcgtaaagcgctcggcctgcctggccgcctcttgcgccgccaacttcctttgctcctggtgggcctcggcgtcggcctgcgccttcgctttcaccgctgccaactccgtgcgcaaactctccgcttcgcgcctggtggcgtcgcgctcgccgcgaagcgcctgcatttcctggttggccgcgtccagggtcttgcggctctcttctttgaatgcgcgggcgtcctggtgagcgtagtccagctcggcgcgcagctcctgcgctcgacgctccacctcgtcggcccgctgcgtcgccagcgcggcccgctgctcggctcctgccagggcggtgcgtgcttcggccagggcttgccgctggcgtgcggccagctcggccgcctcggcggcctgctgctctagcaatgtaacgcgcgcctgggcttcttccagctcgcgggcctgcgcctcgaaggcgtcggccagctccccgcgcacggcttccaactcgttgcgctcacgatcccagccggcttgcgctgcctgcaacgattcattggcaagggcctgggcggcttgccagagggcggccacggcctggttgccggcctgctgcaccgcgtccggcacctggactgccagcggggcggcctgcgccgtgcgctggcgtcgccattcgcgcatgccggcgctggcgtcgttcatgttgacgcgggcggccttacgcactgcatccacggtcgggaagttctcccggtcgccttgctcgaacagctcgtccgcagccgcaaaaatgcggtcgcgcgtctctttgttcagttccatgttggctccggtaattggtaagaataataatactcttacctaccttatcagcgcaagagtttagctgaacagttctcgacttaacggcaggttttttagcggctgaagggcaggcaaaaaaagccccgcacggtcggcgggggcaaagggtcagcgggaaggggattagcgggcgtcgggcttcttcatgcgtcggggccgcgcttcttgggatggagcacgacgaagcgcgcacgcgcatcgtcctcggccctatcggcccgcgtcgcggtcaggaacttgtcgcgcgctaggtcctccctggtgggcaccaggggcatgaactcggcctgctcgatgtaggtccactccatgaccgcatcgcagtcgaggccgcgttccttcaccgtctcttgcaggtcgcggtacgcccgctcgttgagcggctggtaacgggccaattggtcgtaaatggctgtcggccatgagcggcctttcctgttgagccagcagccgacgacgaagccggcaatgcaggcccctggcacaaccaggccgacgccgggggcaggggatggcagcagctcgccaaccaggaaccccgccgcgatgatgccgatgccggtcaaccagcccttgaaactatccggccccgaaacacccctgcgcattgcctggatgctgcgccggatagcttgcaacatcaggagccgtttcttttgttcgtcagtcatggtccgccctcaccagttgttcgtatcggtgtcggacgaactgaaatcgcaagagctgccggtatcggtccagccgctgtccgtgtcgctgctgccgaagcacggcgaggggtccgcgaacgccgcagacggcgtatccggccgcagcgcatcgcccagcatggccccggtcagcgagccgccggccaggtagcccagcatggtgctgttggtcgccccggccaccagggccgacgtgacgaaatcgccgtcattccctctggattgttcgctgctcggcggggcagtgcgccgcgccggcggcgtcgtggatggctcgggttggctggcctgcgacggccggcgaaaggtgcgcagcagctcgttatcgaccggctgcggcgtcggggccgccgccttgcgctgcggtcggtgttccttcttcggctcgcgcagcttgaacagcatgatcgcggaaaccagcagcaacgccgcgcctacgcctcccgcgatgtagaacagcatcggattcattcttcggtcctccttgtagcggaaccgttgtctgtgcggcgcgggtggcccgcgccgctgtctttggggatcagccctcgatgagcgcgaccagtttcacgtcggcaaggttcgcctcgaactcctggccgtcgtcctcgtacttcaaccaggcatagccttccgccggcggccgacggttgaggataaggcgggcagggcgctcgtcgtgctcgacctggacgatggcctttttcagcttgtccgggtccggctccttcgcgcccttttccttggcgtccttaccgtcctggtcgccgtcctcgccgtcctggccgtcgccggcctccgcgtcacgctcggcatcagtctggccgttgaaggcatcgacggtgttgggatcgcggcccttctcgtccaggaactcgcgcagcagcttgaccgtgccgcgcgtgatttcctgggtgtcgtcgtcaagccacgcctcgacttcctccgggcgcttcttgaaggccgtcaccagctcgttcaccacggtcacgtcgcgcacgcggccggtgttgaacgcatcggcgatcttctccggcaggtccagcagcgtgacgtgctgggtgatgaacgccggcgacttgccgatttccttggcgatatcgcctttcttcttgcccttcgccagctcgcggccaatgaagtcggcaatttcgcgcggggtcagctcgttgcgttgcaggttctcgataacctggtcggcttcgttgtagtcgttgtcgatgaacgccgggatggacttcttgccggcccacttcgagccacggtagcggcgggcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgctgaattgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtcaatgaaagtttccaacgcgagccattcgcgagagccttgagtccacgctagatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgac

SEQ ID NO: 61 - DNA, 42 bp, synthetic construct
  misc_feature 1..42: note = sense primer for i1
  source 1..42: mol_type = other DNA
ccatgcatacatccaacgccattcgcttacacctgatatccc

SEQ ID NO: 62 - DNA, 46 bp, synthetic construct
  misc_feature 1..46: note = i1-sense oligonucleotide 2
  source 1..46: mol_type = other DNA
ggactggcgccaggtccgttcccgtcccagatccgtccatggcttc

SEQ ID NO: 63 - DNA, 46 bp, synthetic construct
  misc_feature 1..46: note = i1-sense oligonucleotide 3
  source 1..46: mol_type = other DNA
gtccagatctgacctgtcctgacacaccctcacccggatctgtccc

SEQ ID NO: 64 - DNA, 45 bp, synthetic construct
  misc_feature 1..45: note = i1-sense oligonucleotide 4
  source 1..45: mol_type = other DNA
tccttcccctctcccctgcagctggcgccttgggatccattcctg

SEQ ID NO: 65 - DNA, 42 bp, synthetic construct
  misc_feature 1..42: note = i1-antisense oligonucleotide 1
  source 1..42: mol_type = other DNA
ggacctggcgccagtccgggatatcaggtgtaagcgaatggc

SEQ ID NO: 66 - DNA, 49 bp, synthetic construct
  misc_feature 1..49: note = i1-antisenseoligonucleotide 2
  source 1..49: mol_type = other DNA
aggacaggtcagatctggacgaagccatggacggatctgggacgggaac

SEQ ID NO: 67 - DNA, 51 bp, synthetic construct
  misc_feature 1..51: note = i1-antisense oligonucleotide 3
  source 1..51: mol_type = other DNA
ccagctgcaggggagaggggaaggagggacagatccgggtgagggtgtgtc

SEQ ID NO: 68 - DNA, 44 bp, synthetic construct
  misc_feature 1..44: note = i1-antisense oligonucleotide 4
  source 1..44: mol_type = other DNA
ggtggaggcgttgtaagctggaatcaggaatggatcccaaggcg

SEQ ID NO: 69 - DNA, 48 bp, synthetic construct
  misc_feature 1..48: note = i2_flanking sense primer
  source 1..48: mol_type = other DNA
ccatcagatatcccggactggcgccaggtctgcttcgtcctcgctagg

SEQ ID NO: 70 - DNA, 56 bp, synthetic construct
  misc_feature 1..56: note = i2_sense oligonucleotide 2
  source 1..56: mol_type = other DNA
tttcatttcgcggtctgtttgtgccgttggggctagatccgggtcgtggttcaaca

SEQ ID NO: 71 - DNA, 44 bp, synthetic construct
  misc_feature 1..44: note = i2_sense oligonucleotide 3
  source 1..44: mol_type = other DNA
gatctgcttcgttttggtacagatctgcgttcgctcgaatcgag

SEQ ID NO: 72 - DNA, 43 bp, synthetic construct
  misc_feature 1..43: note = i2_sense oligonucleotide 4
  source 1..43: mol_type = other DNA
catgacgttttcatgtgattatgcagctggcgccttgggatcc

SEQ ID NO: 73 - DNA, 52 bp, synthetic construct
  misc_feature 1..52: note = i2_antisense oligonucleotide 1
  source 1..52: mol_type = other DNA
ggcacaaacagaccgcgaaatgaaacctagcgaggacgaagcagacctggcg

SEQ ID NO: 74 - DNA, 53 bp, synthetic construct
  misc_feature 1..53: note = i2_antisense oligonucleotide 2
  source 1..53: mol_type = other DNA
ctgtaccaaaacgaagcagatctgttgaaccacgacccggatctagccccaac

SEQ ID NO: 75 - DNA, 45 bp, synthetic construct
  misc_feature 1..45: note = i2_antisense oligonucleotide 3
  source 1..45: mol_type = other DNA
cataatcacatgaaaacgtcatgctcgattcgagcgaacgcagat

SEQ ID NO: 76 - DNA, 41 bp, synthetic construct
  misc_feature 1..41: note = i2_antisense flanking primer
  source 1..41: mol_type = other DNA
gtggtaagcgaatgcaggaatggatcccaaggcgccagctg

SEQ ID NO: 77 - DNA, 37 bp, synthetic construct
  misc_feature 1..37: note = i3_sense flanking primer
  source 1..37: mol_type = other DNA
ccatcagtactcgatatcccggactggcgccaggtgc

SEQ ID NO: 78 - DNA, 45 bp, synthetic construct
  misc_feature 1..45: note = i3_sense oligonucleotide 2
  source 1..45: mol_type = other DNA
gtgcatgcgcacgctctgcttctgcctccctttcccttttcctcc

SEQ ID NO: 79 - DNA, 50 bp, synthetic construct
  misc_feature 1..50: note = i3_sense oligonucleotide 3
  source 1..50: mol_type = other DNA
gaaagaactgaaacggaacgcatcttcgctcagctggcgccttgggatcc

SEQ ID NO: 80 - DNA, 39 bp, synthetic construct
  misc_feature 1..39: note = i3_antisense oligonucleotide 1
  source 1..39: mol_type = other DNA
tgcgcatgcacgcacctggcgccagtccgggatatcgag

SEQ ID NO: 81 - DNA, 54 bp, synthetic construct
  misc_feature 1..54: note = i3_antisense oligo2
  source 1..54: mol_type = other DNA
cgttccgtttcagttctttcggaggaaaagggaaagggaggcagaagcagagcg

SEQ ID NO: 82 - DNA, 43 bp, synthetic construct
  misc_feature 1..43: note = i3_antisense flanking primer
  source 1..43: mol_type = other DNA
gtcgaggtgatgtggatcccaaggcgccagctgagcgaagatg

SEQ ID NO: 83 - DNA, 50 bp, synthetic construct
  misc_feature 1..50: note = i4_sense flanking primer
  source 1..50: mol_type = other DNA
ccatcagatatcccggactggcgccaggtgcgtcactgtccaggtgcttg

SEQ ID NO: 84 - DNA, 50 bp, synthetic construct
  misc_feature 1..50: note = i4_s2_sense oligo 3
  source 1..50: mol_type = other DNA
gcttggatcagaatattgttggcggtgacactgtcttctctcgatcgatc

SEQ ID NO: 85 - DNA, 45 bp, synthetic construct
  misc_feature 1..45: note = i4_sense oligonucleotide 3
  source 1..45: mol_type = other DNA
gatcgatgacagctggcgccttgggatccacatcaatcaccatgc

SEQ ID NO: 86 - DNA, 49 bp, synthetic construct
  misc_feature 1..49: note = i4_antisense oligonucleotide 1
  source 1..49: mol_type = other DNA
caatattctgatccaagccaagcacctggacagtgacgcacctggcgcc

SEQ ID NO: 87 - DNA, 50 bp, synthetic construct
  misc_feature 1..50: note = i4_antisense oligonucleotide 2
  source 1..50: mol_type = other DNA
cgccagctgtcatcgatcgatcgatcgagagaagacagtgtcaccgccaa

SEQ ID NO: 88 - DNA, 43 bp, synthetic construct
  misc_feature 1..43: note = i4_antisense flanking primer
  source 1..43: mol_type = other DNA
gctggaacgatggaatgcatggtgattgatgtggatcccaagg

SEQ ID NO: 89 - DNA, 48 bp, synthetic construct
  misc_feature 1..48: note = i5_flanking sense primer
  source 1..48: mol_type = other DNA
ccatcagatatcccggactggcgccaggtcggtttccaatctgttgac

SEQ ID NO: 90 - DNA, 47 bp, synthetic construct
  misc_feature 1..47: note = i5_sense oligonucleotide 2
  source 1..47: mol_type = other DNA
catggatccacagatcggagcagttctttcatagtactcagcgatct

SEQ ID NO: 91 - DNA, 50 bp, synthetic construct
  misc_feature 1..50: note = i5_sense oligonucleotide 3
  source 1..50: mol_type = other DNA
gtttgggtcctaaatttcctttccccggctgttgtttagctggcgccttg

SEQ ID NO: 92 - DNA, 50 bp, synthetic construct
  misc_feature 1..50: note = i5_antisense oligonucleotide 1
  source 1..50: mol_type = other DNA
ctccgatctgtggatccatggtcaacagattggaaaccgacctggcgcca

SEQ ID NO: 93 - DNA, 45 bp, synthetic construct
  misc_feature 1..45: note = i5_antisense oligonucleotide 2
  source 1..45: mol_type = other DNA
gaaatttaggacccaaacagatcgctgagtactatgaaagaactg

SEQ ID NO: 94 - DNA, 44 bp, synthetic construct
  misc_feature 1..44: note = i5_antisense flanking primer
  source 1..44: mol_type = other DNA
cgatgtagatctcaaggcgccagctaaacaacagccggggaaag

SEQ ID NO: 95 - DNA, 535 bp, Zea mays
  source 1..535: mol_type = genomic DNA
gtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcag

SEQ ID NO: 96 - DNA, 344 bp, Zea mays
  source 1..344: mol_type = genomic DNA
gtacagtacacacacatatgtatatatgtatgatgtatcccttcgatcgaaggcatgccttggtcgaataactgagtagtcattttattacgttattttgacaagtcagtagttcatccatttgtcccattttttcagctaggaagtttggttacactggccttggtctaataactgagtagtcattttattacgttgtttcgacaagtcagtagctcatccatctgtcccatttttttcagctaggaagtttggttacactggacttggtctaataactgagtagtcattttattacgttgtttcgacaagtcattagctcatccatctgtcccatttttcag

SEQ ID NO: 97 - DNA, 1014 bp, Zea mays
  source 1..1014: mol_type = genomic DNA
gtatgcttgctctcctgttcatctccgtgctaaacctctgtcctctgggtgggtttttgctgggattttgagctaatctgctggccgcggtagaaaagaccgtgtcccctgatgagctcaagcgctcgccttagccgcgtccttgtcccccgccatttcttgcggtttcgctgtgttcccgtgactcgccgggtgcgtcatcgcctgaatcttgtctgggctctgctgacatgttcttggctagttgggtttatagattcctctgatctaaaaccgtgcctgtgctgcgcacagaactctcccctgtcctttcctggggttttggttacgtggtggtagtaagcttggatttgcacatggataaagttgttctaagctccgtggtttgcttgagatcttgctgttattgcgtgccgtgctcacttcttttgcaatccgaggaatgaatttgtcgtttactcgttttggtggattattagcgcgaaaaaaaactctttttttttgttcttttactacgaaaagcatcttcttggattttgctatcttcttttactacgaaaaactcttgagtctaggaatttgaatttgtgatgtccattcttgcagtgcgctgtgctttattgggaagccaaatcctattattttctgcctctagggtctgaatggaatcagtactattgagacaaaatcaatccaatcaagttgatttctttctttaaaaatattatcacagaactaagtgcttgtgcggaatcagtactggcttttgtttggtggaggatcaatacttgcttttgttttggggtggcaactgttttgctataagattccatgtgttcctgttgagatgaatcatatatagtatagctgcatactacaaatctgtttttcaaatttaggttgctttggcatgatcaattttttttcagacagtctttctaagtggtagctcttgatttcttgttcttctacaactggtgctgctgaatcttgaccgtatagctcgaattgcag

SEQ ID NO: 98 - DNA, 1010 bp, Zea mays
  source 1..1010: mol_type = genomic DNA
gtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 99-100 - skipped (2 entries)

SEQ ID NO: 101 - DNA, 867 bp, Zea mays
  source 1..867: mol_type = genomic DNA
gtatgggtgacgtggtggcactcgatctgctgggttcagatctgattttcttggtgacgggatggcttgccggcgatggtgcaggccgtccactccaacgacccctccgggcagcttgaggccaccacacagttcaggaagctgctttctatagttgagattgggatcttatgtgcagttagcattccagatggatagagtttaggggttgagatttgggccatgctcgaggtattaggccatacccaaacgtgagggtatggtcagttgtagctgtttcgggcaattgttgtatacaggacttgacttgtggattgtgagctatcaaaattagtcgttgcaccctctcattttcagattacttaatttactgtctcgtcagaaaaaaaacaaaccctatcctatggcctctgcaacatgcatatgaccatgtatgcccaaaagttctgaaaaaagttatactcctgaaagcatttgatttcatgaaccactattctattttttccagtagtgttctgcttgcagctggggcaattatcattgcatccatgtgtgtgtttgcgcatgcatgtgtgtgttatacattgttttattgtcttaaaatggtatatgcagactatgtgtgtgtttgcgcattagtgtgtgttatacattgttttattgtcttaaaaaggtcatggagagtctgcgtacctcttttcatattcttgtacatcgtagatagcagctttgatatcttgtgggattcttgtatttgttggtgaatccagatgagtgcatggtacttctttcttaattcccactacaactttatgtgaaagttaagtagtaacttgctgattgagtttccataaatttctctcgtag

SEQ ID NO: 102 - DNA, 847 bp, Zea mays
  source 1..847: mol_type = genomic DNA
gtgcgaccggctcgtattctcttccttgaaaagcatctccatcaccatcttcgattgttttctgatctgtcgtcgaggagtgctgcaatttgcagttgcagggcgttagtactcgaatcggggtgaagtactgaagtggcttaggttagggtttttttttcgtcagatctgttgctagtactactaggatctgaaatttcctgcacgatttaagctgccaccatcctgtgttttaggtgtatcgtggatttcgtttgttaatactttgcggcagaaataacaggatgttccgatcgaatttggcatctcgtatttgttgatggtcacgccaattcttgacagattgcgatgtcgtaataagtcatctgccgttccgtgaccggtttggatctggtttgtgtgtggatgaactgcgctatctttgtttctgttattgtcgactaggaattgattgaattcgccttttactatttcgtgaatcaagctctgattctgtaacttttactcatgttgttttcatttcttcggcctgatccaaaattttccagtggaacgatgctttcttttgtgctgtacaactgcaatatttcgtgactcaagctctgattctgtaaaatttacccatgttgttcccatttctcagatctgatgcaggaatgacgctttagttctagtttgtgctgtgcaactgtaatacgactgtactgaaatttctctctcctatgaatttgagatgctcgcctgccttctgattcaaatacttattacactaatggcacctcgcaatcatgttcctttgatcgttttatgatctgaaccacattaaacacctttctatttcacgcag

SEQ ID NO: 103 - DNA, 1448 bp, Zea mays
  source 1..1448: mol_type = genomic DNA
gtgaggccgcccgccgggggttcctcagatctggggccgatgctgggtccgcctagatccacgcgtttctcggctgctccggcgaggattatgtttttttagtgtccgtcgtttgttaataggatatgcacacgtttctctaagagtgggtgagatctttgcggggggtagaaggtccggagttttgctacccgttcgtttatttagtgggtttcacgctgatctggtcatctggcggtgaagttcctattattaggctgcggatgcctgggtgagctcgaatgccattctatttttaccctcttccgggaccgtagcaactgtctaggtagcacaaaatcatcattttgttaggaccgaagcgagtttcaggttgcatgattttttcggaatggaagacaaatatctatgtctgtgtggggtgggtgggtgggggctcggtacctctggggactaacaattggtgtatcccttccccccttcaaacccttcaaactatgtacttttttatgatatatttttgttcactactggcaccgtcccaactgtggatttttttcaagggtggctcatgtgatgtgctactgttgttttgctagtattcgattcacgggccccaagtagggctggcaaacaattcctttttcccacctcttggacgtgtgatgagtgtgggctttggctttggatgcttgtagtagcttagctggtggatctaggctttattgaagttgtcttttaatgcctatccgtgttcttgccctgtgtcggtgctgcagtaaaaggtgtagctttcagttgacatggcccgatctcgcctggcgcctttcttctagactaaaactcggtaatggaaggtgcgtgacggatctgtcacaaattgattatgcaaacacgtagcaagttaggcagagcaccatttggtaagtagatctggacctggaagttcctgggtgggggttctctgccatctgtcacccacgtgtcgtttgttcactgacctttgaacttcccatttagatcagcatggatggccctagtgctcatcagatacttgtccgatatgtggggcttttgttttaagcgggtcaccaatctttgacccgatgtgcatgtgctattttctatgattgagacggcgacacaggaagatgagcacttttggagttacctggttgggggtccatgagttagctgcttacctgtgtggttgacaaatggatgttggatgcttcagtctattaatcacccgttatttattttctgatactgcaaatataaactggacaggttttgacattctttctgaatctactattgtaccattgtattgtacacagtttgaatttgcagcacaatataaccttgtgtggttttatatttacatcctagtgatcctaaccatatgattgacatctaaaaaaaaactcttgctcttttatggacag

SEQ ID NO: 104 - DNA, 169 bp, Zea mays
  source 1..169: mol_type = genomic DNA
gtccgtgccttttttcttttcctttcaatttcacccgcaaaaggttcattctttcatcgatctgggtggttttgaccgggttctatggccctgttcgtccagatctggtgatttcttggctgttctttccatggggttttgacaaaaaaaaaatcacttgtgatggcag

SEQ ID NO: 105 - DNA, 1500 bp, Zea mays
  source 1..1500: mol_type = genomic DNA
atctttttgtgtggagtgacctgttttcaagagctccattgtagagtgacctgttttgccaattttctctcggctatgtgaaccatcaaatatggtaactaacaatatataaaatataagataaattgttgctatggtacatcagatttgtgtggcattgtccatcgcatatgaaactttctatgttaggctgcattccattataaagtcatgtctgttttttacataggccgataaatatatttttcatatctgtatcctaaaaggttttaggcttagtaggtctaaaacagagtatatgaagtgtgatttcagtgccatggggtatgaggatggcgatgttagtcttgatgggcaagtggtacccaagaaagacacttttcgttacttaggatcaatgcttcaaaaggagggagacatcgaggaggatgtcagtcatagaattaaagtcggatggttgaagtggcgacaagctgcgggtgtcttatgcgaccaccgggtgccacgcaaactaaaaggcaaattctacaggacagcaatccggccggctatgttgtatggagcagaatgttggcccactaaaagacgacatgtccaacaactaagtgtggcagagatgcgtatgttgcgctggatatgtggccacacaaggagagatcgagtccggaatgatgatatacgagagagagtaggagtggcgccaattgaggagaagcttatgcaacatcgcttgagatggtttggacatatccaacgaagacctgaagaggcaccagtgcatatcggaataattaggcgtcccgaaaatgtgaagagaggtagaggtcgaccaactttgacgtggacagaggctgtgaagagagacctgaaggagtggaataatgacaaagagctcgccgcagataggaaggggtggaagtgtgcaattcacgtgccagaaccctgattgatagtttcgcttttcctccttaatcgtttgaccttttcttgtgtccattttagatcttgctggtccttgtgggttttatctcttttatgtgtttccccgtttcgttgttttcggttctcctttgcctttgtttcccttttctgttctttgggggttgagctctgaggttttcatacggggtttcatctctagcctaccccaacgtgcttgggacaaaaaggctttgttgttgttgttgttgttgtatctgtatcctaaaaggtgagagagaagggttattaagaaaaaccctcgtcgctggccactgaaggccgggcccaatttagaacctagacctgctgccaccgcactacaagaccgaggcctaaaaggcccatcaggaggcgcatcggcgaatgccccaaactaaaaccctaccccggcaagtatatatatcctcccaacctcagttcttgttcccattatcacggcggcggtggcggagcgtaaggcgaaggagtagcagcagcaggcggcgccgagtagcggctccccatctcgagcttgccacc

SEQ ID NO: 106 - DNA, 1500 bp, Zea mays
  source 1..1500: mol_type = genomic DNA
ataagcgctactacttgagaaatgattttacatttcacacaaaatgatggtttttatgcaatcaaactagcacctaaatgtaaacaaaacatgcttgtcatgtatctcctttaagcgctattttgagaactctgtttttacaatgaatttatattttctcacataaatttaattcattttttcttagaaaaaaaaaagaaaatccttgagaaaacagagttcccaaactagccataaagttgcacggagttttcttctagcgagattacatcaattgtttgaggtacaagtatttcgtatatgccaaattattagcaccctagttatttagattcttaaatatgttttggggtaaaaatataacataccatgttatacccaacttttgtcaaagatttaggagagtttttttaatcgaacttgatgtttagcgcctaagaattttattggtacttgtaaaaaaaatgttaacatgcccccattagaatgtaggaaaaaatgggagaaaaaactatgatttcaatccctatgaattgatttgttctatagcctttgttttgctagaatttgtctgaaaacgtaagagtgggttcgttttcacacgaaaagatctactcaaatatcattattccttgtttacacccatagttcattcaactacctattttatgttaattttttctgttttcactcctccagatatctattcttcttaacttttttgtttttcaccccataactttgcatagatatttctatcttatttatgtttttttctattgttgactttttcaatatgacattcaaaagaaggttgctgatatttttcccaatcttgcatttaaagaggaccatacttaaaaagactctagaagctttgagagcatctctaacaataccttaaactagtgtctcaaattaaaatacaaggctgtacgcagaaaaaactactccaacaatgcttcattttataaaatttggtcaaaatttttttagtgcactctcttatatgtctcaaatatactacaccacaatgttctgccctataatctagatttggggttttactattggagcagaatattttattggtgctctaaatcaaataaaatataaccattttaaaattatagggcatttttatagatcacgtgttgttggagatgctctaacaaaaccatttaaaagatgaacgttatcaatgtaataatattttgatctgtagtgtttataaatatatatacaataatttttaaaaacttataataatatcattattgtatcatcaatctataaacaaatttaagttttcattaaaaatagtaagtagtctgacattgacttttttttcattcgagaaggacaaactggaaaagaaatatagcccaggggtagttttgggaaaaaagatatttggatgctggcgaatgtgacggccacgtctagtggaagcgcgtataaatcgtcccttctttctttcctcacccgcccctgatccac

SEQ ID NO: 107 - DNA, 1500 bp, Zea mays
  source 1..1500: mol_type = genomic DNA
cgagcggatgttgttccggcccgccaagcggcaatcagcagctcaggtagtgtctagccagtgacgcggggcgcgctactgtatgtgtctcgccgcctcgctgggggcacttcgtgtcggtagtacaggaaaaagggaaggagtcgagatcgttcgcttcactgtctgtgtttcgtgtcggtaggtaccgtatagtgggggagaagagatcgttcgcatcagtgcttgtgtttcacttagttgaacgtatacggtatatcgtatttggcccattgcaaagcaaggtgccggaccaagacaccatccatccatttgttgtagtagcaaaacaacagattcatctgtttggaagtcctattttgagccatacgttctcagtcccgttgttgggacgtgggagaatcctaatcccaaaaatctgcaaccaagtcggggggctagtgaaggaggagaaatttactattttgccactctcaattttggctgtcttttattatgccatcccgtgtctatgactggtgggaccgtctgtgtctatgatttgtgggtccgatggcatattggcgaagcccacaaataataatggcaaaattgccaatggctcagtgaaagagaggccgagcacagctgaccgctgctgcgctgcacaaggaggctgtgtgcctgcacgttttttttttttgcctcagggctcaggctctcgcaatttgggccctaaccaatggacgctaaatcatcggaaaccgctgaccgcatagcaccagtactgctgctgttgctgtctgatgaagcgccctccagttctccactacggcccctaccccacacccctgacaatgcgaccgtttgcttttatcaccacacaagtatactgaaacacacacacacacacacaaaaaggggttcgctgctgacttttcacctggcactgagagccgtaatagcacttcctgctcctgcagcgtgcaataatccgcgttcaagggatgggtaaattggtcggatagatcgcaaatcttatcagatcgatctcgtgccaaggcagccatgccccacccccaccgcgtttggtgcgacacggtccccagccgcgcatatgatatgcgtgtacgagtagtccgcaggttttctctcgccagcaacgccatctcaggacctgcggagcaagtatataaaagcactagcgattgttgtttcacccacaagattccaccatcaccacctcatttgttagcggtaatacctcgcctccgttaccccgtgcttttaattccacgggaaaccgaaaaagaagagagagggaaatgccagcgaaattgcatggtggcctcgaaacgccgtccagactccagagagaaccaggcatggcttcagagagagagagaaagcggcgacaaagaaccaaacgaaaccaaaacaaaaaaaggctttctttgcaaagtcggacgcacaaagcggggagggcgaattccgcacgaaacacgcagaccatatatgccc

SEQ ID NO: 108 - DNA, 1500 bp, Zea mays
  source 1..1500: mol_type = genomic DNA
cccgccctcgtttttcagtaaaaaaaatatggtaatggaagtgggagagagtttttccaactgtttccgatcgttttcatccctatctataaacatccacatgagtaggggaggcggggtggcgagtggacgacactgtagccaacctaaggaccaaagctttagccttaaccattgcaccatgtgtcgcttattgttatatagagtatataaatgtatatagtaacaatttgaaaattaaaattaaaatcatgattgaataaaaatctcatttaaataaaaaattacatatatgatatatagaattcataacaatgtacgagtaactaactagttctatacttaagcataaatagaaagcgtagcaatgtatgcacactttgctagtcggatatttagatactagttagaagtattaaatatagtctaagtataaaactaattatatagatgaggactaaacagcaagacgaacctattaagtttaagtagtccatggttcgtccatgtaaaataaatatttgctaataatagattaattagacttaatagatccatctcgtcgtttagtctttatctatataattacttttgtagttagactatatttaattttagtaattgacatttaaacatccgatatgatccagacttgatgttagtcaggaaaaccaaacatccccttaaccatattggtcccaatttttggtgcctttacccatcaaatgatattcacacaatcacacatctgggcctaactttcatcgttgctgtccacgacggcgacctggaggcgaggtcaattccttggcccaagcatagcttggagcttgcacgctaagaagaggctctcgtactctacaaacagtacagcacatacaggtgacaaaacgacacacatcaaccagccaaataataaatgagcttcttcatgggcacggcaagccgacaactaccaacaagatacaggtgacaaaaagaaaacaagaggcccccactcaccagtgggtcgtaggcaacgcacgcggacgcggtccagcgggcgagaagatccccgacttgcgcccaaagaagatacaggatcaaggatttttaaccgcagttttctattccacgaccttatccacaccagcagattcgaaattcacggacaggcccatggacccggcgaaagccagcggtggttcagcccctgacgtgcgggtcccactctccagccgcaccgcctagagaggcagaggcatcccttcgcgtggaagcaaacgaggcgtgataaagtggggctcctcggtcccggcgttggccgcatcgacactcgccgcgcaccaccaccaccgctgcggctcacggctacgcagcccgctctcccgacccccccgtgccctcctctttttgctactagcacatagagtttcgcccgaatcgatcgccgactgactccgctagggttcggcccgatcgccgcttcgtcctcccggctcccgcggggaccccgccgag

SEQ ID NO: 109 - DNA, 1500 bp, Zea mays
  source 1..1500: mol_type = genomic DNA
ttcgtgtatacatttatttatcctatgtacatgcaatgtaaatgatacatagtgcatattgaccttatttttaacatgtacttctttcaaagcaaaccacccgcgaagtgtcacaatgcatacatccttcggtgtcgagggacctcatcatgaggcccctgcccccctaagtctagcttccaatgtcatttaaggaagaagatgagtttcgaggcctttagggcctctgaggttctaccgaacaatctaggttatctttgtctcttagatcccctgcatcagacccctcttggacctcgaccttggagcccttcaaggttctgaagccctcttgagatctatggctcttaatgtgggaacccaagcttctctcttgactaagtacaagaatgctccacattgaggcatacaactagttgtaacccccgggcaagaagtaccatgggagtatggtcccatagccacaactgtctctacttaagacgaccatgtgatactaatgttccgaataaagcttttgcacatcacattaatgtgatggatgatattgtggtcaagcaggcatatggtagccaccacaaaggatatgaagcatacccaaatccactatgaggagtgtgtcgcgtacctatgccacacctaaatgtgctaatttacctgtaagcattataaaatataagcaattacaccgtatctgatggcatggactcgagtacataactatgatatgatatatttctcactgccgcaagatatgcatattacaatgacatgttcaagcgccattgtggtccgcgtagagtcctcgtctctaacaagatgaagtaatcgagcatgttcactacgaaccaacatgtgatcctcctcttcttcgagctataaaatattcatccagcttaaatttcaaaatatgtgtgtcagagttaaaaaaatattcgattcaaatctaaatatcagttctgtattcatatcctagaatattcgaatctatatttgaattcagattacaaggtagtgaattgtgacatgtattcgttcctatccgatccgtcgtttttgagcactaggtgcggtcactgtgacgcgtggacttggcttcgcccactgccatcgtggacccacgtcatcagcaagtgtccatatccaccacccgacccgacgaccgcttgccgtccgatccgtgtgctcccgagggcaaggatggcatttcgccacgcgagatatttttcggtggcctgcacaggccggcagtgcagcggccaaaacgaggtcaggtcagtcacgctgggccccgcctcacgctcccgtcctgctccgggtcccaacaaagccgtccccgggaggtgctcgtgtgctcgtagcgcggtggcgaccccgatgccccgcatattccactgggcgtccgcgccgtcggatgggatcaggacggccgcggcggccccgcgctcggctataaagacgctgcgggggacgcattccctctccgtgctttcttagagg

SEQ ID NO: 110 - DNA, 1500 bp, Zea mays
  source 1..1500: mol_type = genomic DNA
tgcacccatcgatagcagcctggctcaacagttgaattggtggttgggggctatatataccctccaaccacctccactccaaccatccaagcattcattactgcatattcaatacaagagcaatagacaccactccaaagacacaattcaagtgatcgatccgctcaaagtctacaattcaactctagcgcatttagacttgtgagaggatcatttgtgttttccttgttgctcttgtttgcttggttggctttcttcttcctcattcttgttctcaagaaacttgtaatcaaagcaagagacaccaagtttgtaagtggtccttgcggggtctaagtgatccggttgattaaagagaaagctcactcggtctaggtgaccgtttgagagagggaaagagttgaaagagacccggtctttgtgaccacctcaacggggactaggttctttagaaccgaacttcggtaaaacaaatcattgtgtcatccgcttttattttcttggttgatttgttttcctctctcccccggactcggatttattctaacgctaaccccggcttgtagaattaaatcgtgcgactcaagatatatagaaaaatttacacgactgtcgcatggaaacttttcatggcaccacttgatgtatttcctttcttgatactttccttttcatttttcaattaaagttgttactcattttatctttacggacactgagtatacactaggagcaaacttgttagtaactttatttgttttgtcatctaatcatcaaaaccctcaacttgggggtgatttcacttacaatatgaccaatctcaactcctttacggaatgccgatagacacatattctggacaatcacagtctcccgtgcaaaacgagggtaaacccgtcaatttgcgtatggacgtaccgtccgcacgtgagcacaaaccgtctggtccaacgatcgtcgaccccatttttttgaaccgaattactggaatccgcgtctaagccaccacatctcatgatactatatattaatacagtattatatttagtatatacgatgatatggtaaaataacatatgatactatatattaatacagtattatctttagtatatacgatgatatggtaattttagatattgtgataagaaactatataggttggaaatagcctaaggtgaggcgagtacagccccggcacacaaccaatcacggtgacgctctaggattgggccatttggtgtggcactgtagcgaggcccagctcggtccatgagcagcattctggtcggcttgacagatccatcacgccatcggcaaaaatatctggctctcgagaaccctcccggtcccagcgcggtaggcccacctcgggatccttatcctccggtcggaccgttgtttgcgcggtcgccgcccgatccgatcatgacggcgcccgtcacgtccgtcgcgctataaatctgcggggtagggcttcctcactccctcgtgctctct

SEQ ID NO: 111 - DNA, 1500 bp, Zea mays
  source 1..1500: mol_type = genomic DNA
tttggcctcctctccttcacggatcacccttgcagtcgagcgtactctggccatctgatgatgaatttttttgcagtttttacgaagctgatgttttttacagttttttgacgaagctccctcttttgacgaagctaattaaagatgatggtttgctcaagaatgcagtgaaaaagcttcgactatggttaaatttttcaacagcacaacactgcaatgacaatgaatgttgtggtaacttcacacctacctctctgttttatatagtgctgcaggtgggaaggtgaatggccaagttgcctgcacccgctgaacagttacccgcacccgctgaacggtggaccactcgacgcttgggaggcgaatcgccagaccgtgcgtacccgccgcatggtgggccacctcgtgcttggaatattttaatcgtttcttgacaacgagctcagggaaggtgttttttggatctacggcattccgaagccttggagatttttcacggatcaagctcgttacaaaaaacgatctagcaccgcgaaggagctattgttgggagactacttcatcgccaaaggtcctttaagaagaaacatcttcggaagatcagtacatacaacgcgaaggtacatgccgaagctaccatccagggagcttcggcatagcgacatgccttgagacgaagggctgcaccgacttaaagaggaaaagaccaatcggtccatgataatttgtgtcatggttgtaactaattgccaaggacataaatgtaattctgaccgggctgcgtcctgtgcctataaataggtgaacagtacctctgtactgttcacgctggattgtattcactcgtacgtcacgcttggacctttgccttctgtcaagccgaaggtacaaatacaattcaatgtaattcatgttcatttataatgatataaaaaagatatattaatgatgttatataactattcattttactcctcatgtttcatatgcttcttttttcattaatatataatatgatgatgaaggtacgtgcttcatgaccttcgtctgaagatcattatatcctatgagaaataatgattcgaaggacgaagacccctaaccattaatattttatgttgccttattcttaattcgaagcatttaagaacaagttcccaacattttcgatacctactcattatctattatcaaatttcttctaactaacgattactaaggtgcacggaaaacaaaaatgaaaagtgtttagaggtgaacgccaacaacaaggggtgagaaaaagaaaccgccatgtagtgtgggctgctaggggacggccgtgcccccatgtagccccttatcccctgtgatcggatcatcacaaaatatctttggagcgcggttgatattatcactataattgggggtttacacgaaaaatcgcaccatctagaggtttatgaagcctccaaaaaaatatctaaacaacaactacttcctagtataaagtgacagtag

SEQ ID NO: 112 - DNA, 1500 bp, Zea mays
  source 1..1500: mol_type = genomic DNA
atggagtactcatgtttaataagatttttctttataaagagagccttattaaaactcaacaggtataagagttttaaaaatgatcgtcttcgctcgccttctcattccgctctgtaaatgatcctttggctctcaagttccaactgattgacgatgggacgttgggataaattccaggagacaacattctaatataattaattactcgcaatttaaaggacagagaaaaccatgctacttgcttcgatgtttatttttacaaaaattaaaaacatagagatacagaaaaatctgaccacgagaattgctagactctagcgtcacatgaattgggagttattgtaagtttttgggccaaggtgctagcgtggagagtgagtagagacaagcagcgtgtgtgcttggcaaaataaaagttttactttttcttttgcttcttaaaagactgagaggtgcagtggatgaaatctcggatcttgacaaatgattcgggcctttttgtgttgctttttcttttgcttctttaaagactttgagaggtgcggtgaagcttgagtgcagtaggtttcgaaaggcacgacttacacgaaaaaaatggagaaaaagagaaaacaacaaaaactggagatgcggggtatcgatccccgtacctctcgcatgctaagcgagcgctctaccatctgagctacatccccttttgttattattataaaatagttaatatttggtaataatgagctagactaagttggaccatgttaagaaaaattcattagccaattacttagcatgtgaaaatcatgcgcactggtgtgagatttgtaagaggtatataaagtatctatggtctcatgatattagaaagaggacatgaaaaatcaaaggagtttatagtggaaaaaggaggcggacacaactgcatcgccaaattcatcacacctgcatgcacaaccctgagttgagtgagttccacgtcgtgctctgtagcatagcagaccctgtggtggtactacccgtaacatgttgttggagatgtctaaagtgttggacaacagtgtgccctatgcccctatgtctataggatctcgagcatctcaacgaaggagacagtcaactaatcgctctactagaagtctagttcagccatgatagtaggcccaccatcttacgagtgggacagatgataaagttgttgcttaccaacatccacgcaaggtcatatcccttgatattgaaaagatgtcactgacacatgggacctgcctgtatcaccgagagcggcaagtatgcaatttgcaatggcacgatagtaatcgacattaattaaagcaaaaaaatcaatgtttttttaaacaaaagggctaagagcccaagacagacgacgtagcccaactgaatgccctcctgcagcccagcccaacctgtgcgatcgggtcggtcaagcaacccattcgatcgggtcgccttcagacctgacctctcaaatcaaaccgggaccgg

SEQ ID NO: 113 - DNA, 1500 bp, Zea mays
  source 1..1500: mol_type = genomic DNA
agttaagctaacttccgacaaaaaaaatttcgagagccaaactttcgacgggatggctttacttccgagagtttagctaacttcctagagtttagggctaacttcctagggtttaggctctaggaagttcactattttggtgtagtgggatagatatcccctaggtccactaaaggaataaaagacctcacgaaaggcccaagggcccaataactcgtaaggtcattctttcgtgggcctggggtggaacaaccagcaagggggaacgacatgaggccgattggtgcaaacccgagcggcccacatcgtcgagcgaatgatcgcaacagagacccgattttcccgcgcgggagcccccatgcagcggagccgtgcgaggataagtcggcgaggatcacgcaggataaactcgagaggttcactatcttttagttacttgttgttatcatacccacatgtgttgccccacggtcgaatatataaggcctagggggcaccccttcagaacgatcgaccctatcttacttagccacccacgtaaactctctgtgccttcaatccagagagccctcttgtaaccacgctcgtatactcaccaggacgtagggtgttacgcatctctaagcggcccgaacctgtaaatcttgtccactgtctctcgtgcgatcggcacgaaccattttgctacagtcgttgacaccgtcctactcctaaaaacaccttgaggggcaaccccgggtgtgcggtcggacccaaaacaccgacaccgggcccaagggccggaccgtccgctcattttggtgtccaacaggtcgccttttaatatattgatcaggtagctatgaccgaagaaaatgcacgccctctatatactacctacattcacaaatatatgacacaattgaactttttcgaaaactttgaccactcgttttattcaaaatatttactcaataatgtaaaacttcaagtaagcacaaattatcttaagtgataaaacaaatcacaaaaaatgataacttattattttttgaataagatgagtgatcaaagtttttttaaaaagtcaacgacgtcatattgaacagagggagtattagagttctattaaatatttatattatttcattataaaagtcggtggctaaagttcgtattttgggaacaaatcaagttaattaatttattatttttccgaaagaagcgggtaggacacgaaccaaaagcaggtagctacgggaccccgcgattttagcagatcgcagcaccggcccgcgcacacccaccaaacccggcccgctactgaagtgacgtcactcaagtgtccacggcccggcccacccagtcatagctaactacacgacacccacctgtcttcctcggctcctccaccccggtcctctcctgccgcgtcgcgttcgcccctcgcagcgggcaactcccaccgccgccgcttcgcatccgcccatctgcttccatcgtctcacggaggtcgctcac

SEQ ID NO: 114 - DNA, 1500 bp, Zea mays
  source 1..1500: mol_type = genomic DNA
gaccggcgtcccatctggccagctccgccagatggacaatgatggcgccccacaagctctatgacgacggcggctctcagctctcttacggaagcagggcgacgtcagcaaggactcgaccgctccaacagctgtccctccgccaggctccgtcgctcctccgacagccacgacatcacgccagcaaggtgccaagacctctccggctgccacattggcatgtacctagggcgctagctctctctccgctagacacgtagcactctgctacaccccccattgtacacctggatcctctccttacgactataaaaggaaggaccagggccttcttagaggaggttggccgcgcggggacgaggacgagacaggcgctctcttggggccgctcgcttccctcacccgcgtggacgcttgtaacccccctactgcaagcgcacccgacctgggcgcgggacgaacacgaaggccgcgggatctccacctctctcacgcccgtctccggccacctcgcctctccccccttcgcgctcacccacgcgctcgacccatctgggctggggcacgcagcacactcactcgtcggctcggggaccccccggtctcgaaacgccgacaaaaccgtaggaaataaataacttccgagaggcaactggtagctctaggaaataaacataacttcctacggtattttataaaagctgtaggaagttagttttcacatgctgacccgtgtgttcggtcaagcgagccactaacttcctagaggcggccgtaggaagttagcatgggcagctaacttcctgcggcctcctctagaaagttaacttttaattgctgatccgcgggtgtggtcaaatgagccgctaacttcctacggcctcctctagaaagttagatttcagcttttgaccagccaaacgaaaagctcgtgctcaagattacaggaacaatccaaagattacaccaatcatattaagattggaggatcaagaaaaggagaatctaaatcctaagagctagtttgataacctcgtttttttgacagttctctaccgatgttcgcacacgcagcagcccttgttgccattttttctccgcgtcctctgtgccccaggtagggatgctgccctttataaactttcacctccaactcactcgtatctcctttcgagagattctgatattttccatcaacaagaaacagatttgtaaaattatcatcaggccacatttcatagacccagctggacccacaatttataaacacagtggtaattataacaagaaaacaacattgtgagtggcaaaaatctaattgtctcatctcctttccgttcgccaattcgaaatcgaatccgttccctaattcgaaatcgaatggggtcggtcttgtcgtgcgtgcgtggctgcgcgagcgacttgactgcacccgacccccctcctcccagtccccacacactgcacgccgccgccgggtcctcctagggtttcgccgcg

SEQ ID NO: 115 - DNA, 1500 bp, Zea mays
  source 1..1500: mol_type = genomic DNA
aactggactagattcgtctcgtcttttaatcttcggctgacaaattagttttataatccgactacatttaatacccgaaacggaggttcaaacattcgatgggacaggggctaaattttagaggggtgtaaccaaacacccccgtagtccaaaactgcaggttaatgggtctatgacctaattttttgggacaccaaaacataaaaatttggaaaacaaatatattctctagactcataggaacccctatagattttcccaaattatttttgatttttaaaattcaatcttttgaaccgaaaaaattcaaaattttacacagatcttgattctgtgcagtgctggtgatgggaaaaagcgaaaaaccatcggtatgtttttgacaaatatgaaaatgggacaaaaacaacatgtgtgttttttcgaccgtttccgcttttcttgttttagtcacaatagctcgtttttatccacatatgatatctcattttagataatacatgaacaaatcataattgattatatcatatctcaacaaattaacccgtaatgaattatttttctttgatagtcatatgtacattacaatatttcgcttccatatgtatggatgtgatgttttaatcgattgcaacactacttttatttttatactctatgtgacaattatttccgcttttatttacatcttattccgatctgttatcgatatcgatttgttccgtcccgtttttatcttatttctgatagttccaatttaatcttattttcgaaataaagtatgaaaataaaaataagagagattgttacgttcgatccggttttgaaccctagctatacttgcccgttgttgcaactggccggccattccataggcgggcacagtcagcactcagcagtgacagagtgcgcgtgcgacacacagtttcaaatttcaaaactgaaacgggcggctataaacagaacccgctgctcccaggagcctcacgcagataaattcacccacatcaatggggcccaaatatttataaccatctattggtcccacatgttcgtgtcacaacatcctctaccgcaggtaaagatagccgtctcgccaagaccccgagcccgccggctccgcgggacccgccgccagctcacacccaccgttgccggccgctgagccgttcgaagccaaaacggtcgttaaccacccaggctgcgccgtcggctaccatcacgccgttagccccgaaccagacggcggctaggtcttccgcgccgcgccgcgccatcacgggccggccgcggccgcctctcccacgctgcctataaaagccgccgcgaggctgagcagcattatcgcttcagctcggcgtcttcacaaacgccggcgcaaactctcgcccgagcccgacagatcttcaattccccattccgcccaccgatcgaccttcacgccagtctcggtctcttccgaaggcgtcgcgcgcggttgtttgagaggagaggaggaag

SEQ ID NO: 116 - DNA, 1500 bp, Zea mays
  source 1..1500: mol_type = genomic DNA
atgagctattatccataaaattatctaatattcattattattccataaattgatcatttttgcaaggctcgcgagctggaacgagccggctcggctcggctcgctgcaaaaacgagctcgaaacaggagctcagctcggctcgtttgaggctcgcgagccgctccgagctcgagccggctcgcgagcctcgagctaattttccaaccctagcccctggccagtgagctgggcttccccgtgagcgtccaacggctcccctcccccctcacgctcttctcggtgagagctctcaccccctctccgctaatcagctataataattacaaaattaatttttagatttacttagcagataacaatatgtattataacactacaaaaaattgtataatcatttaaaattccaaaaaccgatgtataaaagtcaaataacccagggttaaggggacttgaataggaaaatgattggatatgaggaaaaataagggacagatatttgaggagatagatatttaaatataaatagaaattatgaatgtagggatttaggagggggaatggttcaaaatagcctaagaataagagtttgccacctctcttgagccatcgtccgctcgctggcaggagtggatatatggccatatgggtgtttgttggggtcagttgaccccgataaaatttataaatctttaaataaaacactaaagtttgaaaaaacattgcatcatataatgaagctgaccctattccgacatcattctttgctaaattcgccattgctcgctcatgcctaaaaaaagacagagtaagcacgttggggggtgcttggttcttttagcagcacaggctagcatggtaaggctgcctaatcttgctcagcttggccagcaaatataaccatggacaatttaaatagcacaacgacatgcatgtcgtgactgaaatagtacaggaaggcccacccgtcggccagcctccaatcgcagaacgtggtagctttctccgtccgctcgccttgccagcgcgggagagccgaattggccgcccgtccgcttcaacgacgaggaaaagctagcttgcccagacaagttagcttgctaagcaacgcaaggttctaaacacaactagtaccaaacaccccgtcgctaatgcagtatccaagaagatcattagcttatccaatgctgaaggcaaatgacacgtcagtgcaagctcactcacttgatccagatctttccatgcctatgcctataaccctaaataacggagataagctaaaagatatttatctcctgggcccacctgccatcccgggcccaccgcaccagcaaggcctgcaactttcacacctaacccttccaagttaactgcattcacgacccacccccccttcttctctctcctggccacctccgtccattccacactccctcctccggacgcaccttccccgctatataaggcacgcgccccgagcacaggcgaagagcgtgcagagc

SEQ ID NO: 117 - DNA, 1500 bp, Zea mays
  source 1..1500: mol_type = genomic DNA
gtctttgagctgaggtcttcctgagagaaggtgattgtaatgtgtgaccatttggatttgatgaagggtccttatactccaacatgttgtaccattctctgtgcttccttcttctgcttcttgttagctggctcagaacttgaacctccgtgattgggagcaccagcttcgtagccgaaggtgtcacaacttgatcaccttgcgaagccattgtcgtggtcgtggaagtgagttctccggaggtgggcaccaatgttggtcacttgttctcgaatgctgtgaattaagaacaaggcaacacagtcgctagggattaaagaccttcgtcctccgaaacattgtttcctcttggattcaatgatcatcggacgaaggccatgaaggacatgccttcatcatatcataaataaataaaaatgtaaagagataaatacattgatgattactctttaatacattcatacttgtactccgtaaaacatgtataaatatcaataaaattcacgttatattgatacattcggcttgctcgaaggtgaagatgcgagcgagtgattacaattcagcgtgaacagtagggtgttattgttcatctatttataggcacgggacgcatcccaggggaaattacattcacgaccctcaacattcatctagagacaacctagattaacaaggtctatctggtcttttcttcttctgcttgacttgaacagaaactaaatggtagctttgacatttgactatgttgattctacattaagtctgtcttgagaattttcggcagaaaaaaagtagacttatgtaccattttaccgaagatgtttttgttgaaaacttttgtcggaaaagaagacacccaacactttacgtcgccgccataagtcaccagcgtcgtaccatgacttatggcgtctaaaaaagattcaaagaatcatattacatcctttataaaaccaaacgtatattcttttagtaaaaaattaaaatgcaaaaaaaaatggtttcccacagccctagcaaaacacaatcgccacttcattccaccccccgtttcgatgatgggtggggacaaaaatacagggacccacatgtcagttaaaatctgaccatgcatgaggtcatggcacggaatagttctttactactaggcaaccagcacgttgtacactgactgtggggccaaccgtaccttgggtccacaagtcactgtgtgcttgacaacacagcattggcagatccatgtgtacgttctcagcgccaggaaattgcggccgtcctttcctccttttccgttaagccgataatcttcaatgacgcacgggtccaagattagcttgggcccatatggcagtgtgggaagctgaatccggacgcgtgacgcgaggtctcgcgtggtgcggcgggcactacgtcgtggatggggaaggggattaaatatcgtcggacgaggcgtggccaacccctttcgctcctctcgccgctttgggttggggttta

SEQ ID NO: 118 - DNA, 1329 bp, Zea mays
  source 1..1329: mol_type = genomic DNA
gtaactgcgatcatccatcctcccgcttccactctcccttcacctcctctgcttgctaggtatacgaacatacgatttattacgggttatatgggggcttcgattcccagatctggcgatctattatcgtagctccgagtcctcgatctagtaattgtgggatatgcttgtaagaggctctgagatgggttgggttgggttgggtcgctgtgacgattccaacagcctcgtttcttagggttggatcttctcgtggtttcctttttaattaaataagtacctgatgcagaatggtgcgtcctattagatggaaccttgatcttgatgcatctaaccttgatcttgttcgctgtgatgattccaacaggctcgtttcttaggcctgttcgtctggttcgtcagatcagtttcgttgcttttggcctcgttgtaaggtccatccagatcggagtagaatcgaatgatttattatacggtagctgctggtctcattagatttggatctgcatgggttgaacatatgtattcataattaatatggtgtatacgtactagtttgctggtcttatttttttagcctgattgcttctgcctttctggcaacgcctgatccacgcgttagctagagtggattttagttccttgtttacgcggccacacctgccgcctagaaaagctgcagcgagaactctaattaaatttggatctacatgtgctagcatatatgtttgtaattaatatgatggatgaatatgtgcttcagagttgagttcctgttgatgctgtagttctgcctgaattgttgaggctgtagcttctgcctgattaaaatgcaccgtgcctatctgttaaactctagggtgtgtgatttagccggtgacggtggtttaatatgtgtaatttcactgcttatagtaatgcaattcacctttgcttgaacatgcattgtcttgttgctttgttctatacacatgcttagctattatctgatgagcatgcactgttttgttctgtttgatatgcatgctcagaaatatgtagatgtgtggctcctgctcggttgttctttatcatccacctgttgaacatgcatgttcttgtcgcttatctttattatatattaccttcgttctcgaatatttgtcgcccgctagttcatttttgaactaaaccgtgacaaataaaatagaacgtagggagtggcatcatgctgctactgtaccttacggtggcaactacatcttgagcacgcatatatcttatagtgttccttttcttttcctccttggtctactgttatatgcttacctttttttggtttccttgcag

SEQ ID NO: 119 - DNA, 1076 bp, Zea mays
  source 1..1076: mol_type = genomic DNA
gacggagatcaacgtccctatttacattataattaggaaatgcatcctttgttattaataaaaacactttcacttatatatattgttagatgtaagaaatcattatgggtatattaaaataaacatatttgtacaatgattgatctcttacccaaataattatttgtttttattattagctagtatacgaaaacatcaccacgtacaggtttgacggattcccacagaaacagggatgaaaaatacttctacatccctgtcccgtttacccatctgagaaagcgggaaatcgggcataggatccattgccaaagatcgtagggctataacctaagcgttgcaacgaagcgaagcagacggtggagacgttgacgcaaagcaatgaacttgaacggcatctctctcgctggccctggccttctcgaaggctctgcgtgggtccttgcgcagttgcgccgcagcgggctggcagcatccggaaattgcgtcttgcgtggcggagcagacactaaggtactattttacgttctatttagttggactgtggcggtaaactatgaaaaaaactattgcagactatgagctattaaaaagctaaaaattatttagtgtaaaccactaaaaaccattaaaaattctttgatatatattttcacagttttataaaaaatccactaaaaacaggtcaaataagctttcaattttacactacgaaaaagtcagcttttaaaaaaaactgcttaaatccagtcctttagtttaatttttatcttttaggaaacaaaagccaaaactaaaaccaaaccaaacctacctttaaaaccgatctaataggaacgcggtgtttggaacaactagatattaattttagaggttagaccgccacgaaagcgtcactgcacacggcattcccctcccctagcgttatcgtcgcaccataaataaccatcctctcctcgcctttccccacatctcatcttcgtctgtgttcttgggcgtacgcggacacagccccgatccgaatcgtcgtccttgcgagcctcgccgatcccccactcccctcccctcgcttcaag

SEQ ID NO: 120 - DNA, 42 bp, synthetic construct
  misc_feature 1..42: note = TS2 fwd primer
  source 1..42: mol_type = other DNA
cctccgcttcaagcgatcgcaggtaactgcgatcatccatcc

SEQ ID NO: 121 - DNA, 46 bp, synthetic construct
  misc_feature 1..46: note = TS2 rev primer
  source 1..46: mol_type = other DNA
aggctaagttaaagtcgggtacctgcaaggaaaccaaaaaaaggta

SEQ ID NO: 122 - DNA, 42 bp, synthetic construct
  misc_feature 1..42: note = pTS2 fwd primer
  source 1..42: mol_type = other DNA
tcgaagcttggcgcgccgacggagatcaacgtccctatttac

SEQ ID NO: 123 - DNA, 42 bp, synthetic construct
  misc_feature 1..42: note = pTS2_TS2 rev primer
  source 1..42: mol_type = other DNA
acaggacggaccatggctgcaaggaaaccaaaaaaaggtaag

SEQ ID NO: 124 - DNA, 42 bp, synthetic construct
  misc_feature 1..42: note = pTS2 reverse primer
  source 1..42: mol_type = other DNA
acaggacggaccatggccttgaagcgaggggaggggagtggg

SEQ ID NO: 125 - DNA, 33 bp, synthetic construct
  misc_feature 1..33: note = pTS1 fwd primer
  source 1..33: mol_type = other DNA
cttggcgcgcctgccacgcaaactaaaaggcaa

SEQ ID NO: 126 - DNA, 42 bp, synthetic construct
  misc_feature 1..42: note = pTS1 rev primer
  source 1..42: mol_type = other DNA
cctgcgatcgcggtggcaagctcgagatggggagccgctact

SEQ ID NO: 127 - DNA, 39 bp, synthetic construct
  misc_feature 1..39: note = pTS27 fwd primer
  source 1..39: mol_type = other DNA
tcgaagcttggcgcgccccggctataccgctcccgccct

SEQ ID NO: 128 - DNA, 42 bp, synthetic construct
  misc_feature 1..42: note = pTS27 rev primer
  source 1..42: mol_type = other DNA
ggatgcctcacctgcgatcgcaggacgaagcggcgatcgggc

SEQ ID NO: 129 - DNA, 21 bp, synthetic construct
  misc_feature 1..21: note = GUS-F primer
  source 1..21: mol_type = other DNA
cttacgtggcaaaggattcga

SEQ ID NO: 130 - DNA, 20 bp, synthetic construct
  misc_feature 1..20: note = GUS-R primer
  source 1..20: mol_type = other DNA
gccccaatccagtccattaa

SEQ ID NO: 131 - DNA, 21 bp, synthetic construct
  misc_feature 1..21: note = Gr5-F primer
  source 1..21: mol_type = other DNA
ggcagtttggttgatgctcat

SEQ ID NO: 132 - DNA, 26 bp, synthetic construct
  misc_feature 1..26: note = Gr5-R primer
  source 1..26: mol_type = other DNA
tgctgtatatctttgctttgaaccat

SEQ ID NO: 133 - DNA, 23 bp, synthetic construct
  misc_feature 1..23: note = GUS probe
  source 1..23: mol_type = other DNA
aacgtgctgatggtgcacgacca

SEQ ID NO: 134 - DNA, 17 bp, synthetic construct
  misc_feature 1..17: note = Gr5 probe
  source 1..17: mol_type = other DNA
ttgaagtcacaaagcca

SEQ ID NO: 135 - DNA, 50976 bp, synthetic construct
  misc_feature 1..50976: note = vector sequence
  source 1..50976: mol_type = other DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttacccggaccggaattcgagctcggtacgatatcaacaagtttgtacaaaaaagcaggtttaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacacccccgccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagacagctttcttgtacaaagtggtcgatatcgatccaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactgccaggtggtggccaatggtgatgtcagcgttgaactgcgtgatgcggatcaacaggtggttgcaactggacaaggcactagcgggactttgcaagtggtgaatccgcacctctgccaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggtgacgtggggcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgcgatctgagcttctagagtcgacctgcaggcatgcccgcggatatcgatgggccccggccgaagcttcggtccgggtcacctttgtccaccaagatggaactgcggccgctcattaattaagtcaggcgcgcctctagttgaagacacgttcatgtcttcatcgtaagaagacactcagtagtcttcggccagaatggccatctggattcagcaggcctagaaggccatttaaatcctgaggatctggtcttcctaaggacccgggatatcggaccgattaaactttaattcggtccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgaccgccggggatccacacgacaccatggctattgaggttaagcctatcaacgcagaggatacctatgaccttaggcatagagtgctcagaccaaaccagcctatcgaagcctgcatgtttgagtctgaccttactaggagtgcatttcaccttggtggattctacggaggtaaactgatttccgtggcttcattccaccaagctgagcactctgaacttcaaggtaagaagcagtaccagcttagaggtgtggctaccttggaaggttatagagagcagaaggctggttccagtctcgtgaaacacgctgaagagattctcagaaagagaggtgctgacatgatctggtgtaatgccaggacatctgcttcaggatactacaggaagttgggattcagtgagcaaggagaggtgttcgatactcctccagttggacctcacatcctgatgtataagaggatcacataagagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacagtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcggagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccactcttcattaaccgctatatcgaaaattgcttgcggcttgttagaattgccatgacgtacctcggtgtcacgggtaagattaccgataaactggaactgattatggctcatatcgaaagtctccttgagaaaggagactctagtttagctaaacattggttccgctgtcaagaactttagcggctaaaattttgcgggccgcgaccaaaggtgcgaggggcggcttccgctgtgtacaaccagatatttttcaccaacatccttcgtctgctcgatgagcggggcatgacgaaacatgagctgtcggagagggcaggggtttcaatttcgtttttatcagacttaaccaacggtaaggccaacccctcgttgaaggtgatggaggccattgccgacgccctggaaactcccctacctcttctcctggagtccaccgaccttgaccgcgaggcactcgcggagattgcgggtcatcctttcaagagcagcgtgccgcccggatacgaacgcatcagtgtggttttgccgtcacataaggcgtttatcgtaaagaaatggggcgacgacacccgaaaaaagctgcgtggaaggctctgacgccaagggttagggcttgcacttccttctttagccgctaaaacggccccttctctgcgggccgtcggctcgcgcatcatatcgacatcctcaacggaagccgtgccgcgaatggcatcgggcgggtgcgctttgacagttgttttctatcagaacccctacgtcgtgcggttcgattagctgtttgtcttgcaggctaaacactttcggtatatcgtttgcctgtgcgataatgttgctaatgatttgttgcgtaggggttactgaaaagtgagcgggaaagaagagtttcagaccatcaaggagcgggccaagcgcaagctggaacgcgacatgggtgcggacctgttggccgcgctcaacgacccgaaaaccgttgaagtcatgctcaacgcggacggcaaggtgtggcacgaacgccttggcgagccgatgcggtacatctgcgacatgcggcccagccagtcgcaggcgattatagaaacggtggccggattccacggcaaagaggtcacgcggcattcgcccatcctggaaggcgagttccccttggatggcagccgctttgccggccaattgccgccggtcgtggccgcgccaacctttgcgatccgcaagcgcgcggtcgccatcttcacgctggaacagtacgtcgaggcgggcatcatgacccgcgagcaatacgaggtcattaaaagcgccgtcgcggcgcatcgaaacatcctcgtcattggcggtactggctcgggcaagaccacgctcgtcaacgcgatcatcaatgaaatggtcgccttcaacccgtctgagcgcgtcgtcatcatcgaggacaccggcgaaatccagtgcgccgcagagaacgccgtccaataccacaccagcatcgacgtctcgatgacgctgctgctcaagacaacgctgcgtatgcgccccgaccgcatcctggtcggtgaggtacgtggccccgaagcccttgatctgttgatggcctggaacaccgggcatgaaggaggtgccgccaccctgcacgcaaacaaccccaaagcgggcctgagccggctcgccatgcttatcagcatgcacccggattcaccgaaacccattgagccgctgattggcgaggcggttcatgtggtcgtccatatcgccaggacccctagcggccgtcgagtgcaagaaattctcgaagttcttggttacgagaacggccagtacatcaccaaaaccctgtaaggagtatttccaatgacaacggctgttccgttccgtctgaccatgaatcgcggcattttgttctaccttgccgtgttcttcgttctcgctctcgcgttatccgcgcatccggcgatggcctcggaaggcaccggcggcagcttgccatatgagagctggctgacgaacctgcgcaactccgtaaccggcccggtggccttcgcgctgtccatcatcggcatcgtcgtcgccggcggcgtgctgatcttcggcggcgaactcaacgccttcttccgaaccctgatcttcctggttctggtgatggcgctgctggtcggcgcgcagaacgtgatgagcaccttcttcggtcgtggtgccgaaatcgcggccctcggcaacggggcgctgcaccaggtgcaagtcgcggcggcggatgccgtgcgtgcggtagcggctggacggctcgcctaatcatggctctgcgcacgatccccatccgtcgcgcaggcaaccgagaaaacctgttcatgggtggtgatcgtgaactggtgatgttctcgggcctgatggcgtttgcgctgattttcagcgcccaagagctgcgggccaccgtggtcggtctgatcctgtggttcggggcgctctatgcgttccgaatcatggcgaaggccgatccgaagatgcggttcgtgtacctgcgtcaccgccggtacaagccgtattacccggcccgctcgaccccgttccgcgagaacaccaatagccaagggaagcaataccgatgatccaagcaattgcgattgcaatcgcgggcctcggcgcgcttctgttgttcatcctctttgcccgcatccgcgcggtcgatgccgaactgaaactgaaaaagcatcgttccaaggacgccggcctggccgatctgctcaactacgccgctgtcgtcgatgacggcgtaatcgtgggcaagaacggcagctttatggctgcctggctgtacaagggcgatgacaacgcaagcagcaccgaccagcagcgcgaagtagtgtccgcccgcatcaaccaggccctcgcgggcctgggaagtgggtggatgatccatgtggacgccgtgcggcgtcctgctccgaactacgcggagcggggcctgtcggcgttccctgaccgtctgacggcagcgattgaagaagagcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatctactgtaatggcgaaaattcacatggttttgcagggcaagggcggggtcggcaagtcggccatcgccgcgatcattgcgcagtacaagatggacaaggggcagacacccttgtgcatcgacaccgacccggtgaacgcgacgttcgagggctacaaggccctgaacgtccgccggctgaacatcatggccggcgacgaaattaactcgcgcaacttcgacaccctggtcgagctgattgcgccgaccaaggatgacgtggtgatcgacaacggtgccagctcgttcgtgcctctgtcgcattacctcatcagcaaccaggtgccggctctgctgcaagaaatggggcatgagctggtcatccataccgtcgtcaccggcggccaggctctcctggacacggtgagcggcttcgcccagctcgccagccagttcccggccgaagcgcttttcgtggtctggctgaacccgtattgggggcctatcgagcatgagggcaagagctttgagcagatgaaggcgtacacggccaacaaggcccgcgtgtcgtccatcatccagattccggccctcaaggaagaaacctacggccgcgatttcagcgacatgctgcaagagcggctgacgttcgaccaggcgctggccgatgaatcgctcacgatcatgacgcggcaacgcctcaagatcgtgcggcgcggcctgtttgaacagctcgacgcggcggccgtgctatgagcgaccagattgaagagctgatccgggagattgcggccaagcacggcatcgccgtcggccgcgacgacccggtgctgatcctgcataccatcaacgcccggctcatggccgacagtgcggccaagcaagaggaaatccttgccgcgttcaaggaagagctggaagggatcgcccatcgttggggcgaggacgccaaggccaaagcggagcggatgctgaacgcggccctggcggccagcaaggacgcaatggcgaaggtaatgaaggacagcgccgcgcaggcggccgaagcgatccgcagggaaatcgacgacggccttggccgccagctcgcggccaaggtcgcggacgcgcggcgcgtggcgatgatgaacatgatcgccggcggcatggtgttgttcgcggccgccctggtggtgtgggcctcgttatgaatcgcagaggcgcagatgaaaaagcccggcgttgccgggctttgtttttgcgttagctgggcttgtttgacaggcccaagctctgactgcgcccgcgctcgcgctcctgggcctgtttcttctcctgctcctgcttgcgcatcagggcctggtgccgtcgggctgcttcacgcatcgaatcccagtcgccggccagctcgggatgctccgcgcgcatcttgcgcgtcgccagttcctcgatcttgggcgcgtgaatgcccatgccttccttgatttcgcgcaccatgtccagccgcgtgtgcagggtctgcaagcgggcttgctgttgggcctgctgctgctgccaggcggcctttgtacgcggcagggacagcaagccgggggcattggactgtagctgctgcaaacgcgcctgctgacggtctacgagctgttctaggcggtcctcgatgcgctccacctggtcatgctttgcctgcacgtagagcgcaagggtctgctggtaggtctgctcgatgggcgcggattctaagagggcctgctgttccgtctcggcctcctgggccgcctgtagcaaatcctcgccgctgttgccgctggactgctttactgccggggactgctgttgccctgctcgcgccgtcgtcgcagttcggcttgcccccactcgattgactgcttcatttcgagccgcagcgatgcgatctcggattgcgtcaacggacggggcagcgcggaggtgtccggcttctccttgggtgagtcggtcgatgccatagccaaaggtttccttccaaaatgcgtccattgctggaccgtgtttctcattgatgcccgcaagcatcttcggcttgaccgccaggtcaagcgcgccttcatgggcggtcatgacggacgccgccatgaccttgccgccgttgttctcgatgtagccgcgtaatgaggcaatggtgccgcccatcgtcagcgtgtcatcgacaacgatgtacttctggccggggatcacctccccctcgaaagtcgggttgaacgccaggcgatgatctgaaccggctccggttcgggcgaccttctcccgctgcacaatgtccgtttcgacctcaaggccaaggcggtcggccagaacgaccgccatcatggccggaatcttgttgttccccgccgcctcgacggcgaggactggaacgatgcggggcttgtcgtcgccgatcagcgtcttgagctgggcaacagtgtcgtccgaaatcaggcgctcgaccaaattaagcgccgcttccgcgtcgccctgcttcgcagcctggtattcaggctcgttggtcaaagaaccaaggtcgccgttgcgaaccaccttcgggaagtctccccacggtgcgcgctcggctctgctgtagctgctcaagacgcctccctttttagccgctaaaactctaacgagtgcgcccgcgactcaacttgacgctttcggcacttacctgtgccttgccacttgcgtcataggtgatgcttttcgcactcccgatttcaggtactttatcgaaatctgaccgggcgtgcattacaaagttcttccccacctgttggtaaatgctgccgctatctgcgtggacgatgctgccgtcgtggcgctgcgacttatcggccttttgggccatatagatgttgtaaatgccaggtttcagggccccggctttatctaccttctggttcgtccatgcgccttggttctcggtctggacaattctttgcccattcatgaccaggaggcggtgtttcattgggtgactcctgacggttgcctctggtgttaaacgtgtcctggtcgcttgccggctaaaaaaaagccgacctcggcagttcgaggccggctttccctagagccgggcgcgtcaaggttgttccatctattttagtgaactgcgttcgatttatcagttactttcctcccgctttgtgtttcctcccactcgtttccgcgtctagccgacccctcaacatagcggcctcttcttgggctgcctttgcctcttgccgcgcttcgtcacgctcggcttgcaccgtcgtaaagcgctcggcctgcctggccgcctcttgcgccgccaacttcctttgctcctggtgggcctcggcgtcggcctgcgccttcgctttcaccgctgccaactccgtgcgcaaactctccgcttcgcgcctggtggcgtcgcgctcgccgcgaagcgcctgcatttcctggttggccgcgtccagggtcttgcggctctcttctttgaatgcgcgggcgtcctggtgagcgtagtccagctcggcgcgcagctcctgcgctcgacgctccacctcgtcggcccgctgcgtcgccagcgcggcccgctgctcggctcctgccagggcggtgcgtgcttcggccagggcttgccgctggcgtgcggccagctcggccgcctcggcggcctgctgctctagcaatgtaacgcgcgcctgggcttcttccagctcgcgggcctgcgcctcgaaggcgtcggccagctccccgcgcacggcttccaactcgttgcgctcacgatcccagccggcttgcgctgcctgcaacgattcattggcaagggcctgggcggcttgccagagggcggccacggcctggttgccggcctgctgcaccgcgtccggcacctggactgccagcggggcggcctgcgccgtgcgctggcgtcgccattcgcgcatgccggcgctggcgtcgttcatgttgacgcgggcggccttacgcactgcatccacggtcgggaagttctcccggtcgccttgctcgaacagctcgtccgcagccgcaaaaatgcggtcgcgcgtctctttgttcagttccatgttggctccggtaattggtaagaataataatactcttacctaccttatcagcgcaagagtttagctgaacagttctcgacttaacggcaggttttttagcggctgaagggcaggcaaaaaaagccccgcacggtcggcgggggcaaagggtcagcgggaaggggattagcgggcgtcgggcttcttcatgcgtcggggccgcgcttcttgggatggagcacgacgaagcgcgcacgcgcatcgtcctcggccctatcggcccgcgtcgcggtcaggaacttgtcgcgcgctaggtcctccctggtgggcaccaggggcatgaactcggcctgctcgatgtaggtccactccatgaccgcatcgcagtcgaggccgcgttccttcaccgtctcttgcaggtcgcggtacgcccgctcgttgagcggctggtaacgggccaattggtcgtaaatggctgtcggccatgagcggcctttcctgttgagccagcagccgacgacgaagccggcaatgcaggcccctggcacaaccaggccgacgccgggggcaggggatggcagcagctcgccaaccaggaaccccgccgcgatgatgccgatgccggtcaaccagcccttgaaactatccggccccgaaacacccctgcgcattgcctggatgctgcgccggatagcttgcaacatcaggagccgtttcttttgttcgtcagtcatggtccgccctcaccagttgttcgtatcggtgtcggacgaactgaaatcgcaagagctgccggtatcggtccagccgctgtccgtgtcgctgctgccgaagcacggcgaggggtccgcgaacgccgcagacggcgtatccggccgcagcgcatcgcccagcatggccccggtcagcgagccgccggccaggtagcccagcatggtgctgttggtcgccccggccaccagggccgacgtgacgaaatcgccgtcattccctctggattgttcgctgctcggcggggcagtgcgccgcgccggcggcgtcgtggatggctcgggttggctggcctgcgacggccggcgaaaggtgcgcagcagctcgttatcgaccggctgcggcgtcggggccgccgccttgcgctgcggtcggtgttccttcttcggctcgcgcagcttgaacagcatgatcgcggaaaccagcagcaacgccgcgcctacgcctcccgcgatgtagaacagcatcggattcattcttcggtcctccttgtagcggaaccgttgtctgtgcggcgcgggtggcccgcgccgctgtctttggggatcagccctcgatgagcgcgaccagtttcacgtcggcaaggttcgcctcgaactcctggccgtcgtcctcgtacttcaaccaggcatagccttccgccggcggccgacggttgaggataaggcgggcagggcgctcgtcgtgctcgacctggacgatggcctttttcagcttgtccgggtccggctccttcgcgcccttttccttggcgtccttaccgtcctggtcgccgtcctcgccgtcctggccgtcgccggcctccgcgtcacgctcggcatcagtctggccgttgaaggcatcgacggtgttgggatcgcggcccttctcgtccaggaactcgcgcagcagcttgaccgtgccgcgcgtgatttcctgggtgtcgtcgtcaagccacgcctcgacttcctccgggcgcttcttgaaggccgtcaccagctcgttcaccacggtcacgtcgcgcacgcggccggtgttgaacgcatcggcgatcttctccggcaggtccagcagcgtgacgtgctgggtgatgaacgccggcgacttgccgatttccttggcgatatcgcctttcttcttgcccttcgccagctcgcggccaatgaagtcggcaatttcgcgcggggtcagctcgttgcgttgcaggttctcgataacctggtcggcttcgttgtagtcgttgtcgatgaacgccgggatggacttcttgccggcccacttcgagccacggtagcggcgggcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgctgaattgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtcaatgaaagtttccaacgcgagccattcgcgagagccttgagtccacgctagatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgactctagagctcgttcctcgaggaacggtacctgcggggaagcttacaataatgtgtgttgttaagtcttgttgcctgtcatcgtctgactgactttcgtcataaatcccggcctccgtaacccagctttgggcaagctcacggatttgatccggcggaacgggaatatcgagatgccgggctgaacgctgcagttccagctttccctttcgggacaggtactccagctgattgattatctgctgaagggtcttggttccacctcctggcacaatgcgaatgattacttgagcgcgatcgggcatccaattttctcccgtcaggtgcgtggtcaagtgctacaaggcacctttcagtaacgagcgaccgtcgatccgtcgccgggatacggacaaaatggagcgcagtagtccatcgagggcggcgaaagcctcgccaaaagcaatacgttcatctcgcacagcctccagatccgatcgagggtcttcggcgtaggcagatagaagcatggatacattgcttgagagtattccgatggactgaagtatggcttccatcttttctcgtgtgtctgcatctatttcgagaaagcccccgatgcggcgcaccgcaacgcgaattgccatactatccgaaagtcccagcaggcgcgcttgataggaaaaggtttcatactcggccgatcgcagacgggcactcacgaccttgaacccttcaactttcagggatcgatgctggttgatggtagtctcactcgacgtggctctggtgtgttttgacatagcttcctccaaagaaagcggaaggtctggatactccagcacgaaatgtgcccgggtagacggatggaagtctagccctgctcaatatgaaatcaacagtacatttacagtcaatactgaatatacttgctacatttgcaattgtcttataacgaatgtgaaataaaaatagtgtaacaacgcttttactcatcgataatcacaaaaacatttatacgaacaaaaatacaaatgcactccggtttcacaggataggcgggatcagaatatgcaacttttgacgttttgttctttcaaagggggtgctggcaaaaccaccgcactcatgggcctttgcgctgctttggcaaatgacggtaaacgagtggccctctttgatgccgacgaaaaccggcctctgacgcgatggagagaaaacgccttacaaagcagtactgggatcctcgctgtgaagtctattccgccgacgaaatgccccttcttgaagcagcctatgaaaatgccgagctcgaaggatttgattatgcgttggccgatacgcgtggcggctcgagcgagctcaacaacacaatcatcgctagctcaaacctgcttctgatccccaccatgctaacgccgctcgacatcgatgaggcactatctacctaccgctacgtcatcgagctgctgttgagtgaaaatttggcaattcctacagctgttttgcgccaacgcgtcccggtcggccgattgacaacatcgcaacgcaggatgtcagagacgctagagagccttccagttgtaccgtctcccatgcatgaaagagatgcatttgccgcgatgaaagaacgcggcatgttgcatcttacattactaaacacgggaactgatccgacgatgcgcctcatagagaggaatcttcggattgcgatggaggaagtcgtggtcatttcgaaactgatcagcaaaatcttggaggcttgaagatggcaattcgcaagcccgcattgtcggtcggcgaagcacggcggcttgctggtgctcgacccgagatccaccatcccaacccgacacttgttccccagaagctggacctccagcacttgcctgaaaaagccgacgagaaagaccagcaacgtgagcctctcgtcgccgatcacatttacagtcccgatcgacaacttaagctaactgtggatgcccttagtccacctccgtccccgaaaaagctccaggtttttctttcagcgcgaccgcccgcgcctcaagtgtcgaaaacatatgacaacctcgttcggcaatacagtccctcgaagtcgctacaaatgattttaaggcgcgcgttggacgatttcgaaagcatgctggcagatggatcatttcgcgtggccccgaaaagttatccgatcccttcaactacagaaaaatccgttctcgttcagacctcacgcatgttcccggttgcgttgctcgaggtcgctcgaagtcattttgatccgttggggttggagaccgctcgagctttcggccacaagctggctaccgccgcgctcgcgtcattctttgctggagagaagccatcgagcaattggtgaagagggacctatcggaacccctcaccaaatattgagtgtaggtttgaggccgctggccgcgtcctcagtcaccttttgagccagataattaagagccaaatgcaattggctcaggctgccatcgtccccccgtgcgaaacctgcacgtccgcgtcaaagaaataaccggcacctcttgctgtttttatcagttgagggcttgacggatccgcctcaagtttgcggcgcagccgcaaaatgagaacatctatactcctgtcgtaaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgcaagaaactctctcgtactaaacggcttagcgataaaatcacttgctcctagctcgagtgcaacaactttatccgtctcctcaaggcggtcgccactgataattatgattggaatatcagactttgccgccagatttcgaacgatctcaagcccatcttcacgacctaaatttagatcaacaaccacgacatcgaccgtcgcggaagagagtactctagtgaactgggtgctgtcggctaccgcggtcactttgaaggcgtggatcgtaaggtattcgataataagatgccgcatagcgacatcgtcatcgataagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacgaaaatgcgaaccaagtatttcaattttatgacaaaagttctcaatcgttgttacaagtgaaacgcttcgaggttacagctactattgattaaggagatcgcctatggtctcgccccggcgtcgtgcgtccgccgcgagccagatctcgcctacttcataaacgtcctcataggcacggaatggaatgatgacatcgatcgccgtagagagcatgtcaatcagtgtgcgatcttccaagctagcaccttgggcgctacttttgacaagggaaaacagtttcttgaatccttggattggattcgcgccgtgtattgttgaaatcgatcccggatgtcccgagacgacttcactcagataagcccatgctgcatcgtcgcgcatctcgccaagcaatatccggtccggccgcatacgcagacttgcttggagcaagtgctcggcgctcacagcacccagcccagcaccgttcttggagtagagtagtctaacatgattatcgtgtggaatgacgagttcgagcgtatcttctatggtgattagcctttcctggggggggatggcgctgatcaaggtcttgctcattgttgtcttgccgcttccggtagggccacatagcaacatcgtcagtcggctgacgacgcatgcgtgcagaaacgcttccaaatccccgttgtcaaaatgctgaaggatagcttcatcatcctgattttggcgtttccttcgtgtctgccactggttccacctcgaagcatcataacgggaggagacttctttaagaccagaaacacgcgagcttggccgtcgaatggtcaagctgacggtgcccgagggaacggtcggcggcagacagatttgtagtcgttcaccaccaggaagttcagtggcgcagagggggttacgtggtccgacatcctgctttctcagcgcgcccgctaaaatagcgatatcttcaagatcatcataagagacgggcaaaggcatcttggtaaaaatgccggcttggcgcacaaatgcctctccaggtcgattgatcgcaatttcttcagtcttcgggtcatcgagccattccaaaatcggcttcagaagaaagcgtagttgcggatccacttccatttacaatgtatcctatctctaagcggaaatttgaattcattaagagcggcggttcctcccccgcgtggcgccgccagtcaggcggagctggtaaacaccaaagaaatcgaggtcccgtgctacgaaaatggaaacggtgtcaccctgattcttcttcagggttggcggtatgttgatggttgccttaagggctgtctcagttgtctgctcaccgttattttgaaagctgttgaagctcatcccgccacccgagctgccggcgtaggtgctagctgcctggaaggcgccttgaacaacactcaagagcatagctccgctaaaacgctgccagaagtggctgtcgaccgagcccggcaatcctgagcgaccgagttcgtccgcgcttggcgatgttaacgagatcatcgcatggtcaggtgtctcggcgcgatcccacaacacaaaaacgcgcccatctccctgttgcaagccacgctgtatttcgccaacaacggtggtgccacgatcaagaagcacgatattgttcgttgttccacgaatatcctgaggcaagacacactttacatagcctgccaaatttgtgtcgattgcggtttgcaagatgcacggaattattgtcccttgcgttaccataaaatcggggtgcggcaagagcgtggcgctgctgggctgcagctcggtgggtttcatacgtatcgacaaatcgttctcgccggacacttcgccattcggcaaggagttgtcgtcacgcttgccttcttgtcttcggcccgtgtcgccctgaatggcgcgtttgctgaccccttgatcgccgctgctatatgcaaaaatcggtgtttcttccggccgtggctcatgccgctccggttcgcccctcggcggtagaggagcagcaggctgaacagcctcttgaaccgctggaggatccggcggcacctcaatcggagctggatgaaatggcttggtgtttgttgcgatcaaagttgacggcgatgcgttctcattcaccttcttttggcgcccacctagccaaatgaggcttaatgataacgcgagaacgacacctccgacgatcaatttctgagaccccgaaagacgccggcgatgtttgtcggagaccagggatccagatgcatcaacctcatgtgccgcttgctgactatcgttattcatcccttcgcccccttcaggacgcgtttcacatcgggcctcaccgtgcccgtttgcggcctttggccaacgggatcgtaagcggtgttccagatacatagtactgtgtggccatccctcagacgccaacctcgggaaaccgaagaaatctcgacatcgctccctttaactgaatagttggcaacagcttccttgccatcaggattgatggtgtagatggagggtatgcgtacattgcccggaaagtggaataccgtcgtaaatccattgtcgaagacttcgagtggcaacagcgaacgatcgccttgggcgacgtagtgccaattactgtccgccgcaccaagggctgtgacaggctgatccaataaattctcagctttccgttgatattgtgcttccgcgtgtagtctgtccacaacagccttctgttgtgcctcccttcgccgagccgccgcatcgtcggcggggtaggcgaattggacgctgtaatagagatcgggctgctctttatcgaggtgggacagagtcttggaacttatactgaaaacataacggcgcatcccggagtcgcttgcggttagcacgattactggctgaggcgtgaggacctggcttgccttgaaaaatagataatttccccgcggtagggctgctagatctttgctatttgaaacggcaaccgctgtcaccgtttcgttcgtggcgaatgttacgaccaaagtagctccaaccgccgtcgagaggcgcaccacttgatcgggattgtaagccaaataacgcatgcgcggatctagcttgcccgccattggagtgtcttcagcctccgcaccagtcgcagcggcaaataaacatgctaaaatgaaaagtgcttttctgatcatggttcgctgtggcctacgtttgaaacggtatcttccgatgtctgataggaggtgacaaccagacctgccgggttggttagtctcaatctgccgggcaagctggtcaccttttcgtagcgaactgtcgcggtccacgtactcaccacaggcattttgccgtcaacgacgagggtccttttatagcgaatttgctgcgtgcttggagttacatcatttgaagcgatgtgctcgacctccaccctgccgcgtttgccaagaatgacttgaggcgaactgggattgggatagttgaagaattgctggtaatcctggcgcactgttggggcactgaagttcgataccaggtcgtaggcgtactgagcggtgtcggcatcataactctcgcgcaggcgaacgtactcccacaatgaggcgttaacgacggcctcctcttgagttgcaggcaatcgcgagacagacacctcgctgtcaacggtgccgtccggccgtatccatagatatacgggcacaagcctgctcaacggcaccattgtggctatagcgaacgcttgagcaacatttcccaaaatcgcgatagctgcgacagctgcaatgagtttggagagacgtcgcgccgatttcgctcgcgcggtttgaaaggcttctacttccttatagtgctcggcaaggctttcgcgcgccactagcatggcatattcaggccccgtcatagcgtccacccgaattgccgagctgaagatctgacggagtaggctgccatcgccccacattcagcgggaagatcgggcctttgcagctcgctaatgtgtcgtttgtctggcagccgctcaaagcgacaactaggcacagcaggcaatacttcatagaattctccattgaggcgaatttttgcgcgacctagcctcgctcaacctgagcgaagcgacggtacaagctgctggcagattgggttgcgccgctccagtaactgcctccaatgttgccggcgatcgccggcaaagcgacaatgagcgcatcccctgtcagaaaaaacatatcgagttcgtaaagaccaatgatcttggccgcggtcgtaccggcgaaggtgattacaccaagcataagggtgagcgcagtcgcttcggttaggatgacgatcgttgccacgaggtttaagaggagaagcaagagaccgtaggtgataagttgcccgatccacttagctgcgatgtcccgcgtgcgatcaaaaatatatccgacgaggatcagaggcccgatcgcgagaagcactttcgtgagaattccaacggcgtcgtaaactccgaaggcagaccagagcgtgccgtaaaggacccactgtgccccttggaaagcaaggatgtcctggtcgttcatcggaccgatttcggatgcgattttctgaaaaacggcctgggtcacggcgaacattgtatccaactgtgccggaacagtctgcagaggcaagccggttacactaaactgctgaacaaagtttgggaccgtcttttcgaagatggaaaccacatagtcttggtagttagcctgcccaacaattagagcaacaacgatggtgaccgtgatcacccgagtgataccgctacgggtatcgacttcgccgcgtatgactaaaataccctgaacaataatccaaagagtgacacaggcgatcaatggcgcactcaccgcctcctggatagtctcaagcatcgagtccaagcctgtcgtgaaggctacatcgaagatcgtatgaatggccgtaaacggcgccggaatcgtgaaattcatcgattggacctgaacttgactggtttgtcgcataatgttggataaaatgagctcgcattcggcgaggatgcgggcggatgaacaaatcgcccagccttaggggagggcaccaaagatgacagcggtcttttgatgctccttgcgttgagcggccgcctcttccgcctcgtgaaggccggcctgcgcggtagtcatcgttaataggcttgtcgcctgtacattttgaatcattgcgtcatggatctgcttgagaagcaaaccattggtcacggttgcctgcatgatattgcgagatcgggaaagctgagcagacgtatcagcattcgccgtcaagcgtttgtccatcgtttccagattgtcagccgcaatgccagcgctgtttgcggaaccggtgatctgcgatcgcaacaggtccgcttcagcatcactacccacgactgcacgatctgtatcgctggtgatcgcacgtgccgtggtcgacattggcattcgcggcgaaaacatttcattgtctaggtccttcgtcgaaggatactgatttttctggttgagcgaagtcagtagtccagtaacgccgtaggccgacgtcaacatcgtaaccatcgctatagtctgagtgagattctccgcagtcgcgagcgcagtcgcgagcgtctcagcctccgttgccgggtcgctaacaacaaactgcgcccgcgcgggctgaatatatagaaagctgcaggtcaaaactgttgcaataagttgcgtcgtcttcatcgtttcctaccttatcaatcttctgcctcgtggtgacgggccatgaattcgctgagccagccagatgagttgccttcttgtgcctcgcgtagtcgagttgcaaagcgcaccgtgttggcacgccccgaaagcacggcgacatattcacgcatatcccgcagatcaaattcgcagatgacgcttccactttctcgtttaagaagaaacttacggctgccgaccgtcatgtcttcacggatcgcctgaaattccttttcggtacatttcagtccatcgacataagccgatcgatctgcggttggtgatggatagaaaatcttcgtcatacattgcgcaaccaagctggctcctagcggcgattccagaacatgctctggttgctgcgttgccagtattagcatcccgttgttttttcgaacggtcaggaggaatttgtcgacgacagtcgaaaatttagggtttaacaaataggcgcgaaactcatcgcagctcatcacaaaacggcggccgtcgatcatggctccaatccgatgcaggagatatgctgcagcgggagcgcatacttcctcgtattcgagaagatgcgtcatgtcgaagccggtaatcgacggatctaactttacttcgtcaacttcgccgtcaaatgcccagccaagcgcatggccccggcaccagcgttggagccgcgctcctgcgccttcggcgggcccatgcaacaaaaattcacgtaaccccgcgattgaacgcatttgtggatcaaacgagagctgacgatggataccacggaccagacggcggttctcttccggagaaatcccaccccgaccatcactctcgatgagagccacgatccattcgcgcagaaaatcgtgtgaggctgctgtgttttctaggccacgcaacggcgccaacccgctgggtgtgcctctgtgaagtgccaaatatgttcctcctgtggcgcgaaccagcaattcgccaccccggtccttgtcaaagaacacgaccgtacctgcacggtcgaccatgctctgttcgagcatggctagaacaaacatcatgagcgtcgtcttacccctcccgataggcccgaatattgccgtcatgccaacatcgtgctcatgcgggatatagtcgaaaggcgttccgccattggtacgaaatcgggcaatcgcgttgccccagtggcctgagctggcgccctctggaaagttttcgaaagagacaaaccctgcgaaattgcgtgaagtgattgcgccagggcgtgtgcgccacttaaaattccccggcaattgggaccaataggccgcttccataccaataccttcttggacaaccacggcacctgcatccgccattcgtgtccgagcccgcgcgcccctgtccccaagactattgagatcgtctgcatagacgcaaaggctcaaatgatgtgagcccataacgaattcgttgctcgcaagtgcgtcctcagcctcggataatttgccgatttgagtcacggctttatcgccggaactcagcatctggctcgatttgaggctaagtttcgcgtgcgcttgcgggcgagtcaggaacgaaaaactctgcgtgagaacaagtggaaaatcgagggatagcagcgcgttgagcatgcccggccgtgtttttgcagggtattcgcgaaacgaatagatggatccaacgtaactgtcttttggcgttctgatctcgagtcctcgcttgccgcaaatgactctgtcggtataaatcgaagcgccgagtgagccgctgacgaccggaaccggtgtgaaccgaccagtcatgatcaaccgtagcgcttcgccaatttcggtgaagagcacaccctgcttctcgcggatgccaagacgatgcaggccatacgctttaagagagccagcgacaacatgccaaagatcttccatgttcctgatctggcccgtgagatcgttttccctttttccgcttagcttggtgaacctcctctttaccttccctaaagccgcctgtgggtagacaatcaacgtaaggaagtgttcattgcggaggagttggccggagagcacgcgctgttcaaaagcttcgttcaggctagcggcgaaaacactacggaagtgtcgcggcgccgatgatggcacgtcggcatgacgtacgaggtgagcatatattgacacatgatcatcagcgatattgcgcaacagcgtgttgaacgcacgacaacgcgcattgcgcatttcagtttcctcaagctcgaatgcaacgccatcaattctcgcaatggtcatgatcgatccgtcttcaagaaggacgatatggtcgctgaggtggccaatataagggagatagatctcaccggatctttcggtcgttccactcgcgccgagcatcacaccattcctctccctcgtgggggaaccctaattggatttgggctaacagtagcgcccccccaaactgcactatcaatgcttcttcccgcggtccgcaaaaatagcaggacgacgctcgccgcattgtagtctcgctccacgatgagccgggctgcaaaccataacggcacgagaacgacttcgtagagcgggttctgaacgataacgatgacaaagccggcgaacatcatgaataaccctgccaatgtcagtggcaccccaagaaacaatgcgggccgtgtggctgcgaggtaaagggtcgattcttccaaacgatcagccatcaactaccgccagtgagcgtttggccgaggaagctcgccccaaacatgataacaatgccgccgacgacgccggcaaccagcccaagcgaagcccgcccgaacatccaggagatcccgatagcgacaatgccgagaacagcgagtgactggccgaacggaccaaggataaacgtgcatatattgttaaccattgtggcggggtcagtgccgccacccgcagattgcgctgcggcgggtccggatgaggaaatgctccatgcaattgcaccgcacaagcttggggcgcagctcgatatcacgcgcatcatcgcattcgagagcgagaggcgatttagatgtaaacggtatctctcaaagcatcgcatcaatgcgcacctccttagtataagtcgaataagacttgattgtcgtctgcggatttgccgttgtcctggtgtggcggtggcggagcgattaaaccgccagcgccatcctcctgcgagcggcgctgatatgacccccaaacatcccacgtctcttcggattttagcgcctcgtgatcgtcttttggaggctcgattaacgcgggcaccagcgattgagcagctgtttcaacttttcgcacgtagccgtttgcaaaaccgccgatgaaattaccggtgttgtaagcggagatcgcccgacgaagcgcaaattgcttctcgtcaatcgtttcgccgcctgcataacgacttttcagcatgtttgcagcggcagataatgatgtgcacgcctggagcgcaccgtcaggtgtcagaccgagcatagaaaaatttcgagagtttatttgcatgaggccaacatccagcgaatgccgtgcatcgagacggtgcctgacgacttgggttgcttggctgtgatcttgccagtgaagcgtttcgccggtcgtgttgtcatgaatcgctaaaggatcaaagcgactctccaccttagctatcgccgcaagcgtagatgtcgcaactgatggggcacacttgcgagcaacatggtcaaactcagcagatgagagtggcgtggcaaggctcgacgaacagaaggagaccatcaaggcaagagaaagcgaccccgatctcttaagcataccttatctccttagctcgcaactaacaccgcctctcccgttggaagaagtgcgttgttttatgttgaagattatcgggagggtcggttactcgaaaattttcaattgcttctttatgatttcaattgaagcgagaaacctcgcccggcgtcttggaacgcaacatggaccgagaaccgcgcatccatgactaagcaaccggatcgacctattcaggccgcagttggtcaggtcaggctcagaacgaaaatgctcggcgaggttacgctgtctgtaaacccattcgatgaacgggaagcttccttccgattgctcttggcaggaatattggcccatgcctgcttgcgctttgcaaatgctcttatcgcgttggtatcatatgccttgtccgccagcagaaacgcactctaagcgattatttgtaaaaatgtttcggtcatgcggcggtcatgggcttgacccgctgtcagcgcaagacggatcggtcaaccgtcggcatcgacaacagcgtgaatcttggtggtcaaaccgccacgggaacgtcccatacagccatcgtcttgatcccgctgtttcccgtcgccgcatgttggtggacgcggacacaggaactgtcaatcatgacgacattctatcgaaagccttggaaatcacactcagaatatgatcccagacgtctgcctcacgccatcgtacaaagcgattgtagcaggttgtacaggaaccgtatcgatcaggaacgtctgcccagggcgggcccgtccggaagcgccacaagatgacattgatcacccgcgtcaacgcgcggcacgcgacgcggcttatttgggaacaaaggactgaacaacagtccattcgaaatcggtgacatcaaagcggggacgggttatcagtggcctccaagtcaagcctcaatgaatcaaaatcagaccgatttgcaaacctgatttatgagtgtgcggcctaaatgatgaaatcgtccttctagatcgcctccgtggtgtagcaacacctcgcagtatcgccgtgctgaccttggccagggaattgactggcaagggtgctttcacatgaccgctcttttggccgcgatagatgatttcgttgctgctttgggcacgtagaaggagagaagtcatatcggagaaattcctcctggcgcgagagcctgctctatcgcgacggcatcccactgtcgggaacagaccggatcattcacgaggcgaaagtcgtcaacacatgcgttataggcatcttcccttgaaggatgatcttgttgctgccaatctggaggtgcggcagccgcaggcagatgcgatctcagcgcaacttgcggcaaaacatctcactcacctgaaaaccactagcgagtctcgcgatcagacgaaggccttttacttaacgacacaatatccgatgtctgcatcacaggcgtcgctatcccagtcaatactaaagcggtgcaggaactaaagattactgatgacttaggcgtgccacgaggcctgagacgacgcgcgtagacagttttttgaaatcattatcaaagtgatggcctccgctgaagcctatcacctctgcgccggtctgtcggagagatgggcaagcattattacggtcttcgcgcccgtacatgcattggacgattgcagggtcaatggatctgagatcatccagaggattgccgcccttaccttccgtttcgagttggagccagcccctaaatgagacgacatagtcgacttgatgtgacaatgccaagagagagatttgcttaacccgatttttttgctcaagcgtaagcctattgaagcttgccggcatgacgtccgcgccgaaagaatatcctacaagtaaaacattctgcacaccgaaatgcttggtgtagacatcgattatgtgaccaagatccttagcagtttcgcttggggaccgctccgaccagaaataccgaagtgaactgacgccaatgacaggaatcccttccgtctgcagataggtaccatcgatagatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccggtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccaagcactcccgaggggaaccctgtggttggcatgcacatacaaatggacgaacggataaaccttttcacgcccttttaaatatccgttattctaataaacgctcttttctcttag

SEQ ID NO: 136 - DNA, 1000 bp, Zea mays
  source 1..1000: mol_type = genomic DNA
tgccacgcaaactaaaaggcaaattctacaggacagcaatccggccggctatgttgtatggagcagaatgttggcccactaaaagacgacatgtccaacaactaagtgtggcagagatgcgtatgttgcgctggatatgtggccacacaaggagagatcgagtccggaatgatgatatacgagagagagtaggagtggcgccaattgaggagaagcttatgcaacatcgcttgagatggtttggacatatccaacgaagacctgaagaggcaccagtgcatatcggaataattaggcgtcccgaaaatgtgaagagaggtagaggtcgaccaactttgacgtggacagaggctgtgaagagagacctgaaggagtggaataatgacaaagagctcgccgcagataggaaggggtggaagtgtgcaattcacgtgccagaaccctgattgatagtttcgcttttcctccttaatcgtttgaccttttcttgtgtccattttagatcttgctggtccttgtgggttttatctcttttatgtgtttccccgtttcgttgttttcggttctcctttgcctttgtttcccttttctgttctttgggggttgagctctgaggttttcatacggggtttcatctctagcctaccccaacgtgcttgggacaaaaaggctttgttgttgttgttgttgttgtatctgtatcctaaaaggtgagagagaagggttattaagaaaaaccctcgtcgctggccactgaaggccgggcccaatttagaacctagacctgctgccaccgcactacaagaccgaggcctaaaaggcccatcaggaggcgcatcggcgaatgccccaaactaaaaccctaccccggcaagtatatatatcctcccaacctcagttcttgttcccattatcacggcggcggtggcggagcgtaaggcgaaggagtagcagcagcaggcggcgccgagtagcggctccccatctcgagcttgccacc

SEQ ID NO: 137 - DNA, 855 bp, Zea mays
  source 1..855: mol_type = genomic DNA
gtaaggttcccttccctcctcccctcacacccctgttcgtgttccttcggatcggatctcagtggtgatgttagacgtccgcggctgcctacgtagtggcattgccgcccgaaaggtttgtttaggtggggtagatccgaaacaggccggatctggaccatgtccgcggcggggcggcgggacttgatcgcgtagctgtcgtgtgcatttctccctaccagtggcggaatcggcgatgtggacctaagggctaaggcttatctgctgccttgaccatttcgtcgctgacaaaaacaaagtgacaatcatgccgttctctgtttgtttatctggatcgttattacgctgtgaatcctgcgatatgtggctaagtgatttttcttctttttctgggggcagtttagcctttgacccagtcctaggtgtggtcactaggactgtgtagcatgatgagtgaggttgcagcaggctgattgctagtggacgttttttccccaatttgttaggttttcacgctccaggttgtgcaagtaattttgctagtgattgtgtgatccatcttcaacgttgaaccttgtttttccccctaaaacccccaacaggaaatcttgccccgacttctattgcaaaaattgtaacgcttagcaccctgattgactcaattcctgtcactaggcatgctcggtcaaaagcagatgatttaccacttagaaactgccctgcccctgctttccacatagcatttcgaactttttgactactattgacacccccctaacttgccgaactatttctctcttcagctactatttacctagttataattacataaatgtttgtgtgtatcttgtgcag

SEQ ID NO: 138 - DNA, 884 bp, Zea mays
  source 1..884: mol_type = genomic DNA
gtgaggcatccgatcgatttttctttctttctttactacactccttcgcgatatggggacggcactcggtagtggcgtgaggtgcggtaaatcgcgttagtttagttgtagggtttgatcgcttcgggggggaccgggggttgggcttcccgtgttgaaccgtcaatcggacgtagtagtagtgcggattcggggtttgatcgacggaaagaggggttgtccgcactcttggtgtggttatagggttttgcgatttgtttgtctgtgtaggcccgtttcgtctcggggagtagattttcattgctactaccaatccctatgtgctttggtgaacacgtattttggtctgtatatggtttaaacgtgaagactatggtagtgtgagaccatgatttggatccttttctgtggcattatagttaaaatcgtgaggatctatcttttagcgcttagggtattgttatagacgagatcccctctttgggctctaaaaatagcaagaaaaggacatcttttgggcaagttaacgtcctgtattattctgaacgagatctgtttactttcttataagtttgatgttttggtctggaatatggttgcgttcatcgtccaattagtgtgtttgcagtatgtgttggtgtagttcctctgtgggcattttgtggccccagaaatgatagattttaagaaaggtttaggcagaaggggatcttaagtgttgtccagtacaaagtaacaatttgtagcacttgtttcttttcttttgtttgactatatgaaatttcggccatgtaattgtttcaaaataataagatcgaatagtgttgcacactacttcccagtcctatgtatacttatcagatttttcctctttgatatttcag

SEQ ID NO: 139 - DNA, 1487 bp, Zea mays
  source 1..1487: mol_type = genomic DNA
ccggctataccgctcccgccctcgtttttcagtaaaaaaaatatggtaatggaagtgggagagagtttttccaactgtttccgatcgttttcatccctatctataaacatccacatgagtaggggaggcggggtggcgagtggacgacactgtagccaacctaaggaccaaagctttagccttaaccattgcaccatgtgtcgcttattgttatatagagtatataaatgtatatagtaacaatttgaaaattaaaattaaaatcatgattgaataaaaatctcatttaaataaaaaattacatatatgatatatagaattcataacaatgtacgagtaactaactagttctatacttaagcataaatagaaagcgtagcaatgtatgcacactttgctagtcggatatttagatactagttagaagtattaaatatagtctaagtataaaactaattatatagatgaggactaaacagcaagacgaacctattaagtttaagtagtccatggttcgtccatgtaaaataaatatttgctaataatagattaattagacttaatagatccatctcgtcgtttagtctttatctatataattacttttgtagttagactatatttaattttagtaattgacatttaaacatccgatatgatccagacttgatgttagtcaggaaaaccaaacatccccttaaccatattggtcccaatttttggtgcctttacccatcaaatgatattcacacaatcacacatctgggcctaactttcatcgttgctgtccacgacggcgacctggaggcgaggtcaattccttggcccaagcatagcttggagcttgcacgctaagaagaggctctcgtactctacaaacagtacagcacatacaggtgacaaaacgacacacatcaaccagccaaataataaatgagcttcttcatgggcacggcaagccgacaactaccaacaagatacaggtgacaaaaagaaaacaagaggcccccactcaccagtgggtcgtaggcaacgcacgcggacgcggtccagcgggcgagaagatccccgacttgcgcccaaagaagatacaggatcaaggatttttaaccgcagttttctattccacgaccttatccacaccagcagattcgaaattcacggacaggcccatggacccggcgaaagccagcggtggttcagcccctgacgtgcgggtcccactctccagccgcaccgcctagagaggcagaggcatcccttcgcgtggaagcaaacgaggcgtgataaagtggggctcctcggtcccggcgttggccgcatcgacactcgccgcgcaccaccaccaccgctgcggctcacggctacgcagcccgctctcccgacccccccgtgccctcctctttttgctactagcacatagagtttcgcccgaatcgatcgccgactgactccgctagggttcggcccgatcgccgcttcgtcct

SEQ ID NO: 140 - DNA, 629 bp, Zea mays
  source 1..629: mol_type = genomic DNA
atcctctgaatgtgctgtgttgagagttttttctagttgctctgcaaggatatagaacaatgttctaagactaccatgtttttaagtctgcctgatgcttataattcatgaacgatttttgcagctagctgtaatgtgactatttatcttatctgcttgttcaaccctgttgtcgtgtgttgattctctgtttcatggtcttttcactcgacagaaacagttatttcttaagaacttcaccattatattcacagctgtgaactatgatattggaagtctttggtcatttttcgttgaactattttgctgaaagttttttccgagaaagatgccagaccggtctccgatctaagaatggccataactgatgagcatcacaaggtatatattagttgatcaaatgtctttgagtacatctgtttgacacagcttatttttagcttcttcacatatttaagcatagcttatttttagcttcttttaatatttaagcagaagttttactaaactggtagcatctccagcagcttattagttcagctgactcttagaatgaactaaaagcagccaacaagtataagctattttttaccagtttcttagataacttgtttttctaacaaatagtg

SEQ ID NO: 141 - DNA, 607 bp, Zea mays
  source 1..607: mol_type = genomic DNA
gccatcagtcgttgaagctgctgctgtatctgggttatctagtgtctctgccattgcccatggatggtgctgtctttcaaagtatttgtatggtttgtgtcgtgagtcgtgactgagctggtttcatggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttcattttcatttgaagtttgattagagctattgataccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgca

SEQ ID NO: 142 - DNA, 646 bp, Zea mays
  source 1..646: mol_type = genomic DNA
cgcttttagtatggcctcggaaacatggcctgcctgttgctagtagaaagctagcactgcccttttggtaagggcgtttcaggagcactgtgctggactatatagagagatgctcctctgtgactgtgaccacctcacgttctgcgtactctgtaatctggtaggatgcctgtctctatcatgattcatgaaggacgattggtctgtcttttttttatcgtgctatttattaatcgtgtaaatgtactagcgaagggaaggcactggtagctaggttagctcgagtccggatggaatgataatgctactaatacaaacaatgccgtagtgtgtgtgtgtatttatcaactcgcgtgtcatcacgtccgttaagttgccgctggtatctccctccctgtcggtggcgcgtattctcggcgcactcctctgaaccagcatgaattcagagcaaggtagaggggcctgtaaaccgtccaccatctgtctagctgttcttggtagaacacttgcagagattctcagctctctctctctctctctctctctctctctctctctcgtcccgtcctagcggacaagcggagtggggctccctcgctctgtgtcactgcttgctgcatcgctgaaagcggtagatgcggcggta

SEQ ID NO: 143 - DNA, 936 bp, Zea mays
  source 1..936: mol_type = genomic DNA
agatcgtgtgctatctaagtatatattcgtaaataatgagacggctgtgccactgcggtgcagccctggtgcgtctcagagcttgcttatggtgaactcttcgtgttaggtttcttccttatatctgtcgtgtctgcggtgtcgatgaactatctagtatgtggagcgtgtgcgtctcagcagttaccctgccatgccacgtggactggctgtcgtgcctactgtctgttaccatgtatatatataaaaaatgatgcgtggaccaatgtttgatgctggcgctcttgccctcgtgataatgtattagcggttggcttgttgcttgcttgtcagccccaggtaaatcttgctgcttgctttgcttgcagaagcgagcgtctgcacgtacgtaatcaggataactgtatgcagcgatttggtgatatgcactgccagtggagtttgcttcacaaagaatggaatggaacctggcgaccctacgtggagtttgcgcaccgatgtaaggagctggaggccggctccgaggactattgcttccacttcctatatgtggcacgctgaagggcaatgccgtgttttagttgaaattttggccagtccacgagcaatgtggtgtttctactctctgtatctctactaatattaccaaaatcaaatcctctgattatctctacctgtctttatcagccattggtattttgggtgtgtgtgtgtttcatctttatctacgtgcttctgtagaaagatggcaggcaaaatcgtttgggttgtagttgtaggtagtagctacaaagagacgagacttgggaggtgattatcttttgtagagtgaatagcttgaatgaataaccgtttgtgtctatgtaggacccactgaatattcaggagaaaaactgttgtcatatattgcgtgctattcaagctcgcttctgcctt

SEQ ID NO: 144 - DNA, 16437 bp, synthetic construct
  misc_feature 1..16437: note = Vector sequence
  source 1..16437: mol_type = other DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttaccagagctggtcacctttgtccaccaagatggaactgcggcctcgaagctggcgcgccgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaaggtaccgatccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagggccggccatcaacaactctcctggcgcaccatcgtcggctacagcctcggtgacgtggggcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgcgatctgagcttctagaggatccccatcgatgggccccggccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgggcccaggtagaatccgcctgagtcgcaagggtgacttcgcctatattggacgacggcgcgcagagggcgacctctttttgggttacgattgtaggattatcactaaaacaatacatgaacatattcaaatggcaatctctctaaggcattggaaataaatacaaataacagttgggtggagtttttcgacctgagggcgttaaccttctgttaacctaaaagctcttgcccaaacagcagaatcggcgctaattgccagcggcggaacttttccagtttcgcgaaaaatatcgccactggcaaggaatgggtttgagatggcgaagtctgtcctaaaagcagcgcctgtagttgtagggttgacggccttgatggagcgtcatgccgatgccctctcgagccaacttcaagcacatcatcttaaggttttcccgccgcattccgagaagggcattcgaacattcgggccatcggaggcgtccaagctgctcggcgttggcgagtcatatttacggcagaccgcgtctgagatgccagagttgaatgttagcatgagcccgggtggcaggcgaatgttctcaattgaagatatccatgtgattcggaagtatatggatcaggtcggccgcgggaaccggcgctacctgccacatcgtcgaggcggcgagcagcttcaggttatctctgtgatgaatttcaaaggtgggtcgggtaagaccaccaccgccgcgcatctggcgcagtacctcgctatgcgcggatatcgagtcttggccattgatctcgatcctcaagcgagcctttctgcactctttgggagccaaccggagacggacgttggcccgaacgaaacgctctacggcgctataaggtatgatgatgagcaggtggcaatcgaacgagtcgtccgagggacttacattcccgacctccacctgattcctggtaaccttgagctgatggagtttgaacacgatacgccacgcgcgctgatgaaccgcaaagagggcgacacgctcttttatggtcgcatcagccaagtaattgaagatatcgcggataactatgacgtcgtggtcatcgactgccctccccagcttgggtatctcacgctatccgcattgactgcggcgacgtccattcttgtcacggtccatccgcagatgctggatgtgatgtcgatgaaccagtttctggcaatgacatcgaaccttttgcgtgaaatcgagaatgctggcgccaagttcaagtttaattggatgcgctatctgataacccgtttcgaaccgagcgacggaccacagaaccaaatggtaggttatctgcggtcgatttttggcgaaaatgtcctcaattttccgatgcttaaaaccaccgcggtttcggacgctggcctgacaaaccagactctattcgaagtggagcgtggcctgttcacgcgctcgacctatgatcgagccttggaggcgatgaacgccgtcaacgacgagatcgaaacactgatcaaaaaagcatggggtaggcccacatgagccggaagcacatccttggcgtctcaactgacgcccctgagacgtcgcccgccgacaataggacggcaaagaaccgctccatgccgctcctcggcgtaacaaggaaggagcgcgatccggcaacgaagctcacagcgaacattggtaacgcactgcgagagcaaaacgatcgtcttagccgtgccgaagagatcgagcggcgtctcgctgaaggtcaggcagtgatagagttggatgcctcgtcaatagaaccgtctttcgtgcaggatcgtatgcgaggggacattgacgggctccttacttcgatccgggaacaaggacagcaagtcccaatccttgtgcgaccgcatccgagccagccgggccgatatcaggttgccttcggccaccgccggctacgcgccgtttcagaactcggacttccggtcagagcggtcgttcgcgaactgacggacgagcaagtggtcgtagcacagggtcaggaaaacaatgagcgcgaagatcttaccttcatcgaaaaggcgcgcttcgcacatcgcctgaacaggcagttttctcgagagattgtcatcgccgcgatgtcgatcgacaagagcaatttgtccaagatgcttctgctcgttgacgccctcccctctgaactgaccgatgctattggtgccgctcctggtgttggacggccgagttggcaacaacttgccgagctgattgagaaagtttcttcaccggccgacgtggctaaatatgctatgtcggaggaagttcaagcgctgccatcggcagaacgattcaaggcggtgatcgctagtctgaagcccagtcgggttgcgcgtggacttcccgaggtcatggccaccccagacggcaccagaattgcacaggtgacgcagagcaaggccaaactggaaatcacgattgacaggaaggcgacgcccgattttgcgaccttcgtgctcgatcatgtgccagcgctgtatcaagcgtaccacgctgagaaccaacggaaacggggagagtaaaccgcaaaagaaaagagccccctcaacgtcgccgtcgcggaagcccttctgtctctctagcgcgaacagaatcgcatttcctcgaatcctcgtcaagagtttttagcgccgttttggtgagctgatttcctttgcctgctgaaaggtgaaagatgatgcagacaggaagtgtaacgacgccattcgggcggcggccaatgacgcttgcgcttgtgcggcgccagacggcgctggccgatatcaaacaaggcaagacagcggacaagtggaaggtctttagagacgcgtccgcggctatggaactacttggaatccagtccaacagtcttgccgtccttgatgcgctattgagctttcacccggaaacggagttgcgtcaggaggcacagctgatcgtcttcccgtcgaatgctcagcttgcccttcgggcgcatgggatggctggcgcgactttgcgtaggcacatcgccatgctcgtggagtcaggcttgatcgtccggaaggatagcgccaacggaaagcgttacgctcgtaaggatggcgctggtcagatcgagcgcgcgtttggcttcgatttgtctccgcttctcgcgcggtccgaagagctagcgatgatggcacagcaggtgatggccgatcgagcagcattcaggatggccaaagaaagtctgacgatttgccgacgggacgttcggaagctaattacggcagctatggaagagggagcggagggcgactggcaagctgtcgaggaagtctatgtggaacttgtgggtagaattccacgcgccccgacgcttgctgatgtagagtcaattctcgaagagatgtggatgctccaggaagagataatcaaccggttggaaattagagacaattcagaaaataatagcaccaatgctgcccagagcgagcagcacatacagaattcaaaacccgaatccgttaatgaacttgaacctcgctctgaaaaggagcagggcgctaagccgagtgaaatagaccgggcaaggagcgagccgataaaagcgttccccctcgggatgatcctgaaagcatgcccgaccattggcaattatgggccgagcggtgcggttgctagctggcgtgacctcatgtcggctgcggtggtggttcggtctatgctgggggtcagcccgtcggcttaccaagacgcgtgtgaggcaatgggaccggagaatgcggcagcagcgatggcgtgcattttggagcgagcgaacttcatcaattcgcccgggggctatctccgagatctgacacggcggagcgagcttgggaagttttcacttggcccgatgataatggcgctcttgaaggctagcgggcaggggacgttgcggtttggctagaattagcgagtatggagcaggatggtctgtggtcagctgaccacagacctaataggttgaaaacatgagcgttttttggatgatcgacagaccatccgattcccggagtaccaagcgtgctctgatgggagcgataacattactcaacaagcacgaaggccccatgccgatcgttgatcgtgaaggagagcctgctctacatgcggcggtattttgccggccgaggcatgtagtcgcggagcactgcctatttactgccctaggcacaaacgttgactcttggatcgagctggcagacaaagcaataacccacacagaggacgattaatggctgacgaagagatccagaatccgccggacggtactgctgctgccgaagttgagccggctgctcctagaggtagaagagcaaagaaagcaccagccgaaacagcccgcacgggatcgttcaaatccgtgaagccgaaaacccgcggcctcagcaaccgagaaaaactggagaagatcggtcaaatcgaagctcaggtcgctggcggcgcaaccttgaaggacgccgttaagatcgtgggtatttccgttcagacctattatcaatggaagagagctgcggttcaacctgtctcacagaatccggccgtgtctgtttcagttgacgatgaactcggcgagttcatccaactcgaggaggaaaatatgcatggcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactatacgtaaccaactagtgcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagcgtaccgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttag

SEQ ID NO: 145 - DNA, 17 bp, synthetic construct
  misc_feature 1..17: note = MPSS promiscuous tag
  source 1..17: mol_type = other DNA
gatcaaaaaaaaaaaaa

SEQ ID NO: 146 - skipped